SEQUENCE LISTING (44 sequences)

SEQ ID NO: 1 - DNA, 153 bp, synthetic construct
  source 1..153: mol_type = other DNA
gggctggtgagatggctcagtgggtaagagcacccgactgctcttccgaaggtcaggagttcaaatcccagcaaccacatggtggctcacaaccatccgtaacgagatctgattccctcttctggagtgtctgaagacagctacagtgtactt

SEQ ID NO: 2 - DNA, 1002 bp, synthetic construct
  source 1..1002: mol_type = other DNA
ggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaag

SEQ ID NO: 3 - DNA, 1074 bp, synthetic construct
  source 1..1074: mol_type = other DNA
gagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatc

SEQ ID NO: 4 - DNA, 656 bp, synthetic construct
  source 1..656: mol_type = other DNA
gtcttttcgaggtaggagtcgactcctgtgaggtatggtgctgggtgcagatgcagtgtggctctggatagcaccttatggacagttgtgtccccaaggaaggatgagaatagctactgaagtcctaaagagcaagcctaactcaagccattggcacacaggcattagacagaaagctggaagttgaaatggtggagtccaacttgcctggaccagcttaatggttctgctcctggtaacgtttttatccatggatgacttgcttgggtaaggacatgaagacagttcctgtcataccttttaaaggtatggagagtcggcttgactacactgtgtggagcaagttttaaagaagcaaaggactcagaattcatgattgaagaaatgcaggcagacctgttatcctaaactagggtttttaatgaccacaacaagcaagcatgcagcttactgcttgaaagggtcttgcctcacccaagctagagtgcagtggcctttgaagcttactacagcctcaaacttctgggctcaagtgatcctcagcctcccagtggtctttgtagactgcctgatggagtctcatggcacaagaagattaaaacagtgtctccaattttaataaatttttgcaatccatcaaaaaaaaaaaaaaaaaa

SEQ ID NO: 5 - DNA, 1067 bp, synthetic construct
  source 1..1067: mol_type = other DNA
ggttgtatacttactgattaataataagagctctttacatattaaggaaattaacccttttcaaatacattcctatttctcactaatctttaagttttattgtaatattttgctctttagtttatatatatatgtatatatatatatatgtatatatatatatatacatatatatatacatatatatatactaattttcttttatggttcctggattttgtgagtagtttgaaaaggctaatccagctgaagattttgttgttgttgttaaaccccatgttttctcctaactctttttatttttattttggaggactctatctagacttaattttagcataacaagtgacagggttagttagcctgttgtccttacaccattttctggctaatacagctattaactattgatctgtctattcacgtgccagttcctaatggttttacatagtgtaatctgcacttcaaaatagcgaagggaagccctacctcattattctacttttccagaattctcctggctattccaggctgcatgtttaccttaaccttccctgtgatgtcttcatgccgttgtcttcttatgcaagaataaggtacgtctttccatccactcacgtctatttaatttgactttgcattacacagaaagctggtcttggtctgtctacctcggcatctagttgtcctcactgccccctagccgaccccaccccatctgactgactaccccatcacagagtacttttatttacgttttgctctgcctaatggttacttgatactgtcacgccgacagtgtccagttcagtggtctttgcagttgaaatgctcccgtacacactgtcttgttaaaaatgccagtaagttcatacaaacccagcttgcacccaaggtcacattcagagagcgtagggctgggatgggttgttttccaagcttctgccactgtgtggctagctcttcccactgggaagttctgtgtacccggaatgtcggagtggagtcctgttctagtgtccagcacctgaccctgtgcccaacccctcaacagcctattcctgctg

SEQ ID NO: 6 - DNA, 1067 bp, synthetic construct
  source 1..1067: mol_type = other DNA
ccacagcctgctggaactttttacaaaatatgttgccatgctggaccctgggcactggacataagccccctggcagcctttttcatgtcacccaaaggggtaattgtcctactggtggtctgtaagatgagttagggtgacttgctaatagacattgtaaatcttaatatttatgtatgtattttattattaccggttttccatttatgatggtaatattgtttcttctaagaatatttatttttccttctaaatattgagataaaattcatgcttttgaaatgttctattcagtggcttttagtatatttgctatgttgtgcaaccatcgacactatccatttctagaactttttcgtcatcccaaacagacgctctgtattcataaaaaaataacttcctacctgtctctccccctagtctttggtaacctttgttatactggtaaactttgttgtgctctctgtctgtgtgaatttgcctattctaggggcctcatataagtgtaatcatacagtatttgtctttttgggtctgtctgatttcacttagcgggttttcagggttcattcatgttgcagcatataacagtactgcgttcctttttctggctgaataatattccactgtatggatagaccccattttgtttattcacacatcatttggacatttggattatttctggtttttggctattatgaacaatggtgctatgaacagttgcgtacaagtttttgtgtgaacatatgttttcaattctctcattatatacctaggagtagaattactgggtcatatggtaactgtatatttttgaggaactgccaaactattttcccacgtccatgcaccatttcacattcccaccagtaagtaagagggttccaatttctgcgcattcttgccaacactagttattatctgactttctggttataatcattctaatgagtgtgaagtagcctctggtgtcatttggatttgcatttctctgatgagtgatgctatcaagcacctttgctggtgctgttggccatatgtgtatgttccctggagaagtgtct

SEQ ID NO: 7 - DNA, 797 bp, synthetic construct
  source 1..797: mol_type = other DNA
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatag

SEQ ID NO: 8 - DNA, 797 bp, synthetic construct
  source 1..797: mol_type = other DNA
gaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaa

SEQ ID NO: 9 - DNA, 1048 bp, synthetic construct
  source 1..1048: mol_type = other DNA
ttggccccactgtgcaccacacactcctttcccagcccaggggcacgcgaacaaaatgaggctcaagctgaccaggccgagccggaggaacgctggggcttggcagcagaagggatgggaccagagagaagggtgtggaggagaccccagtgagggccaggacatttcaggtaaagagaggtcatatctccgtacctcacttcctgacacaaacaagttttcactgttgtcagcaacaaagccctaatatagctgcggaagagaaaaactgcattgcattttgcctcctgcaagcatcatcaacagttactggaggaacgtaattccagaaagcttgaaagccgtggtgatggtaattatgtatcaaatgcctggttctatttctgttattattgttttgtcatttctgttttcccagcgatctgactgaactcgcagagggacaaatccagtttttctttttgacttttgtcaaactaaatcaggcctgatagaaaactcattgctctccggggaaacaaagtaggagccacgaaatgtcattttaacagagcgtgggtttggtgactgtaggaaaggatttgaggacgctccttctgttcggcttcctatgtcatgagcacaggctccacgcacgcacagacaccacggctcccggatgctgtggctccccgatcggggctcctgcagcgccagaagcccctccgggatgcttcgaggggctcccggtgggtggaggtacggacgccgctgcggccgccgccgccagtcctgctgctgttgttgctgctgcagtcacgtgggagcccctttaagtttccatagagaggcctctctggtgtcacatgatggacatgatataatgaaacaacattgtggagaggaaagcattaggggagcccacggctacaaaaacaagtgagtgagaagaggtgggaggaagagaaactacgccacctcccctgcagccgagtgcacgcagcagcctggcgtgacaagtgggcgacgccggggggcagggagccggggtccttggccctggccgggg

SEQ ID NO: 10 - DNA, 1048 bp, synthetic construct
  source 1..1048: mol_type = other DNA
aaatgaaggagttgagaagtacatactctgaggtttatcctaaataaactgaagtctaggcaagtggctgagctaggtttgccatgaatcaatctgctgagtatatttcttggtaactagttcatctttccttaattcagtcaacaaatatgatatcaagtttagtaagatgatgacagttgtcatgtaatactccatctggaagattgtatgcttctcagcttccctaacttttcttaatctaccttaagcttcctttctgaaaggtttctgtctttcctaacagtctcttctttccgccagctacctcttgtgtttaagaattaaaggaagatgagctaggaatcaagaaatacgcagaactgattcctgtcactggcgccaccctgccacttgcccagagctgtggtataaatttttttaataggctgaaataaaaatatgaatagcattttggtattaagcattaaattgataaaggctatgagatacacctgctctcaaccatgttaattttttattattggtattaataaattattacttactaacatattaacaagaattgcattgagaaacaaagcatccacaggccaaatcttctgaatttcaaatgtttatattaatgcgttgtattctagaaaagtagaattgtcttaagtagctttgtaatataaagtcatctatcagcccattacacctattagaatgttttaacctttatttcccacttttttgtttctagactgagtgtacttggtctggtatttgtggcataacttacggaacacataagaatgatacagatactatttaatgatgacctaatacaagcttagatagctaaggtgaaagcttctatggccttaacattttcctcttgaagaatgtattttctgtaataaaatacagtggctacttgaaatctataaacttatgtgaggtctggataaatctgagcaactttcttctttgtgctccaggaacctacgcactatatatataaataaagcttaagtaaacatcactgcaaaaaaaaaaaaaaaaa

SEQ ID NO: 11 - DNA, 673 bp, synthetic construct
  source 1..673: mol_type = other DNA
agagtgaaggagcaaaggggactcggccgccatgttaggagtactgggacgattccgcggagccgggcagaggttttaggggaatgattaacaaaggcgtccgaagaaatcgttgttggaaggtgaccaaggtggaaagagacgttgctttggccctgcaagtaagaagagagagggaatagcctgaaggagtaacactaaatttaaaatgacacttttttaccaaccagcgaaagcagatgttcaaaggggatattggccagagtctgtcacactaagatgagaaatgtcctttcttcctgaaggtgtctgatgtgtaaaaatatgatatactttgtgctgtttcctcccttcccttttgcatattattctgaaacaacattaactagttactttgcgtcattgaaggtatgcacttcccctctatgttaggagtgaataaaattaaaaatagatccttataacaaagaaaggcagatagaatgattaaaaatgaccaaaacatgttagaaacagtctctcaggtgtatgcagatggtaattacaaaaatactttttcaaaaaatgatcttctgtgtcatgtttctgggaacaagtcaagatgaatgagtttgatttttaagcagaagtagtatgtgttggtgtcatccatgaatacaacaatgaaaaaggc

SEQ ID NO: 12 - DNA, 653 bp, synthetic construct
  source 1..653: mol_type = other DNA
acaatccaattcctagatgctttctccagttatggccctgactgggatctccgaagaatgactcagctgaaactatttcatcattgcctacatttgtcttcagacgacctgcagtacaaacttggcttaaacctgcagttgacaatggattcaccgtgggagaataaattacaatggataaaaaacagcattacaatccctcaataaaatgggattttaacagacaaagctcctggtatgtggtcgttctcaggcccaaaggcccattttgcttttggctgcaactctgtaactctgtaacagagaccaagtgcctgcagggagcagatgtttggggcagtatctgctaaggtggctttgctgaaacttgtttctctcattgggtcttggaagtggcgaataggacgttcctatagaaaaactcagaaagagactgtgaaagataatcttaaagaggcactcacgaaacacaagctccttctccgggaaaaagaatcatcacccttgttatcccatgaaaattcatcttctccttttcagctgatatgaggacttgatctgtcctttcctataagtgccatctgtgtttaaatgaggctgttactctaactcaaagcagacttgcaagagtaaaataatcaatcttcatcata

SEQ ID NO: 13 - DNA, 75 bp, synthetic construct
  source 1..75: mol_type = other DNA
aaaaagcggtcaggcagctaaaccaaaaggtttagcaattgcctctgatgagtcgctgaaatgcgacgaaaaccg

SEQ ID NO: 14 - DNA, 86 bp, synthetic construct
  source 1..86: mol_type = other DNA
tgttttgtgttttggctgggtttttccttgttcgcaccggacacctccagtgaccagacggcaaggtttttatcccagtgtatatt

SEQ ID NO: 15 - DNA, 739 bp, synthetic construct
  source 1..739: mol_type = other DNA
cccttctccagagcgccgtcgaccccacgccacgcagaggtgaagatacgcaggacgtcctctaaagcaacttccgtgggtgccaggttgtgggctgggggcggtgctgaccttgagagagcggaggaggcgcaggcgcagcacagtttaaacttgattttgtgaggggacgaacagcgtgacaattaaaagaagatctgggaatcatccaatgtgataatttatgtgaaatctttctgcaaactatacagtatgataaaaatataaggtagtttcactggaaacaacagaagactagaagctgatgtgacgtggcaatatcaaccaggaatacccatctcgagttctggatatgtgctctgagaaggtgcccatgtcaccgaccacactggaggccaatgcagatactgggggaaggttccatggtagctaagtgtggacaagctaatcactgaagttccctgatgcagagttgataatctctctagagttggatgaaagactgtgctgccttgaagctctgagagatgccatgccaattcaaggcagggtctttattgttgaaactcttgcgtaatgacatctttattcaggtgaaaatacaggatgaatttcaactatatgatattgtttatgttcctcagacatgttatttgtctttacaaagattggtttcaataaactgtgtgactgatattaaataaacatggaattttacacattcataataaaaaaaaaa

SEQ ID NO: 16 - DNA, 51 bp, synthetic construct
  source 1..51: mol_type = other DNA
tctgcctccccagttcaagtggttgtcctgcctcagcctcccgagtagctg

SEQ ID NO: 17 - DNA, 51 bp, synthetic construct
  source 1..51: mol_type = other DNA
cccacctcccgggttcaagtgatcctcctgcctcagcctcccgagtagctg

SEQ ID NO: 18 - DNA, 44 bp, synthetic construct
  source 1..44: mol_type = other DNA
tccgcctcccgggttcaagtgattctcctacctcagcctcctca

SEQ ID NO: 19 - DNA, 51 bp, synthetic construct
  source 1..51: mol_type = other DNA
tccgcctcccaggttcaagcgattctcctgcctcaacctcctgagtagctg

SEQ ID NO: 20 - DNA, 41 bp, synthetic construct
  source 1..41: mol_type = other DNA
cgcctcccgggttcaagcgattctcctgctcagcctcccga

SEQ ID NO: 21 - DNA, 5452 bp, synthetic construct
  source 1..5452: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttagtcttttcgaggtaggagtcgactcctgtgaggtatggtgctgggtgcagatgcagtgtggctctggatagcaccttatggacagttgtgtccccaaggaaggatgagaatagctactgaagtcctaaagagcaagcctaactcaagccattggcacacaggcattagacagaaagctggaagttgaaatggtggagtccaacttgcctggaccagcttaatggttctgctcctggtaacgtttttatccatggatgacttgcttgggtaaggacatgaagacagttcctgtcataccttttaaaggtatggagagtcggcttgactacactgtgtggagcaagttttaaagaagcaaaggactcagaattcatgattgaagaaatgcaggcagacctgttatcctaaactagggtttttaatgaccacaacaagcaagcatgcagcttactgcttgaaagggtcttgcctcacccaagctagagtgcagtggcctttgaagcttactacagcctcaaacttctgggctcaagtgatcctcagcctcccagtggtctttgtagactgcctgatggagtctcatggcacaagaagattaaaacagtgtctccaattttaataaatttttgcaatccatcaaaaaaaaaaaaaaaaaa

SEQ ID NO: 22 - DNA, 5863 bp, synthetic construct
  source 1..5863: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattgg

SEQ ID NO: 23 - DNA, 5863 bp, synthetic construct
  source 1..5863: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaggttgtatacttactgattaataataagagctctttacatattaaggaaattaacccttttcaaatacattcctatttctcactaatctttaagttttattgtaatattttgctctttagtttatatatatatgtatatatatatatatgtatatatatatatatacatatatatatacatatatatatactaattttcttttatggttcctggattttgtgagtagtttgaaaaggctaatccagctgaagattttgttgttgttgttaaaccccatgttttctcctaactctttttatttttattttggaggactctatctagacttaattttagcataacaagtgacagggttagttagcctgttgtccttacaccattttctggctaatacagctattaactattgatctgtctattcacgtgccagttcctaatggttttacatagtgtaatctgcacttcaaaatagcgaagggaagccctacctcattattctacttttccagaattctcctggctattccaggctgcatgtttaccttaaccttccctgtgatgtcttcatgccgttgtcttcttatgcaagaataaggtacgtctttccatccactcacgtctatttaatttgactttgcattacacagaaagctggtcttggtctgtctacctcggcatctagttgtcctcactgccccctagccgaccccaccccatctgactgactaccccatcacagagtacttttatttacgttttgctctgcctaatggttacttgatactgtcacgccgacagtgtccagttcagtggtctttgcagttgaaatgctcccgtacacactgtcttgttaaaaatgccagtaagttcatacaaacccagcttgcacccaaggtcacattcagagagcgtagggctgggatgggttgttttccaagcttctgccactgtgtggctagctcttcccactgggaagttctgtgtacccggaatgtcggagtggagtcctgttctagtgtccagcacctgaccctgtgcccaacccctcaacagcctattcctgctg

SEQ ID NO: 24 - DNA, 5863 bp, synthetic construct
  source 1..5863: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccacagcctgctggaactttttacaaaatatgttgccatgctggaccctgggcactggacataagccccctggcagcctttttcatgtcacccaaaggggtaattgtcctactggtggtctgtaagatgagttagggtgacttgctaatagacattgtaaatcttaatatttatgtatgtattttattattaccggttttccatttatgatggtaatattgtttcttctaagaatatttatttttccttctaaatattgagataaaattcatgcttttgaaatgttctattcagtggcttttagtatatttgctatgttgtgcaaccatcgacactatccatttctagaactttttcgtcatcccaaacagacgctctgtattcataaaaaaataacttcctacctgtctctccccctagtctttggtaacctttgttatactggtaaactttgttgtgctctctgtctgtgtgaatttgcctattctaggggcctcatataagtgtaatcatacagtatttgtctttttgggtctgtctgatttcacttagcgggttttcagggttcattcatgttgcagcatataacagtactgcgttcctttttctggctgaataatattccactgtatggatagaccccattttgtttattcacacatcatttggacatttggattatttctggtttttggctattatgaacaatggtgctatgaacagttgcgtacaagtttttgtgtgaacatatgttttcaattctctcattatatacctaggagtagaattactgggtcatatggtaactgtatatttttgaggaactgccaaactattttcccacgtccatgcaccatttcacattcccaccagtaagtaagagggttccaatttctgcgcattcttgccaacactagttattatctgactttctggttataatcattctaatgagtgtgaagtagcctctggtgtcatttggatttgcatttctctgatgagtgatgctatcaagcacctttgctggtgctgttggccatatgtgtatgttccctggagaagtgtct

SEQ ID NO: 25 - DNA, 5593 bp, synthetic construct
  source 1..5593: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaagcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatag

SEQ ID NO: 26 - DNA, 5593 bp, synthetic construct
  source 1..5593: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaa

SEQ ID NO: 27 - DNA, 5844 bp, synthetic construct
  source 1..5844: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttattggccccactgtgcaccacacactcctttcccagcccaggggcacgcgaacaaaatgaggctcaagctgaccaggccgagccggaggaacgctggggcttggcagcagaagggatgggaccagagagaagggtgtggaggagaccccagtgagggccaggacatttcaggtaaagagaggtcatatctccgtacctcacttcctgacacaaacaagttttcactgttgtcagcaacaaagccctaatatagctgcggaagagaaaaactgcattgcattttgcctcctgcaagcatcatcaacagttactggaggaacgtaattccagaaagcttgaaagccgtggtgatggtaattatgtatcaaatgcctggttctatttctgttattattgttttgtcatttctgttttcccagcgatctgactgaactcgcagagggacaaatccagtttttctttttgacttttgtcaaactaaatcaggcctgatagaaaactcattgctctccggggaaacaaagtaggagccacgaaatgtcattttaacagagcgtgggtttggtgactgtaggaaaggatttgaggacgctccttctgttcggcttcctatgtcatgagcacaggctccacgcacgcacagacaccacggctcccggatgctgtggctccccgatcggggctcctgcagcgccagaagcccctccgggatgcttcgaggggctcccggtgggtggaggtacggacgccgctgcggccgccgccgccagtcctgctgctgttgttgctgctgcagtcacgtgggagcccctttaagtttccatagagaggcctctctggtgtcacatgatggacatgatataatgaaacaacattgtggagaggaaagcattaggggagcccacggctacaaaaacaagtgagtgagaagaggtgggaggaagagaaactacgccacctcccctgcagccgagtgcacgcagcagcctggcgtgacaagtgggcgacgccggggggcagggagccggggtccttggccctggccgggg

SEQ ID NO: 28 - DNA, 5844 bp, synthetic construct
  source 1..5844: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaaaatgaaggagttgagaagtacatactctgaggtttatcctaaataaactgaagtctaggcaagtggctgagctaggtttgccatgaatcaatctgctgagtatatttcttggtaactagttcatctttccttaattcagtcaacaaatatgatatcaagtttagtaagatgatgacagttgtcatgtaatactccatctggaagattgtatgcttctcagcttccctaacttttcttaatctaccttaagcttcctttctgaaaggtttctgtctttcctaacagtctcttctttccgccagctacctcttgtgtttaagaattaaaggaagatgagctaggaatcaagaaatacgcagaactgattcctgtcactggcgccaccctgccacttgcccagagctgtggtataaatttttttaataggctgaaataaaaatatgaatagcattttggtattaagcattaaattgataaaggctatgagatacacctgctctcaaccatgttaattttttattattggtattaataaattattacttactaacatattaacaagaattgcattgagaaacaaagcatccacaggccaaatcttctgaatttcaaatgtttatattaatgcgttgtattctagaaaagtagaattgtcttaagtagctttgtaatataaagtcatctatcagcccattacacctattagaatgttttaacctttatttcccacttttttgtttctagactgagtgtacttggtctggtatttgtggcataacttacggaacacataagaatgatacagatactatttaatgatgacctaatacaagcttagatagctaaggtgaaagcttctatggccttaacattttcctcttgaagaatgtattttctgtaataaaatacagtggctacttgaaatctataaacttatgtgaggtctggataaatctgagcaactttcttctttgtgctccaggaacctacgcactatatatataaataaagcttaagtaaacatcactgcaaaaaaaaaaaaaaaaa

SEQ ID NO: 29 - DNA, 5449 bp, synthetic construct
  source 1..5449: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaacaatccaattcctagatgctttctccagttatggccctgactgggatctccgaagaatgactcagctgaaactatttcatcattgcctacatttgtcttcagacgacctgcagtacaaacttggcttaaacctgcagttgacaatggattcaccgtgggagaataaattacaatggataaaaaacagcattacaatccctcaataaaatgggattttaacagacaaagctcctggtatgtggtcgttctcaggcccaaaggcccattttgcttttggctgcaactctgtaactctgtaacagagaccaagtgcctgcagggagcagatgtttggggcagtatctgctaaggtggctttgctgaaacttgtttctctcattgggtcttggaagtggcgaataggacgttcctatagaaaaactcagaaagagactgtgaaagataatcttaaagaggcactcacgaaacacaagctccttctccgggaaaaagaatcatcacccttgttatcccatgaaaattcatcttctccttttcagctgatatgaggacttgatctgtcctttcctataagtgccatctgtgtttaaatgaggctgttactctaactcaaagcagacttgcaagagtaaaataatcaatcttcatcata

SEQ ID NO: 30 - DNA, 5469 bp, synthetic construct
  source 1..5469: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaagagtgaaggagcaaaggggactcggccgccatgttaggagtactgggacgattccgcggagccgggcagaggttttaggggaatgattaacaaaggcgtccgaagaaatcgttgttggaaggtgaccaaggtggaaagagacgttgctttggccctgcaagtaagaagagagagggaatagcctgaaggagtaacactaaatttaaaatgacacttttttaccaaccagcgaaagcagatgttcaaaggggatattggccagagtctgtcacactaagatgagaaatgtcctttcttcctgaaggtgtctgatgtgtaaaaatatgatatactttgtgctgtttcctcccttcccttttgcatattattctgaaacaacattaactagttactttgcgtcattgaaggtatgcacttcccctctatgttaggagtgaataaaattaaaaatagatccttataacaaagaaaggcagatagaatgattaaaaatgaccaaaacatgttagaaacagtctctcaggtgtatgcagatggtaattacaaaaatactttttcaaaaaatgatcttctgtgtcatgtttctgggaacaagtcaagatgaatgagtttgatttttaagcagaagtagtatgtgttggtgtcatccatgaatacaacaatgaaaaaggc

SEQ ID NO: 31 - DNA, 5535 bp, synthetic construct
  source 1..5535: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttacccttctccagagcgccgtcgaccccacgccacgcagaggtgaagatacgcaggacgtcctctaaagcaacttccgtgggtgccaggttgtgggctgggggcggtgctgaccttgagagagcggaggaggcgcaggcgcagcacagtttaaacttgattttgtgaggggacgaacagcgtgacaattaaaagaagatctgggaatcatccaatgtgataatttatgtgaaatctttctgcaaactatacagtatgataaaaatataaggtagtttcactggaaacaacagaagactagaagctgatgtgacgtggcaatatcaaccaggaatacccatctcgagttctggatatgtgctctgagaaggtgcccatgtcaccgaccacactggaggccaatgcagatactgggggaaggttccatggtagctaagtgtggacaagctaatcactgaagttccctgatgcagagttgataatctctctagagttggatgaaagactgtgctgccttgaagctctgagagatgccatgccaattcaaggcagggtctttattgttgaaactcttgcgtaatgacatctttattcaggtgaaaatacaggatgaatttcaactatatgatattgtttatgttcctcagacatgttatttgtctttacaaagattggtttcaataaactgtgtgactgatattaaataaacatggaattttacacattcataataaaaaaaaaa

SEQ ID NO: 32 - DNA, 5639 bp, synthetic construct
  source 1..5639: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccagtcttttcgaggtaggagtcgactcctgtgaggtatggtgctgggtgcagatgcagtgtggctctggatagcaccttatggacagttgtgtccccaaggaaggatgagaatagctactgaagtcctaaagagcaagcctaactcaagccattggcacacaggcattagacagaaagctggaagttgaaatggtggagtccaacttgcctggaccagcttaatggttctgctcctggtaacgtttttatccatggatgacttgcttgggtaaggacatgaagacagttcctgtcataccttttaaaggtatggagagtcggcttgactacactgtgtggagcaagttttaaagaagcaaaggactcagaattcatgattgaagaaatgcaggcagacctgttatcctaaactagggtttttaatgaccacaacaagcaagcatgcagcttactgcttgaaagggtcttgcctcacccaagctagagtgcagtggcctttgaagcttactacagcctcaaacttctgggctcaagtgatcctcagcctcccagtggtctttgtagactgcctgatggagtctcatggcacaagaagattaaaacagtgtctccaattttaataaatttttgcaatccatcaaaaaaaaaaaaaaaaaatgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 33 - DNA, 6050 bp, synthetic construct
  source 1..6050: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccaggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattggtgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 34 - DNA, 6050 bp, synthetic construct
  source 1..6050: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccaccacagcctgctggaactttttacaaaatatgttgccatgctggaccctgggcactggacataagccccctggcagcctttttcatgtcacccaaaggggtaattgtcctactggtggtctgtaagatgagttagggtgacttgctaatagacattgtaaatcttaatatttatgtatgtattttattattaccggttttccatttatgatggtaatattgtttcttctaagaatatttatttttccttctaaatattgagataaaattcatgcttttgaaatgttctattcagtggcttttagtatatttgctatgttgtgcaaccatcgacactatccatttctagaactttttcgtcatcccaaacagacgctctgtattcataaaaaaataacttcctacctgtctctccccctagtctttggtaacctttgttatactggtaaactttgttgtgctctctgtctgtgtgaatttgcctattctaggggcctcatataagtgtaatcatacagtatttgtctttttgggtctgtctgatttcacttagcgggttttcagggttcattcatgttgcagcatataacagtactgcgttcctttttctggctgaataatattccactgtatggatagaccccattttgtttattcacacatcatttggacatttggattatttctggtttttggctattatgaacaatggtgctatgaacagttgcgtacaagtttttgtgtgaacatatgttttcaattctctcattatatacctaggagtagaattactgggtcatatggtaactgtatatttttgaggaactgccaaactattttcccacgtccatgcaccatttcacattcccaccagtaagtaagagggttccaatttctgcgcattcttgccaacactagttattatctgactttctggttataatcattctaatgagtgtgaagtagcctctggtgtcatttggatttgcatttctctgatgagtgatgctatcaagcacctttgctggtgctgttggccatatgtgtatgttccctggagaagtgtcttgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 35 - DNA, 5780 bp, synthetic construct
  source 1..5780: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccaagcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagtgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 36 - DNA, 5780 bp, synthetic construct
  source 1..5780: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaatgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 37 - DNA, 6031 bp, synthetic construct
  source 1..6031: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccattggccccactgtgcaccacacactcctttcccagcccaggggcacgcgaacaaaatgaggctcaagctgaccaggccgagccggaggaacgctggggcttggcagcagaagggatgggaccagagagaagggtgtggaggagaccccagtgagggccaggacatttcaggtaaagagaggtcatatctccgtacctcacttcctgacacaaacaagttttcactgttgtcagcaacaaagccctaatatagctgcggaagagaaaaactgcattgcattttgcctcctgcaagcatcatcaacagttactggaggaacgtaattccagaaagcttgaaagccgtggtgatggtaattatgtatcaaatgcctggttctatttctgttattattgttttgtcatttctgttttcccagcgatctgactgaactcgcagagggacaaatccagtttttctttttgacttttgtcaaactaaatcaggcctgatagaaaactcattgctctccggggaaacaaagtaggagccacgaaatgtcattttaacagagcgtgggtttggtgactgtaggaaaggatttgaggacgctccttctgttcggcttcctatgtcatgagcacaggctccacgcacgcacagacaccacggctcccggatgctgtggctccccgatcggggctcctgcagcgccagaagcccctccgggatgcttcgaggggctcccggtgggtggaggtacggacgccgctgcggccgccgccgccagtcctgctgctgttgttgctgctgcagtcacgtgggagcccctttaagtttccatagagaggcctctctggtgtcacatgatggacatgatataatgaaacaacattgtggagaggaaagcattaggggagcccacggctacaaaaacaagtgagtgagaagaggtgggaggaagagaaactacgccacctcccctgcagccgagtgcacgcagcagcctggcgtgacaagtgggcgacgccggggggcagggagccggggtccttggccctggccggggtgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 38 - DNA, 6031 bp, synthetic construct
  source 1..6031: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccaaaatgaaggagttgagaagtacatactctgaggtttatcctaaataaactgaagtctaggcaagtggctgagctaggtttgccatgaatcaatctgctgagtatatttcttggtaactagttcatctttccttaattcagtcaacaaatatgatatcaagtttagtaagatgatgacagttgtcatgtaatactccatctggaagattgtatgcttctcagcttccctaacttttcttaatctaccttaagcttcctttctgaaaggtttctgtctttcctaacagtctcttctttccgccagctacctcttgtgtttaagaattaaaggaagatgagctaggaatcaagaaatacgcagaactgattcctgtcactggcgccaccctgccacttgcccagagctgtggtataaatttttttaataggctgaaataaaaatatgaatagcattttggtattaagcattaaattgataaaggctatgagatacacctgctctcaaccatgttaattttttattattggtattaataaattattacttactaacatattaacaagaattgcattgagaaacaaagcatccacaggccaaatcttctgaatttcaaatgtttatattaatgcgttgtattctagaaaagtagaattgtcttaagtagctttgtaatataaagtcatctatcagcccattacacctattagaatgttttaacctttatttcccacttttttgtttctagactgagtgtacttggtctggtatttgtggcataacttacggaacacataagaatgatacagatactatttaatgatgacctaatacaagcttagatagctaaggtgaaagcttctatggccttaacattttcctcttgaagaatgtattttctgtaataaaatacagtggctacttgaaatctataaacttatgtgaggtctggataaatctgagcaactttcttctttgtgctccaggaacctacgcactatatatataaataaagcttaagtaaacatcactgcaaaaaaaaaaaaaaaaatgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 39 - DNA, 5636 bp, synthetic construct
  source 1..5636: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccaacaatccaattcctagatgctttctccagttatggccctgactgggatctccgaagaatgactcagctgaaactatttcatcattgcctacatttgtcttcagacgacctgcagtacaaacttggcttaaacctgcagttgacaatggattcaccgtgggagaataaattacaatggataaaaaacagcattacaatccctcaataaaatgggattttaacagacaaagctcctggtatgtggtcgttctcaggcccaaaggcccattttgcttttggctgcaactctgtaactctgtaacagagaccaagtgcctgcagggagcagatgtttggggcagtatctgctaaggtggctttgctgaaacttgtttctctcattgggtcttggaagtggcgaataggacgttcctatagaaaaactcagaaagagactgtgaaagataatcttaaagaggcactcacgaaacacaagctccttctccgggaaaaagaatcatcacccttgttatcccatgaaaattcatcttctccttttcagctgatatgaggacttgatctgtcctttcctataagtgccatctgtgtttaaatgaggctgttactctaactcaaagcagacttgcaagagtaaaataatcaatcttcatcatatgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 40 - DNA, 5656 bp, synthetic construct
  source 1..5656: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccaagagtgaaggagcaaaggggactcggccgccatgttaggagtactgggacgattccgcggagccgggcagaggttttaggggaatgattaacaaaggcgtccgaagaaatcgttgttggaaggtgaccaaggtggaaagagacgttgctttggccctgcaagtaagaagagagagggaatagcctgaaggagtaacactaaatttaaaatgacacttttttaccaaccagcgaaagcagatgttcaaaggggatattggccagagtctgtcacactaagatgagaaatgtcctttcttcctgaaggtgtctgatgtgtaaaaatatgatatactttgtgctgtttcctcccttcccttttgcatattattctgaaacaacattaactagttactttgcgtcattgaaggtatgcacttcccctctatgttaggagtgaataaaattaaaaatagatccttataacaaagaaaggcagatagaatgattaaaaatgaccaaaacatgttagaaacagtctctcaggtgtatgcagatggtaattacaaaaatactttttcaaaaaatgatcttctgtgtcatgtttctgggaacaagtcaagatgaatgagtttgatttttaagcagaagtagtatgtgttggtgtcatccatgaatacaacaatgaaaaaggctgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 41 - DNA, 5722 bp, synthetic construct
  source 1..5722: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttaccaaaggctcttctcagagccacccacccttctccagagcgccgtcgaccccacgccacgcagaggtgaagatacgcaggacgtcctctaaagcaacttccgtgggtgccaggttgtgggctgggggcggtgctgaccttgagagagcggaggaggcgcaggcgcagcacagtttaaacttgattttgtgaggggacgaacagcgtgacaattaaaagaagatctgggaatcatccaatgtgataatttatgtgaaatctttctgcaaactatacagtatgataaaaatataaggtagtttcactggaaacaacagaagactagaagctgatgtgacgtggcaatatcaaccaggaatacccatctcgagttctggatatgtgctctgagaaggtgcccatgtcaccgaccacactggaggccaatgcagatactgggggaaggttccatggtagctaagtgtggacaagctaatcactgaagttccctgatgcagagttgataatctctctagagttggatgaaagactgtgctgccttgaagctctgagagatgccatgccaattcaaggcagggtctttattgttgaaactcttgcgtaatgacatctttattcaggtgaaaatacaggatgaatttcaactatatgatattgtttatgttcctcagacatgttatttgtctttacaaagattggtttcaataaactgtgtgactgatattaaataaacatggaattttacacattcataataaaaaaaaaatgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 42 - DNA, 4796 bp, synthetic construct
  source 1..4796: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccattta

SEQ ID NO: 43 - DNA, 4957 bp, synthetic construct
  source 1..4957: mol_type = other DNA
gccaccatgcctgagcaggagagacagatcaccgcacgtgaaggagcttctcgaaagatcctgagcaaacttagcctgcctactagggcttgggaacccgctatgaagaagagttttgcatttgataacgtcggatacgaaggaggactcgatggtctgggaccgagttcacaagtggctacctcaactgtacggattttgggtatgacctgccaaagctgcgttaagagcattgaggatcggatatccaatcttaaaggtattattagcatgaaagtttctttggaacagggctcagcgactgttaaatacgtcccgagtgttgtttgtcttcagcaggtctgtcaccagataggcgatatgggcttcgaagcgtccatagctgaagggaaagcggcatcttggccgagcaggtctcttcccgcccaagaagccgtcgtcaagcttcgggttgagggtatgacatgtcagtcctgcgtgagctccatagaaggtaaagttcgcaagcttcagggcgtggtgcgggtcaaggtctccctcagtaatcaagaggccgtcataacataccagccatatcttattcaaccagaggacctgagggatcatgtaaacgacatgggctttgaagcggcaatcaagagtaaggtcgcgcctctttcccttggcccaatagacattgagcgcctccaaagtacgaacccgaaaaggccgttgagttctgccaaccaaaactttaataatagcgagactcttggccaccaagggtctcacgtagttacgttgcagctccgaatcgatggtatgcactgcaagtcctgtgtcctgaatattgaggagaatataggtcaacttcttggtgtacagtctatccaggtaagtcttgaaaataaaacggcccaggttaagtacgacccctcatgtacttcaccggtggcacttcaaagagcaatagaagccctgccgccagggaattttaaagtctctttgcctgatggggctgaaggctctggtacagatcacaggagctcttctagccatagcccgggttccccgccccgaaatcaggtgcaaggaacatgtagcacgaccctcatcgcaattgcgggtatgacttgtgcgagttgtgtacatagcatagagggaatgatatcccaattggaaggagttcaacaaattagcgtgagcctcgcggaggggacagccaccgtactctataaccccgccgtaatttcccctgaagagctgcgggccgcgatagaggatatgggattcgaagcctctgtagtatcagaaagttgttccacgaacccactcgggaaccattcagctggcaattctatggtacagacaaccgatggaacaccaaccagtcttcaagaagttgctcctcacactggacgactccccgcgaatcatgctcctgatatcttggctaagagcccccaatcaacacgggccgtcgctccacaaaagtgttttcttcaaatcaagggcatgacctgtgcatcatgcgtttccaatatagaaaggaatttgcagaaagaggccggggtactttctgtactcgttgctctcatggctggaaaagctgaaattaaatacgacccagaagtcatacagcctttggaaattgcccaattcatacaagatctcggattcgaagctgccgtaatggaggactatgccggcagcgatggtaatatcgagttgaccattaccggaatgacatgtgctagctgcgtacataatatcgagtccaagcttacgagaacgaacggaatcacatacgcttcagtagctctcgccacaagtaaggccctggtaaaatttgacccagagattattggacctagagacatcattaaaatcatcgaagaaatcggttttcatgcgagcctcgcgcagcgcaacccgaatgcgcaccatctggatcataagatggaaataaagcaatggaaaaaatcattcctttgcagcttggtattcggtatccccgtcatggctttgatgatctacatgctgatcccgtcaaacgagccacatcagagcatggtactcgatcataacattataccgggtcttagcatacttaatcttattttttttatcctttgcacattcgtccaacttttggggggttggtacttctacgtgcaggcgtataaatctctgcggcatcgcagcgccaatatggatgttttgatcgtattggctactagcattgcttatgtatattccctggtgatcctcgttgtggcggtggcggaaaaagcagagcggtcaccagtcacgttctttgatacgccaccaatgttgttcgtgttcattgcgctcggaaggtggttggagcaccttgcaaaatctaaaacaagtgaagccctggctaaacttatgtcacttcaggccacagaggccaccgttgttaccctcggtgaagacaaccttatcattagggaagaacaagttcccatggaactcgttcagcgaggagatatagttagggtggtccctgggggtaaatttcccgtcgacgggaaggttcttgagggaaacacgatggctgacgaaagcctcataactggggaagcgatgccggtgaccaaaaaacctgggagtacagtgatagcgggctctattaacgcacatggttctgtgcttattaaggctacacacgtaggtaacgacaccaccttggcacagatcgttaaacttgttgaggaggcccaaatgtctaaagcacctatacaacaactcgcggaccggttttcaggatactttgtaccctttattataataatgagcacactgactcttgtggtatggattgttatcggctttatcgatttcggagtcgtacaaaaatatttcccgaacccaaataagcacatcagtcagacggaagtcatcataaggtttgcttttcaaaccagcattacagtcctctgcatagcatgcccgtgttctctcggcctggccacacccacagcggtcatggtaggcaccggtgttgctgcccagaacggtatacttatcaaaggtggcaagccgttggaaatggctcataagatcaaaacggtgatgttcgataagactggaactatcacccatggggtccctcgtgttatgcgagttctgttgctgggcgatgttgccactctgccactcagaaaagttctcgccgtagtcggaactgccgaagcgtcaagcgaacaccccctgggtgtagcagtcactaaatactgtaaggaggagcttggcacagagacgctgggttactgtacagacttccaagccgttcctgggtgcggcatcggatgtaaggtgtcaaatgtagagggtatcctggctcactccgaacgaccccttagtgcgccggcctcccatcttaatgaagccggttctttgcctgctgagaaagacgcagcccctcagaccttttccgttctcataggcaatcgggaatggttgcggagaaacggcttgactattagcagtgatgtgtccgatgcaatgacggatcatgagatgaaaggtcaaaccgcgattctcgtggctatcgatggagtgctttgtggaatgatagcaatcgcagacgccgtcaaacaagaggcagccctcgccgtacacacactgcaatccatgggcgtggacgtcgttttgatcacgggcgataatcggaaaacggccagggccattgcaactcaagtagggataaacaaggtgtttgccgaagttctgcccagtcataaagtagctaaagttcaggaacttcagaacaaggggaagaaagtagccatggtgggggatggcgtcaacgactctccagcccttgcccaggctgacatgggcgtcgctatagggaccgggaccgacgtcgcaatcgaggcggcagacgtagtacttattaggaatgacttgctcgacgtagtggcatcaatccatctttcaaagcggactgtcagaaggatacggatcaacttggtactggcgctgatatacaacctcgtagggatacctatagctgcaggcgtcttcatgcctatcggcattgttctgcagccatggatgggatcagctgcgatggctgcgagtagcgtatccgtagtgctgtcctctctgcaacttaagtgctataagaaaccagatcttgaaaggtacgaggcgcaggcacacgggcacatgaaacccttgaccgcttctcaagttagtgtacacatcggtatggatgatcgctggcgggattcccctcgggctactccgtgggatcaagtcagttatgtgtctcaagtatcactttcttcacttacctctgacaagcccagccgccacagcgctgcagccgacgacgatggggacaagtggagtctcctgttgaatggtcgagacgaagaacaatatatcgactacaaagaccatgacggtgattataaagatcatgacatcgactataaggatgacgatgacaaaggctccggcgagggcaggggaagtcttctaacatgcggggacgtggaggaaaatcccggcccatcatcttcttcgaggatgacggcaactacaagtcgcgcgccgaggtaagagagctcgttgcgatattattacagcaacgaaaactgcaacggacctcccggggctaatgcggccgcggtagatctcatgttaagggttcttactataataaaataagataaataaataaataaatacatgctatcatatccctattagcctctacaaaaagaaaccaaacctcaaattttaaaaagaggtgtttataaatgacaaattgccatttatgttttgtgttttggctgggtttttccttgttcgcaccggacacctccagtgaccagacggcaaggtttttatcccagtgtatattaaaaagcggtcaggcagctaaaccaaaaggtttagcaattgcctctgatgagtcgctgaaatgcgacgaaaaccg

SEQ ID NO: 44 - DNA, 1067 bp, synthetic construct
  source 1..1067: mol_type = other DNA
ggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattgg

(19) United States
(12) Patent Application Publication
(10) Pub. No.: US 2025/0290067 A1
(43) Pub. Date: Sep. 18, 2025

(54) LOCALIZATION OF TRANS-SPLICING NUCLEIC ACID MOLECULES TO AND WITHIN THE CELLULAR NUCLEUS

(71) Applicant: Tacit Therapeutics, Inc., South San Francisco, CA (US)

(72) Inventor: David Allen NELLES, South San Francisco, CA (US)

(73) Assignee: Tacit Therapeutics, Inc., South San Francisco, CA (US)

(21) Appl. No.: 18/862,406

(22) PCT Filed: May 2, 2023
(86) PCT No.: PCT/US2023/066513

§ 371 Date: Nov. 1, 2024

Related U.S. Application Data
(60) Provisional application No. 63/337,793, filed on May 3, 2022.

(51) Int. Cl. C12N 15/113

(52) U.S. Cl. CPC C12N 15/113; C12N 2310/11; C12N 2320/33

(57) ABSTRACT

Disclosed are compositions comprising a nucleic acid encoding a localization domain. The localization domain may be configured to promote accumulation of the nucleic acid in the cellular nucleus as compared to a nucleic acid without the localization domain.

### CROSS-REFERENCE

This application claims the benefit of U.S. Provisional Application No. 63/337,793, filed May 3, 2022, which is entirely incorporated herein by reference.

### STATEMENT AS TO FEDERALLY SPONSORED RESEARCH

This invention was made with government support under Contract No. 2112383 awarded by National Science Foundation (NSF). The government has certain rights in the invention.

### INCORPORATION BY REFERENCE OF SEQUENCE LISTING

The present application is being filed along with a Sequence Listing in electronic format. The Sequence Listing is provided as a file entitled 63827-706601.XML, created Apr. 28, 2023, which is 177 kilobytes in size. The information in the electronic format of the Sequence Listing is incorporated by reference in its entirety.

### BACKGROUND

Various gene modification techniques may be used to modify and control an organism's genetic materials. These innovative techniques enable researchers to target specific genes and make precise alterations to, e.g., DNA, RNA, or protein, and offer new therapeutic strategies for a variety of disorders.

### SUMMARY

Recognized herein is a long-felt but unmet need in the art for the creation of efficacious treatments that address the underlying cause of human genetic diseases. Effective treatment of human genetic disease necessitates efficient replacement of defective genetic sequences in human cells. Recognized herein is the problem that RNA trans-splicing may not experience success due to low RNA editing efficiency and therefore low efficacy. The efficiency of RNA trans-splicing is defined as the fraction of a target RNA molecule that experiences a specific change in sequence composition that is mediated by trans-splicing. This efficiency measurement is a significant metric of therapeutic efficacy. The present disclosure provides compositions and methods for promoting the localization of trans-splicing nucleic acid therapeutics to the site of action in human cells. Such compositions and methods may increase efficiency of RNA trans-splicing. For example, one significant reason for inefficient trans-splicing is inefficient accumulation of RNA trans-splicing molecules at the site of action, since the spliceosome-mediated RNA trans-splicing requires binding and activity of endogenous cellular enzymes (e.g., spliceosome) that are unevenly distributed throughout the cellular nucleus. The present disclosure provides systems, compositions, and methods that transport therapeutic trans-splicing nucleic acid molecules to and within the cellular nucleus to increase the efficiency of RNA editing by the trans-splicing nucleic acid. The present disclosure also provides methods for replacement of chosen RNA sequences within target RNAs using RNA trans-splicing molecules to treat a disease in the context of a human gene therapy. The compositions as disclosed herein may comprise DNA or RNA encoding the replacement sequences and/or the sequences for nuclear localization. By inning RNA trans-splicing efficiency in this manner, the present disclosure describes a means to reverse human diseases via RNA editing with efficiency sufficient to reverse human diseases that currently lack effective treatments.

In some aspects, the present disclosure provides a composition comprising a nucleic acid encoding a localization domain configured to promote accumulation of the nucleic acid in the cellular nucleus as compared to a nucleic acid without the localization domain. In some embodiments, the composition further comprises an intronic domain configured to promote ribonucleic acid (RNA) splicing of the replacement domain.

In another aspect, the present disclosure provides a composition comprising a nucleic acid, comprising a sequence encoding: (a) a replacement domain that encodes a therapeutic sequence; (b) an intronic domain configured to promote ribonucleic acid (RNA) splicing of the replacement domain; (c) an antisense domain configured to promote binding to a target RNA molecule; and (d) a localization domain configured to promote accumulation of the nucleic acid in the cellular nucleus as compared to a nucleic acid without the localization domain. In some embodiments, the localization domain comprises a sequence configured to promote accumulation of the nucleic acid with nuclear speckles. In some embodiments, the localization domain configured to promote association of the trans-splicing nucleic acid with nuclear speckles is derived or isolated from a gene selected from the group consisting of MALAT1, NEAT1, MEG3, and XLOC_003520, GAS5, XLOC_009233, XLOC_004456, and PINT. In some embodiments, the localization domain encodes a sequence derived or isolated from a long non-coding RNA that is involved in transcriptional regulation. In some embodiments, the localization domain encodes a sequence derived or isolated from a long non-coding RNA that is involved in splicing regulation. In some embodiments, the localization domain encodes a sequence derived or isolated from a gene selected from the group consisting of: JPX, PVT1, NR2F1, and EMX2OS. In some embodiments, the localization domain encodes a sequence configured to promote association of the nucleic acid with the cellular transcriptional machinery. In some embodiments, the localization domain configured to promote association with the cellular transcriptional machinery is derived or isolated from a B2 long non-coding RNA. In some embodiments, the localization domain configured to promote association with the cellular transcriptional machinery is derived or isolated from a gene comprising short interspersed nuclear elements. In some embodiments, the localization domain encodes a sequence configured to promote association of the nucleic acid with nuclear paraspeckles. In some embodiments, the localization domain configured to promote association of the trans-splicing nucleic acid with nuclear speckles in derived or isolated from the gene NEAT1. In some embodiments, the localization domain encodes a sequence that associate with a splicing factor. In some embodiments, the localization domain encodes a sequence configured to promote accumulation of the trans-splicing nucleic acid in the cellular nucleus. In some embodiments, the localization domain configured to promote accumulation of the trans-splicing nucleic acid in the cellular nucleus is derived or isolated from a long noncoding RNA. In some embodiments, the long non-coding RNA is selected from the group consisting of MALAT1, NEAT1, MEG3, and XLOC_003526. In some embodiments, the localization domain is less than 300 bases from the 3′ end of the nucleic acid. In some embodiments, the localization domain is less than 300 bases from the 5′ end of the nucleic acid. In some embodiments, a tram-splicing molecule comprises 2 or more localization domains. In some embodiments, the composition further comprises a 3′ untranslated region that increases the stability of the trans-splicing molecule. In some embodiments, the composition further comprises a 5′ untranslated region that increases the stability of the trans-splicing molecule. In some embodiments, the replacement sequence comprises a gene expression-enhancing element. In some embodiments, the gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, the composition further comprises an RNA-binding protein that strengthens the interaction among the trans-splicing nucleic acid molecule and the target RNA molecule and increases trans-splicing efficiency. In some embodiments, the trans-splicing nucleic acid is RNA, DNA, a DNA/RNA hybrid, a nucleic acid analog, a chemically-modified nucleic acid, or a chimera composed of two or more nucleic acids or nucleic acid analogs. In some embodiments, the nucleic acid molecule further comprises a heterologous promoter. In some embodiments, the nucleic acid further encodes an enzyme staple molecule (ESM) domain configured to enhance a trans-splicing of the nucleic acid. In some embodiments, the ESM domain comprises a sequence encoding an engineered small nuclear RNA (snRNA) or portion thereof. In some embodiments, the snRNA or portion thereof the engineered small nuclear RNA molecule is derived or isolated from a human small nuclear RNA gene selected from the group consisting of: U1, U2, U4, U5, U6, U7, U11, and U12. In some embodiments, the engineered small nuclear RNA molecule is derived or isolated from a U1 small nuclear RNA gene or variant of the U1 small nuclear RNA gene. In some embodiments, the intronic domain further comprises one or more sequences configured to enhance the trans-splicing of the replacement domain. In some embodiments, the one or more sequences configured to enhance the trans-splicing of the replacement domain comprises a trans-splicing enhancer sequence. In some embodiments, the one or more sequences configured to enhance the trans-splicing of the replacement domain comprise a sequence having the formula X₁X₂X₃X₄X₅X₆ wherein; X₁ is selected from the group including adenine (A), uracil (U) and guanine (G); X₂ is selected from the group including adenine (A), uracil (U) and guanine (G); X₃ is selected from the group including adenine (A), uracil (U) and guanine (G); X₄ is selected from the group including adenine (A), uracil (U), cytosine (C) and guanine (G); X₅ is selected from the group including adenine (A), cytosine (C), uracil (U) and guanine (G); and X₆ is selected from the group including adenine (A), uracil (U) and guanine (G). In some embodiments, the one or more sequences configured to enhance the trans-splicing of the replacement domain comprise a sequence having the formula X₁X₂X₃X₄X₅X₆ wherein; X₁ is selected from the group including adenine (A), uracil (U) and guanine (G); X₂ is selected from the group including adenine (A), uracil (U) and guanine (G); X₃ is selected from the group including adenine (A), uracil (U) and guanine (G): X₄ is selected from the group including adenine (A), uracil (U) and guanine (G); X₅ is selected from the group including adenine (A), uracil (U) and guanine (G); and X₆ is selected from the group including uracil (U) and guanine (G). In some embodiments, the one or more sequence configured to enhance the trans-splicing of the replacement domain comprise a sequence having the formula X₁X₂X₃X₄X₅X₆ wherein; X₁ is selected from the group including adenine (A), uracil (U) and guanine (G); X₂ is selected from the group including uracil (U) and guanine (G); X₃ is selected from the group including adenine (A), uracil (U) and guanine (G); X₄ is selected from the group including uracil (U) and guanine (G); X₄ is selected from the group including uracil (U) and guanine (G); and X₅ is selected from the group including uracil (U) and guanine (G). In some embodiments, a sequence of said nucleic acid molecule encodes (i) an exonic sequence or portion thereof of a target ribonucleic acid (RNA) sequence and (ii) a localization domain configured to promote accumulation of the exonic sequence in a cellular nucleus as compared to a nucleic acid without the localization domain. In some embodiments, the localization domain comprises a sequence configured to promote accumulation of the nucleic acid with nuclear speckles. In some embodiments, the localization domain configured to promote association of the trans-splicing nucleic acid with nuclear speckles is derived or isolated from a gene selected from the group consisting of: MALAT1, NEAT1, MEG3, and XLOC_003526, GAS5, XLOC_009233, XLOC_004456, and PINT. In some embodiments, the localization domain encodes a sequence that is derived or isolated from a gene selected from the group consisting of: JPX, PVT1, NR2F1, and EMX2GS. In some embodiments, the localization domain encodes a sequence configured to promote association of the nucleic acid with the cellular transcriptional machinery. In some embodiments, the localization domain configured to promote association with the cellular transcriptional machinery is derived or isolated from a 82 long non-coding RNA. In some embodiments, the localization domain configured to promote association with the cellular transcriptional machinery is derived or isolated from a gene comprising short interspersed nuclear elements. In some embodiments, the localization domain encodes a sequence configured to promote association of the nucleic acid with nuclear paraspeckles. In some embodiments, the localization domain configured to promote association of the trans-splicing nucleic acid with nuclear speckles in derived or isolated from the gene NEAT1. In some embodiments, the localization domain encodes a sequence that associate; with a splicing factor. In some embodiments, the localization domain encodes a sequence configured to promote accumulation of the trans-splicing nucleic acid in the cellular nucleus. In some embodiments, the localization domain configured to promote accumulation of the trans-splicing nucleic acid in the cellular nucleus is derived or isolated from a long noncoding RNA. In some embodiments, the long non-coding RNA is selected from the group consisting of MALAT1, NEAT1, MEG3, and XLOC_003526. In some embodiments, the localization domain is less than 300 bases from the 3′ end of the nucleic acid. In some embodiments, the localization domain is less than 300 bases from the 5′ end of the nucleic acid. In some embodiments, trans-splicing molecule comprises 2 or more localization domains. In some embodiments, the composition further comprises a 3′ untranslated region that increases the stability of the trans-splicing molecule. In some embodiments, the composition further comprises a 5′ untranslated region that increases the stability of the trans-splicing molecule. In some embodiments, the replacement sequence comprises a gene expression-enhancing element. In some embodiments, the gene expression-enhancing element comprises a sequence derived or isolated from the group consisting of Woodchuck Hepatitis Virus (WHV) Posttranscriptional Regulatory Element (WPRE), triplex from MALAT1, the PRE of Hepatitis B virus (HPRE), and an iron response element. In some embodiments, the composition further comprises an RNA-binding protein that strengthens the interaction among the trans-splicing nucleic acid molecule and the target RNA molecule and increases trans-splicing efficiency. In some embodiments, the trans-splicing nucleic acid is RNA, DNA, a DNA-RNA hybrid, a nucleic acid analog, a chemically-modified nucleic acid, or a chimera composed of two or more nucleic acids or nucleic acid analogs. In some embodiments, the nucleic acid molecule further comprises a heterologous promoter. In some embodiments, the nucleic acid is engineered.

In another aspect, the present disclosure provides a vector comprising any of the compositions disclosed herein. In some embodiments, the vector is selected from the group consisting of adeno-associated virus, retrovirus, lentivirus, adenovirus, nanoparticle, micelle, liposome, lipoplex, polymersome, polyplex, and dendrimer.

In another aspect, the present disclosure provides a cell comprising any of the vectors disclosed herein.

In another aspect, the present disclosure provides method for treating a disease comprising administering to a patient in need thereof a therapeutically effective amount of any of the compositions disclosed herein, any of the vectors disclosed herein, or any of the cells disclosed herein.

In another aspect, the present disclosure provides a method for correcting a genetic defect in a subject comprising administering to a patient in need of a therapeutically effective amount of any of the compositions disclosed herein, any of the vectors disclosed herein, or any of the cells disclosed herein.

In another aspect, the present disclosure provides a method comprising administering a nucleic acid molecule to a cell, wherein said nucleic acid molecule encodes (i) a Replacement Domain that comprises an exonic sequence and (ii) a Localization Domain configured to promote accumulation of the exonic sequence in a cellular nucleus as compared to a nucleic acid without the one or more Localization Domains. In some embodiments, the cell is a human cell. In some embodiments, the administering the nucleic acid molecule to the cell comprises administering a vector comprising the nucleic acid molecule to the cell. In some embodiments, the vector is selected from the group consisting of a viral vector, of a nanoparticle, a micelle, a liposome or lipoplex, a polymersome, a polyplex, an exosome, and a dendrimer. In some embodiments, the viral vector is selected from the group consisting of a retrovirus, a lentivirus, an adenovirus, and an adeno-associated virus. In some embodiments, the cell comprises a target RNA comprising a target sequence. In some embodiments, the administering the nucleic acid molecule to the cell results in the target sequence being replaced by the exonic sequence of the Replacement Domain. In some embodiments, the target RNA is located in the cellular nucleus. In some embodiments, the method further comprises providing an RNA-binding protein that strengthens the interaction among the nucleic acid and the target RNA molecule, further wherein the RNA-binding protein is configured to increase a trans-splicing efficiency associated with a replacement of the target sequence with the exonic sequence. In some embodiments, the Localization Domain encodes a sequence configured to promote accumulation of the nucleic acid with nuclear speckles. In some embodiments, the Localization Domain configured to promote association of the nucleic acid with nuclear speckles is derived or isolated from a gene selected from the group consisting of: MALAT1, NEAT1, MEG3, and XLOC_003526, GAS5, XLOC_009233, XLOC_004456, and PINT. In some embodiments, the Localization Domain encodes a sequence that is derived or isolated from a gene selected from the group consisting of: JPX, PVT 1, NR2F1, and EMX2OS. In some embodiments, the Localization Domain encodes a sequence that promote association of the nucleic acid with the cellular transcriptional machinery. In some embodiments, the Localization Domain configured to promote association with the cellular transcriptional machinery is derived or isolated from a B2 long non-coding RNA. In some embodiments, the Localization Domain configured to promote association with the cellular transcriptional machinery is derived or isolated from a gene comprising short interspersed nuclear elements. In some embodiments, the Localization Domain encodes a sequence configured to promote association of the nucleic acid with nuclear paraspeckles. In some embodiments, the Localization Domain configured to promote association of the nucleic acid with nuclear speckles in derived or isolated from the gene NEAT1. In some embodiments, the Localization Domain encodes a sequence that associate with a splicing factor. In some embodiments, the Localization Domain encodes a sequence configured to promote accumulation of the nucleic acid in the cellular nucleus. In some embodiments, the Localization Domain configured to promote accumulation of the nucleic acid in the cellular nucleus is derived or isolated from a long noncoding RNA. In some embodiments, the long non-coding RNA is selected from the group consisting of: MALAT1, NEAT1, MEG3, and XLOC_003526. In some embodiments, the Localization Domain is less than 300 bases from the 3′ end of the nucleic acid. In some embodiments, the Localization Domain is less than 300 bases from the 5′ end of the nucleic acid. In some embodiments, the nucleic acid comprises 2 or more Localization Domains. In some embodiments, the method further comprises a 3′ untranslated region that increases the stability of the nucleic acid. In some embodiments, the method further comprises a 5′ untranslated region that increases the stability of the nucleic acid. In some embodiments, the replacement sequence comprises a gene expression-enhancing element. In some embodiments, the gene expression-enhancing element comprises a sequence derived or isolated from the group consisting of: Woodchuck Hepatitis Virus (WHV) Posttranscriptional Regulatory Element (WPRE), triplex from MALAT1, the PRE of Hepatitis B virus (HPRE), and an iron response element. In some embodiments, the nucleic acid is RNA, DNA, a DNA/RNA hybrid, a nucleic acid analog, a chemically-modified nucleic acid, or a chimera composed of two or more nucleic acids or nucleic acid analogs. In some embodiments, the nucleic acid further encodes an enzyme staple molecule (ESM) domain configured to enhance the trans-splicing of the replacement domain. In some embodiments, the ESM domain comprises a sequence encoding an engineered small nuclear RNA (snRNA) or portion thereof. In some embodiments, the snRNA or portion thereof the engineered small nuclear RNA molecule is derived or isolated from a human small nuclear RNA gene chosen from a group consisting of U1, U2, U4, U5, U6, U7, U11, and U12. In some embodiments, the engineered small nuclear RNA molecule is derived or isolated from a U1 small nuclear RNA gene or variant of the U1 small nuclear RNA gene. In some embodiments, the nucleic acid further encodes an intronic domain. In some embodiments, the intronic domain further comprises one or more sequences configured to enhance the trans-splicing of the replacement domain. In some embodiments, the one or more sequences configured to enhance the trans-splicing of the replacement domain comprises a trans-splicing enhancer sequence. In some embodiments, the one or more sequences configured to enhance the tram-splicing of the replacement domain comprise a sequence having the formula X₁X₂X₃X₄X₅X₆ wherein; X₁ is selected from the group including adenine (A), uracil (U) and guanine (G); X₁ is selected from the group including adenine (A), uracil (U) and guanine (G); X₂ is selected from the group including adenine (A), uracil (U) and guanine (G); X₃ is selected from the group including adenine (A), uracil (U), cytosine (C) and guanine (G); X₄ is selected from the group including adenine (A), cytosine (C), uracil (U) and guanine (G); and X₅ is selected from the group including adenine (A), uracil (U) and guanine (G). In some embodiments, the one or more sequences configured to enhance the trans-splicing of the replacement domain comprise a sequence having the formula X₁X₂X₃X₄X₅X₆ wherein; X₁ is selected from the group including adenine (A), uracil (U) and guanine (G): X₂ is selected from the group including adenine (A), uracil (U) and guanine (G); X₃ is selected from the group including adenine (A), uracil (U) and guanine (G); X₄ is selected from the group including adenine (A), uracil (U) and guanine (G); X₅ is selected from the group including adenine (A), uracil (U) and guanine (G); and X₆ is selected from the group including uracil (U) and guanine (G). In some embodiments, the one or more sequences configured to enhance the trans-splicing of the replacement domain comprise a sequence having the formula X₁X₂X₃X₄X₅X₆ wherein; X₁ is selected from the group including adenine (A), uracil (U) and guanine (G); X₂ is selected from the group including uracil (U) and guanine (G); X₃ is selected from the group including adenine (A), uracil (U) and guanine (G); X₄ is selected from the group including uracil (U) and guanine (G): X₅ is selected from the group including uracil (U) and guanine (G); and X₆ is selected from the group including uracil (U) and guanine (G).

### INCORPORATION BY REFERENCE

All publications, patents, and patent applications mentioned in this specification are herein incorporated by reference to the same extent as if each individual publication, patent, or patent application was specifically and individually indicated to be incorporated by reference. Further, to the extent that any meaning or definition of a term in this document conflicts with any meaning or definition of the same term in a document incorporated by reference, the meaning or definition assigned to that term in this document shall govern.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 illustrates the unmet need addressed by the systems and methods described herein and provides a schematic of said systems and methods of the present disclosure. FIG. 1A schematically illustrates an example of a concept of human genetic disease where mutated (“defective”) DNA sequences are transcribed into RNA which directly contribute to disease (“RNA pathogenicity”) or are translated into disease-causing protein (“translation of pathogenic protein”). FIG. 1B illustrates an example of a concept of RNA trans-splicing technology where a mutation-carrying RNA molecule is targeted by a trans-splicing nucleic acid that corrects the mutation. This low efficiency is associated with trans-splicing activity is typically insufficient to halt or reverse progression of disease. FIG. 1C further schematically illustrates state-of-the-art trans-splicing technology where the trans-splicing nucleic acid is localized to the cytoplasm or locations in the cellular nucleus rather than the site of RNA splicing. As a result, RNA trans-splicing occurs with low efficiency.

FIG. 2 illustrates the mechanisms by which the systems and methods described herein can increase RNA trans-splicing efficiency via localization of the trans-splicing nucleic acid to specific locations within the cell. FIG. 2A schematically illustrates the concept of localization sequences that promote the accumulation of the trans-splicing nucleic acid in the cellular nucleus. As RNA splicing occurs primarily in the nucleus, this accumulation of the RNA trans-splicing molecule in the nucleus increases RNA trans-splicing efficiency. As a result, the levels of “corrected RNA” and therapeutic efficacy are increased. FIG. 2B illustrates the concept of localization sequences that promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. As a result, the levels of “corrected RNA” and therapeutic efficacy are increased. FIG. 2C illustrates the concept of localization sequences that promote the accumulation of the trans-splicing nucleic acid to nuclear speckles and paraspeckles. As RNA splicing occurs in and around nuclear speckles and paraspeckles, this accumulation of the RNA trans-splicing molecule to speckles and paraspeckles increases RNA trans-splicing efficiency. As a result, the levels of “corrected RNA” and therapeutic efficacy are increased.

FIG. 3 illustrates three embodiments of the trans-splicing nucleic acid described in this disclosure. FIG. 3A describes a double trans-splicing molecule which carries two antisense domains, one replacement domain, two intronic domains, and at least one Localization Domain at the 5′ and/or 3′ end of the trans-splicing molecule. This design promotes replacement of an internal sequence within the target RNA while maintaining the adjacent 5′ and 3′ sequences around the replaced sequence. FIGS. 3B and 3C describe terminal trans-splicing molecules that both comprise one antisense domain, one replacement domain, one intronic domain, and at least one localization Domain at the 5′ and/or 3′ end of the trans-splicing molecule. FIG. 3B illustrates the design of a 3′ terminal trans-splicing nucleic acid that will replace the 3′ terminal end of a target RNA while maintaining the 5′ end. FIG. 3C illustrates the design of a 5′ terminal trans-splicing molecule that will replace the 5′ terminal end of a target RNA while maintaining the 3′ end.

FIG. 4 illustrates an experiment designed to reveal the importance of localization sequences in the context of internal trans-splicing via production of GFP protein. FIG. 4A illustrates the design of a split GFP reporter that carries N- and C-terminal portions of GFP (“N-GFP” and “C-GFP”) but lacks an internal GFP sequence required for fluorescence. In the reporter, this internal sequence is replaced by a short exon with a stop colon that is flanked by introns. The internal sequence (“int-GFP”) is the replacement sequence within an RNA trans-splicing molecule that is flanked by two intronic sequences, two antisense sequences, and one or more localization sequences. FIG. 4B illustrates the activity of the reporter alone so that cis-splicing produces a GFP sequence interrupted by a stop codon therefore producing no GFP signal. FIG. 4C illustrates the activity of the reporter in the presence of the trans-splicing molecule without inclusion of localization sequences in the trans-splicing molecule so that similarly cis-splicing occurs primarily and GFP signal is not efficiently generated. This is because localization sequences that promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the lack of nuclear localization sequences may result in less accumulation of trans-slicing RNA to the site of transcription, thereby resulting in lower trans-splicing efficiency. FIG. 4D illustrates the activity of the reporter in the presence of the trans-splicing molecule with inclusion of localization sequences so that trans-splicing occurs primarily and GFP signal is efficiently produced. Localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the inclusion of nuclear localization sequences may result in greater accumulation of trans-slicing RNA to the site of transcription, thereby resulting in greater trans-splicing efficiency.

FIG. 5 illustrates an experiment designed to reveal the importance of localization sequences in the context of 5′ terminal trans-splicing. FIG. 5A illustrates the design of a split GFP reporter that carries a C: terminal portion of GFP (“C-GFP”) but lacks an N-terminal GFP sequence required for fluorescence. In the reporter, this N-terminal GFP sequence is replaced by a short exon with a stop codon that is flanked by introns. The N-terminal sequence (“N-GFP”) is the replacement sequence within an RNA trans-splicing molecule that is flanked by one intronic sequence, one antisense sequence, and one or more and one or more localization sequences. FIG. 5B illustrates the activity of the reporter alone so that cis-splicing produces a GFP sequence interrupted by a stop codon therefore producing no GFP signal. FIG. 5C illustrates the activity of the reporter in the presence of the trans-splicing molecule without inclusion of localization sequences in the trans-splicing molecule so that similarly cis-splicing occurs primarily and GFP signal is not efficiently produced. The results of FIGS. 5B and SC occur, in part, because localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the lack of nuclear localization sequences may result in less accumulation of trans-slicing RNA to the site of transcription, thereby resulting in lower trans-splicing efficiency. FIG. 5D illustrates the activity of the reporter in the presence of the trans-splicing molecule with inclusion of localization sequences so that trans-splicing occurs primarily and GFP signal is efficiently produced. Localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the inclusion of nuclear localization sequences may result in greater accumulation of trans-slicing RNA to the site of transcription, thereby resulting in greater trans-splicing efficiency.

FIG. 6 illustrates an experiment designed to reveal the importance localization sequences in the context of 3′ terminal trans-splicing. FIG. 6A illustrates the design of a split GFP reporter that carries a N-terminal portion of GFP (“N-GFP”) but lacks an C-terminal GFP sequence required for fluorescence. In the reporter, this C-terminal GFP sequence is replaced by a short exon with a stop codon that is flanked by introns. The C-terminal sequence (“C-GFP”) is the replacement sequence within an RNA trans-splicing molecule that is flanked by one intronic sequence, one antisense sequence, and one or more and one or more localization sequences. FIG. 6B illustrates the activity of the reporter alone so that cis-splicing produces a GFP sequence interrupted by a stop codon therefore producing no GFP signal. FIG. 6C illustrates the activity of the reporter in the presence of the trans-splicing molecule without inclusion localization sequence in the trans-splicing molecule so that similarly cis-splicing occurs primarily and GFP signal is not efficiently produced. The results of FIGS. 6B and 6C occur, in part, because localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the lack of nuclear localization sequences may result in less accumulation of trans-slicing RNA to the site of transcription, thereby resulting in lower trans-splicing efficiency. FIG. 6D illustrates the activity of the reporter in the presence of the trans-splicing molecule with inclusion of localization sequences so that trans-splicing occurs primarily and GFP signal is produced. Localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the inclusion of nuclear localization sequences may result in greater accumulation of trans-slicing RNA to the site of transcription, thereby resulting in greater trans-splicing efficiency.

FIG. 7 illustrates a concept whereby trans-splicing can be used in the context of a gene therapy to deliver a replacement gene. In this case, the replacement gene is ATP7B, a gene that is primarily expressed in the liver and mutated in Wilson's disease. By trans-splicing the ATP7B coding sequence into a liver-specific and highly-expressed gene such as ALB, the ATP7B gene expression can be generated in the liver only.

FIG. 8 describes the influence of various lncRNA sequences on the activity of a trans-splicing nucleic acid that targets the human ALB gene. Each bar represents a different trans-splicing molecule that is identical except for the addition of a human lncRNA sequence. The level of trans-spliced RNA product was assessed using RT-PCR with primers that target the trans-spliced product exclusively. The sequence of trans-splicing molecules P1779-P1802 are listed elsewhere.

FIGS. 9A-9B illustrate one example embodiment of the methods described herein. FIG. 9A illustrates a system composed of a donor RNA (e.g., a Replacement Domain encoding an exonic sequence that corresponds to a target RNA sequence or portion thereof) and an engineered small nuclear RNA (esnRNA). The combination of RNA donor molecule and esnRNA correct mutated RNAs via hybridization of the RNA donor to the target RNA carrying a mutation, followed by association of the esnRNA with the RNA donor, results in recruitment of spliceosome components and trans-splicing among the RNA donor molecule and the target RNA. This yields a corrected target RNA with the RNA donor molecule replacing a chosen sequence in the target RNA. FIG. 9B illustrates the how the components interact. Base pairing among the RNA donor and target RNA bring these molecule in close proximity. Base pairing among the esnRNA and the RNA donor brings spliceosome components in close proximity which promotes a trans-splicing reaction among the target RNA and the RNA donor.

FIG. 10 illustrates three example embodiments of the compositions and methods described in this disclosure. FIG. 10A describes a double trans-splicing molecule which carries two antisense domains, one replacement domain, two intronic domains, and at least two trans-splicing enhancer sequences within the intronic domains. This design promotes replacement of an internal sequence within the target RNA while maintaining the adjacent 5′ and 3′ sequences around the replaced sequence. FIGS. 2B and 2C describe terminal trans-splicing molecules that both contain one antisense domain, one replacement domain, one intronic domain, and at least one trans-splicing enhancer sequence within the intronic domain. FIG. 10B illustrates the design of a 3′ terminal trans-splicing RNA that will replace the 3′ terminal end of a target RNA while maintaining the 5′ end. FIG. 10C illustrates the design of a 5′ terminal trans-splicing molecule that will replace the 5′ terminal end of a target RNA while maintaining the 3′ end.

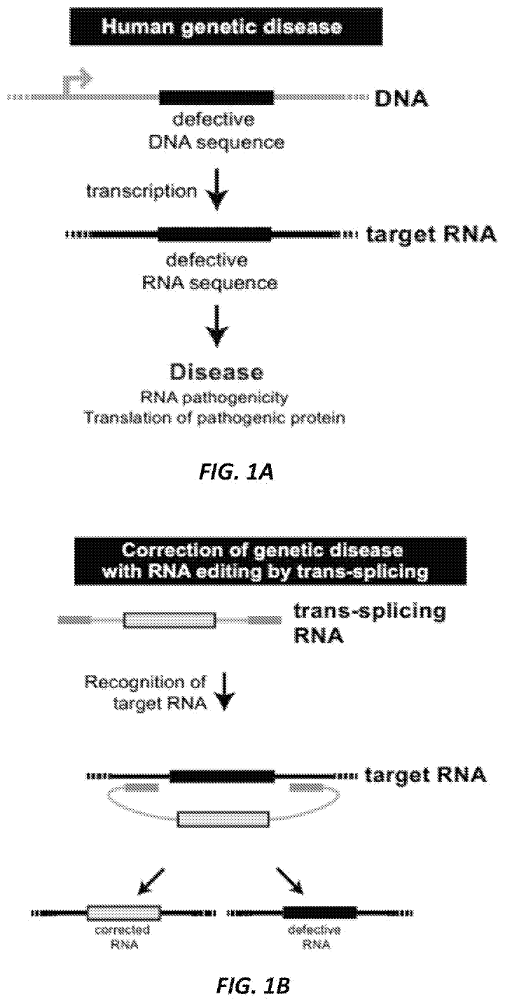

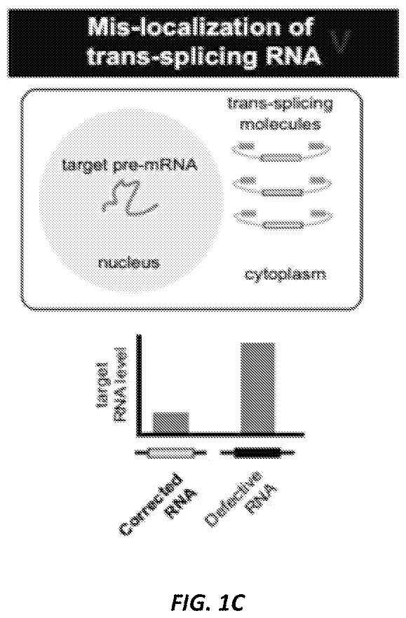

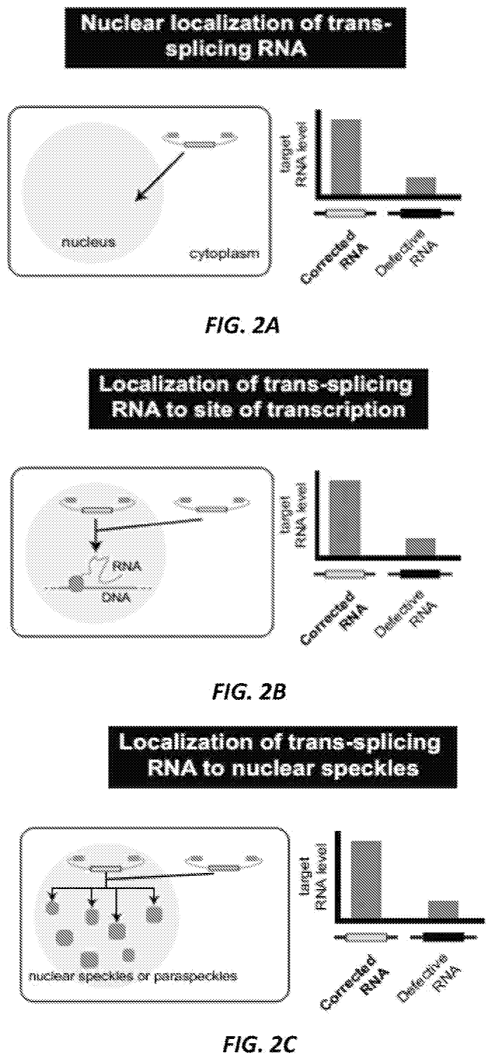

### DETAILED DESCRIPTION

The present disclosure provides compositions and methods for trans-splicing. As splicing in eukaryotes occurs primarily in the nucleus, provided herein are nucleic acids encoding a Nuclear Localization Domain comprising one or more sequences that promote nuclear localization. The nucleic acid may be engineered. A Nuclear Localization Domain may also be known as a Localization Domain, or the like. The nucleic acids may further encode a Replacement Domain encoding an exonic sequence that corresponds to a target RNA sequence or portion thereof. The target RNA sequence or portion thereof may comprise a missing or mutated sequence. The exonic sequence may be trans-spliced to the target RNA or portion thereof, thereby correcting the target RNA sequence. The localization sequence may promote the accumulation of the exonic sequence to the target RNA, and may thereby promote trans-splicing. The nucleic acid molecule can comprise a ribonucleic acid (RNA), a deoxyribonucleic acid (DNA), or any combination thereof. The nucleic acid molecule comprising DNA may be transcribed into RNA. In some embodiments, an RNA molecule that carries localization sequences can selectively bind to and promote a trans-splicing reaction with a target RNA molecule. The nucleic acid may be engineered.

The present disclosure provides, in some embodiments, a composition comprising a trans-splicing nucleic acid molecule comprising (a) at least one domain that promotes trans-splicing (“Intronic Domain”), (b) at least one binding domain (“Antisense Domain”) that comprises a sequence complementary to a pre-mRNA present in a human cells (“Target RNA”), (c) a coding domain that is inserted into the Target RNA via trans-splicing (“Replacement Domain”), and (d) a localization sequence (“Localization Domain”) that promotes transport of the trans-splicing molecule to the cellular nucleus or specific locations within the cellular nucleus.

The Localization Domain may promote transport of the trans-splicing molecule to or within the cellular nucleus which results in accumulation of the trans-splicing molecule at the site of action and therefore increases the efficiency of the trans-splicing reaction. In some embodiments, the Localization Domain can promote the transport of the tram-splicing molecule to or within the cellular nucleus, resulting in an accumulation of the trans-splicing molecules at the site of action and thereby increasing the efficiency of the trans-splicing reaction. In some embodiments, the Localization Domain can promote the transport of the trans-splicing nucleic acid to the cellular nucleus or to specific locations within the cellular nucleus. In some embodiments, Localization Domain can comprise sequences that bind to enzymes involved in transcription (such as polymerase II or transcription-associated enzymes), RNA splicing, or the formation of nuclear speckles. In some embodiments, systems, methods composition described herein can promote RNA trans-splicing, wherein the RNA trans-splicing is mediated by the cellular spliceosome. In some embodiments, as the components of the spliceosome are located inside and within the cellular nucleus, the Localization Domain can increase RNA trans-splicing activity by promoting accumulation of the RNA trans-splicing molecule to the location of the spliceosome. In some embodiments, an DNA molecule can carry localization sequences. In some embodiments, the localization sequences carried by the DNA molecule encode RNA localization sequences. In some embodiments, the DNA molecule can encode a gene or portion thereof to be transcribed.

In some embodiments of the compositions of the disclosure, the sequence encoding the trans-splicing nucleic acid further comprises a sequence encoding a promoter capable of expressing the trans-splicing nucleic acid in a eukaryotic cell.

In some embodiments of the compositions of the disclosure, the eukaryotic cell is an animal cell. In some embodiments, the animal cell is a mammalian cell. In some embodiments, the animal cell is a human cell.

In some embodiments of the compositions and methods of the disclosure, a vector comprises the nucleic acid molecule a % disclosed herein. The vector may be a viral vector.

The disclosure provides an RNA molecule that carries localization sequences that selectively binds to and promotes a trans-splicing reaction with a target RNA molecule. An aspect of the present disclosure provides a composition comprising a trans-splicing nucleic acid molecule comprise (a) at least one domain that promotes trans-splicing (e.g., Intronic Domain), (b) at least one binding domain (e.g., Antisense Domain) comprising a sequence complementary to a pre-mRNA of a human cells (e.g., Target RNA), (c) a coding domain that is inserted into the Target RNA via trans-splicing (e.g., Replacement Domain), and (d) a localization sequence (e.g., Localization Domain) that promotes transport of the trans-splicing molecule to the cellular nucleus or specific locations within the cellular nucleus.

In other embodiments, the systems, methods and compositions described herein can provide a nucleic acid sequence encoding the trans-splicing nucleic acid molecule. In some embodiments, the trans-splicing nucleic acid molecule can carry a Replacement Domain that corresponds to a mutated or missing sequence in a target RNA. In some embodiments, the Replacement Domain can correspond to a mutated or missing sequence in a target RNA. In some embodiments, a nucleic acid encoding the trans-splicing nucleic acid molecules can be DNA. In some embodiments, a nucleic acid encoding the trans-splicing nucleic acid molecule can be RNA. In some embodiments, the DNA molecule is transcribed into a messenger RNA molecule, and the messenger RNA molecule can then selectively bind and promote a trans-splicing reaction with a target RNA. In some embodiments, disclosure provides vectors, compositions and cells comprising or encoding the trans-splicing nucleic acid molecules. The present disclosure provides methods of using the trans-splicing nucleic acid molecule, vectors, compositions and cells to treat a disease or disorder.

In one aspect, the present disclosure provides a trans-splicing nucleic acid molecule comprising four types of domains. In a second aspect, the present disclosure provides a trans-splicing DNA molecule comprising four types of domains. In some embodiments, the trans-splicing DNA can comprise a gene or portion thereof to be transcribed. In some embodiments, the gene or portion thereof can correspond to a missing or mutated sequence in a target RNA. In some embodiments, the DNA molecule can be transcribed into a messenger RNA molecule, and the messenger RNA molecule can then selectively bind and promote a trans-splicing reaction with a target RNA. In some embodiments, one of the four domain types may comprise the Replacement Domain, which can be inserted into a Target RNA molecule via a trans-splicing reaction. In some embodiments, a DNA molecule can comprise a gene or portion thereof encoding the Replacement Domain described herein. In some embodiments, an RNA molecule can comprise the Replacement Domain described herein. In some embodiments, a second domain type can be the Antisense Domain which is complementary to a Target RNA. In some embodiments, a DNA molecule can comprise an Antisense Domain described herein. In some embodiments, an RNA molecule can comprise an Antisense Domain described herein. In some embodiments, a third domain type can be the Intronic Domain which promotes the trans-splicing reaction between the trans-splicing nucleic acid molecule and the Target RNA. The Intronic Domain can comprise RNA. The intronic Domain can comprise DNA. The intronic Domain comprising DNA can be transcribed into an Intronic Domain comprising RNA. In some embodiments, an DNA molecule can comprise an Intronic Domain described herein. In some embodiments, an RNA molecule can comprise an Intronic Domain described herein. In some embodiments, the Intronic Domain can promote the trans-splicing reaction between the trans-splicing DNA molecule and the target RNA. In some embodiments, the fourth domain can be a Localization Domain that carries sequences (e.g., Localization Sequence) that promote the accumulation of the trans-splicing molecule to and within the cellular nucleus. In some embodiments, the Localization Domain can promote localization of trans-splicing molecules to the cellular nucleus from the cytoplasm or to specific structures within the nucleus such as nuclear speckles or paraspeckles. In some embodiments, the Localization Domain can promote association of the trans-splicing molecule with nuclear-localized proteins and protein complexes such as the spliceosome, transcriptional proteins, or splicing factors.

This combination of trans-splicing domains (Replacement, Intronic, and Antisense Domains) with the Localization Domain can promote RNA trans-splicing in a manner that is sufficient to replace disease-causing RNA sequences in human cells to address disease. Low efficiency may be a major barrier to many nucleic acid editing approaches including RNA trans-splicing. The present disclosure provides compositions and methods for specifically targeting disease-causing RNA molecules and replacing disease-causing RNA sequences within these RNA molecules with higher efficiency. The trans-splicing nucleic acid molecule implementations may demonstrate utility in a variety of contexts including replacement of disease-causing sequences or insertion of engineered sequences into Target RNAs.

The engineered sequences can alter the translation or stability of Target RNAs to increase or decrease protein production or Target RNA levels. The engineered sequences (e.g., polynucleotide sequences) disclosed herein can be colon-optimized. Codon optimization refers to the fact that different cells differ in their usage of particular codons. This codon bias can correspond to a bias in the relative abundance of particular tRNAs in the cell type. By altering the codons in the sequence to match with the relative abundance of corresponding tRNAs, it is possible to increase expression. In some instances, it is also possible to decrease expression by deliberately choosing codons for which the corresponding tRNAs are rare in a particular cell type.

In some instances. DNA can encode a localization sequence that can be translated into RNA in order to modify (e.g., fix) the sequence. In some instances, the modification or fixing of the RNA sequence via trans-splicing can increase protein production. In some embodiments, the systems, methods, and composition provided herein can comprise vectors and cells comprising or encoding the trans-splicing nucleic acid and methods of using the trans-splicing nucleic acid compositions.

In one aspect, described herein is an RNA technology that can enable replacement of arbitrary sequences within specific RNA molecules in living cells. In another aspect, described herein is a DNA technology that can enable replacement of arbitration sequences within specific RNA molecules in living cells. In some embodiments, the DNA molecule can encode a gene or portion thereof to be transcribed. The technology, based on RNA trans-splicing, can utilize the naturally-existing spliceosome in human cells to provide the catalytic activity for this trans-splicing process. Without being limited by theory. RNA splicing occurs within RNA molecules where exons are concatenated, and introns removed from immature messenger RNA molecules (pre-mRNAs) to form mature messenger RNA molecules (mRNAs). This process is referral to as cis-splicing and requires the set of enzymes and noncoding RNAs collectively known as the spliceosome. RNA trans-splicing is a process by which the spliceosome concatenates exons derived from distinct and separate RNA molecules. This process rarely occurs in human cells. The present disclosure provides for compositions that increase the efficiency of RNA trans-splicing. These improved RNA trans-splicing compositions can be used to replace mutated sequence within a target RNA molecule to address a human disease. Replacement of arbitrary RNA sequences is a general ability with innumerable specific applications a few of which have been explored as relevant demonstrations. RNA trans-splicing can insert engineered sequences into a target RNA to impart new activities to the target RNA such as altered RNA stability or altered RNA translation. This feature can be used to increase production of protein by a target RNA. In the broadest sense, this RNA trans-splicing technology can impart arbitrary changes to both coding and non-coding regions of target RNs.

### Compositions

### Localization Domain

The present disclosure provides nucleic acids encoding a Localization Domain. A Localization Domain may comprise one or more sequences. e.g., nuclear localization sequence, that may promote the accumulation of compositions as described herein in a cellular nucleus. In eukaryotes, the process of transcription takes place in a cellular nucleus. To that end, an increased accumulation of nucleic acids for trans-splicing to the nucleus may increase the occurrence of trans-splicing.

Compositions as described herein may comprise a nucleic acid encoding a localization sequence. The nucleic acid may comprise RNA. The RNA encoding the localization sequence may further encode: an exonic sequence corresponding to a target RNA. The localization sequence on the RNA may promote trans-splicing of the exonic sequence into the target RNA. The nucleic acid may comprise DNA encoding a localization sequence. The DNA encoding the localization sequence may be transcribed into RNA. The DNA may further encode an exonic sequence corresponding to a target RNA. The DNA encoding the exonic sequence may be transcribed into RNA. In this manner, a DNA molecule encoding the localization sequence and the exonic sequence may be transcribed into RNA, and the localization sequence on the RNA may promote trans-splicing of the exonic sequence into the target RNA. The trans-splicing of the exonic sequence into the RNA may treat, e.g., a mutation of the target RNA. A variety of RNA sequences placed in a heterologous context may promote the accumulation of RNAs in the nucleus or within specific structures in the nucleus such as nuclear speckles or paraspeckles. The present disclosure further assesses 1) whether the presence of localization sequences interferes with trans-splicing reactions, 2) which putative localization sequences function in the context of trans-splicing, and 3) whether the accumulation of trans-splicing molecules in specific locations increases RNA trans-splicing efficiency. As the activity of many known RNA localization sequences may be context-dependent, the present disclosure provides a distinct group of localization sequences that may function in the context of trans-splicing. This is confirmed by experiments that indicate that activity of localization in other contexts (i.e., outside of the scope of trans-splicing) is not necessarily predictive of activity in trans-splicing.

In some instances, a trans-splicing molecule provided herein can comprise localization sequences. In some instances, a trans-splicing molecule provided herein may not comprise localization sequences. In some embodiments, localization sequences that increase trans-splicing activity can also increase the levels of trans-splicing molecule. In some embodiments, a localization sequence described herein can be derived from mRNA, long noncoding RNAs, and synthetic sequences that can alter that localization of varied transcript types within the cellular nucleus. In some embodiments, a localization sequence described herein can function specifically within the context of trans-splicing. In some embodiments, a localization sequence described herein can function universally (e.g., any systems)

The Localization Domain may promote transport of the trans-splicing nucleic acid to the cellular nucleus or to specific locations within the cellular nucleus. The Localization Domain may comprise one or more localization sequences that bind to enzymes involved in transcription (such as polymerase 11 or transcription-associated enzymes). RNA splicing, or the formation of nuclear speckles. There exist various means to promote RNA trans-splicing and the present disclosure focuses on RNA trans-splicing that is mediated by the cellular spliceosome. As the components on the spliceosome may be located inside and within the cellular nucleus, the Localization Domain may increase RNA trans-splicing activity by promoting accumulation of the RNA trans-splicing molecule to the location of the spliceosome. In other embodiments, the present disclosure provides a composition comprising a nucleic acid sequence encoding the trans-splicing nucleic acid molecule.

In some embodiments, the Localization Domain can carry sequences that promote nuclear localization of the trans-splicing molecule and is derived or isolated from a gene selected from the group consisting of: CDKN2B-AS1 [NR_003529]; BANCR [NR_047671]; CASC15 [NR_015410]; CRNDE [NR_034105]; EMX2OS [NR_002791]; EVF2 [NR_015448]; FENDRR [NR_036444]; FIX [NR_028379]; GAS5 [NR_002578]; HOTAIR [NR_003716]; HOTAIRM1 [NR_038366]; HOXA-AS3 [NR_038832]; UOXA11-AS [NR_002795]; JPX [NR_024582]; LHX5-AS1 [NR_126425]; LINC01578 [NR_037600]; LINC00261 [NR_001558]; MALAT1 [NR_002819.4]; MEG3 [NR_046473]; TUNAR [NR_038861): MIAT [NR_033320]; NEAT1 (NR_028272]; NR2F1-AS1 [NR_021490]; LILAC-PINT [NR015431], PSMA3-AS1 [NR_029434]; EMX2OS [ENSG00000229847]; PVT1 [NR_003367]; MEG8 [NR_024149]; RMST [NR_024037]; SENCR [NR_038908]; SIX3-AS1 [NR_103786]; SOX21-AS1 [NR_046514]; TERC [NR_001566]; TUG1 [NR_002323]; XIST [NR_001564], malat1 [NR_002847.3], Nfx1 [NM_023739.3], Ogt [NM_139144.4], Nlrp6 [NM_133.946.2], Mlxip1 [NM_021455.5], Leng8 [NM_001374609.1], Gcgr [NM_008101.2], Gck [NM_001287386.1], Acly [NM_001199296.1], Ccnl1 [NM_001355433.1], Ccnl2 [NM_207678.2], Chkb [NM_007692.6].

In some embodiments, the Localization Domain can bind to polymerase II and is derived or isolated from an aptamer or long noncoding RNA.

In some embodiments, the Localization Domain is derived or isolated from a short interspersed element (SINE). In some embodiments, the SINE is derived or isolated from a gene selected from the group consisting of ENSMUST00000064097, ENSMUST00000066988, ENSMUST00000074862, ENSMUST00000093950, ENSMUST00000095448, ENSMUST00000099693, ENSMUST00000105109, ENSMUST00000108741, ENSMUST00000109431, ENSMUST00000123368. ENSMUST00000124068, ENSMUST00000124095, ENSMUST00000124363, ENSMUST00000124434, ENSMUST00000124813, ENSMUST00000124848, ENSMUST00000125374, ENSMUST00000126063, ENSMUST00000126467, ENSMUST00000127001, ENSMUST00000127328, ENSMUST00000128305, ENSMUST00000129082, ENSMUST00000129910, ENSMUST00000130092, ENSMUST00000130362, ENSMUST00000130582, ENSMUST00000130679, ENSMUST00000131042, ENSMUST00000132070, ENSMUST00000132337, ENSMUST00000132370, ENSMUST00000132414, ENSMUST00000133960, ENSMUST00000134264, ENSMUST00000134795, ENSMUST00000134921, ENSMUST00000135423, ENSMUST00000135564, ENSMUST00000135987, ENSMUST00000136555, ENSMUST00000136749, ENSMUST00000137629, ENSMUST00000137706, ENSMUST001000137776, ENSMUST0000138291, ENSMUST00000138295, ENSMUST00000138574, ENSMUST00000139190, ENSMUST00000139424, ENSMUST00000139529, ENSMUST00000139576, ENSMUST00000139973, ENSMUST00000140009, ENSMUST00000140203, ENSMUST00000140298, ENSMUST00000141088, ENSMUST00000141452, ENSMUST00000141869, ENSMUST00000142279, ENSMUST00000142569, ENSMUST00000142581, ENSMUST00000143133, ENSMUST00000143260, ENSMUST00000143346, ENSMUST00000143649, ENSMUST00000143964, ENSMUST00000144006, ENSMUST00000144043, ENSMUST00000144368. ENSMUST00000144607, ENSMUST00000145549, ENSMUST001000146043, ENSMUST0000146372, ENSMUST00000146404, ENSMUST00000146531, ENSMUST00000146587, ENSMUST00000146644, ENSMUST00000146690, ENSMUST00000146963, ENSMUST00000147541, ENSMUST00000147722, ENSMUST00000148405, ENSMUST00000148534, ENSMUST00000148548, ENSMUST00000149025, ENSMUST00000149382, ENSMUST00000149481, ENSMUST00000149618, ENSMUST00000149815, ENSMUST00000150171, ENSMUST00000150265, ENSMUST00000150455, ENSMUST00000150482, ENSMUST00000150628, ENSMUST00000151038, ENSMUST00000151599, ENSMUST00000151979, ENSMUST00000152025, ENSMUST00000152172, ENSMUST00000152439, ENSMUST00000153815, ENSMUST00000152825, ENSMUST00000152987, ENSMUST0000153589, ENSMUST00000153817, ENSMUST00000154085, ENSMUST00000155540, ENSMUST00000155758, ENSMUST00000156149, ENSMUST00000156150, ENSMUST00000156331, ENSMUST00000156350, ENSMUST00000156633, ENSMUST00000162565, ENSMUST00000163052, ENSMUST00000163302, ENSMUST00000164379, ENSMUST00000165505, ENSMUST00000170933, ENSMUST00000172285, ENSMUST00000172817, ENSMUST00000172838, ENSMUST00000174057, ENSMUST00000174630, ENSMUST00000174768, ENSMUST00000176201, ENSMUST00000176366, ENSMUST00000177104, ENSMUST00000177482, ENSMUST00000178424, ENSMUST00000178920, ENSMUST00000179324, ENSMUST00000180379, ENSMUST00000180382, ENSMUST00000180383, ENSMUST00000180404, ENSMUST00000180410, ENSMUST00000180411, ENSMUST00000180426, ENSMUST00000180434, ENSMUST00000180445, ENSMUST00000180452, ENSMUST00000180466, ENSMUST00000180467, ENSMUST00000180468, ENSMUST00000180477, ENSMUST00000180485, ENSMUST0000018(1495, ENSMUST00000180505, ENSMUST00000180506, ENSMUST00000180509, ENSMUST00000180512, ENSMUST00000180518, ENSMUST00000180527, ENSMUST00000180529, ENSMUST00000180534, ENSMUST00000180538, ENSMUST00000180558, ENSMUST00000180562, ENSMUST00000180576, ENSMUST00000180586, ENSMUST00000180590, ENSMUST00000180595, ENSMUST00000180598, ENSMUST00000180599, ENSMUST00000180601, ENSMUST00000180609, ENSMUST100000180613, ENSMUST00000180623, ENSMUST00000180650, ENSMUST00000180653, ENSMUST00000180670, ENSMUST00000180671, ENSMUST00000180679, ENSMUST00000180682, ENSMUST00000180685, ENSMUST00000180691, ENSMUST00000180693, ENSMUST00000180712, ENSMUST00000180732, ENSMUST00000180733, ENSMUST00000180738, ENSMUST00000180741, ENSMUST00000180748, ENSMUST00000180750, ENSMUST00000180751, ENSMUST00000180779, ENSMUST00000180783, ENSMUST00000180785, ENSMUST00000180797, ENSMUST00000180800, ENSMUST00000180807, ENSMUST00000180809, ENSMUST00000180812, ENSMUST00000180815, ENSMUST00000180832, ENSMUST00000180834, ENSMUST00000180841, ENSMUST00000180842, ENSMUST00000180855, ENSMUST00000180860, ENSMUST00000180864, ENSMUST00000180865, ENSMUST00000180875, ENSMUST00000180876, ENSMUST00000180882, ENSMUST00000180892, ENSMUST00000180896, ENSMUST00000180908, ENSMUST00000180917, ENSMUST00000180920, ENSMUST00000180927, ENSMUST00000180936, ENSMUST00000180942, ENSMUST00000180969, ENSMUST00000180970, ENSMUST00000180975, ENSMUST00000180981, ENSMUST00000181000, ENSMUST00000181003, ENSMUST00000181005, ENSMUST00000181020, ENSMUST00000181022, ENSMUST00000181029, ENSMUST00000181030, ENSMUST00000181041, ENSMUST00000181052, ENSMUST00000181056, ENSMUST00000181066, ENSMUST00000181073, ENSMUST00000181083, ENSMUST00000181085, ENSMUST00000181090, ENSMUST00100181097, ENSMUST00000181106, ENSMUST00000181113, ENSMUST00000181119, ENSMUST00000181124, ENSMUST00000181125, ENSMUST00000181133, ENSMUST00000181140, ENSMUST00000181144, ENSMUST00000181148, ENSMUST00000181149, ENSMUST00000181152, ENSMUST00000181153, ENSMUST00000181160, ENSMUST00000181167, ENSMUST0000181175, ENSMUST00000181180, ENSMUST00000181191, ENSMUST00000181193, ENSMUST00000181197, ENSMUST00000181200, ENSMUST00000181203, ENSMUST00000181206, ENSMUST00000181207, ENSMUST00000181220, ENSMUST00000181230, ENSMUST00000181255, ENSMUST00000181262, ENSMUST00000181265, ENSMUST00000181270, ENSMUST00000181274, ENSMUST00000181301, ENSMUST00000181302, ENSMUST00000181303, ENSMUST00000181304, ENSMUST00000181305, ENSMUST00000181307, ENSMUST00000181311, ENSMUST00000181315, ENSMUST00000181317, ENSMUST00000181328, ENSMUST00000181371, ENSMUST00000181382, ENSMUST00000181395. ENSMUST00000181400, ENSMUST00000181405, ENSMUST00000181416, ENSMUST0000181418, ENSMUST00000181425, ENSMUST0000181426, ENSMUST00000181440, ENSMUST00000181453, ENSMUST00000181454, ENSMUST00000181457, ENSMUST00000181458, ENSMUST00000181460, ENSMUST00000181462, ENSMUST00000181481, ENSMUST00000181482, ENSMUST00000181486, ENSMUST00000181491, ENSMUST00000181498, ENSMUST00000181499, ENSMUST00000181500, ENSMUST00000181503, ENSMUST00000181506, ENSMUST00000181522, ENSMUST00000181526, ENSMUST00000181531, ENSMUST00000181534, ENSMUST00000181538, ENSMUST00000181539. ENSMUST00000181540, ENSMUST00000181546, ENSMUST00000181552, ENSMUST00000181555, ENSMUST00000181556, ENSMUST00000181561, ENSMUST00000181570, ENSMUST00000181574, ENSMUST00000181576, ENSMUST00000181578, ENSMUST100000181587, ENSMUST00000181612, ENSMUST00000181617, ENSMUST00000181631, ENSMUST00000181637, ENSMUST00000181664, ENSMUST00000181668, ENSMUST00000181680, ENSMUST00000181682, ENSMUST00000181687, ENSMUST00000181706, ENSMUST00000181713, ENSMUST00000181717, ENSMUST00000181719, ENSMUST00000181720, ENSMUST00000181723. ENSMUST00000181727, ENSMUST00000181729, ENSMUST00000181732, ENSMUST00000181746, ENSMUST00000181765, ENSMUST00000181769. ENSMUST00000181771, ENSMUST00000181801, ENSMUST00000181803, ENSMUST00000181805, ENSMUST00000181807, ENSMUST00000181811, ENSMUST00000181831, ENSMUST00000181842, ENSMUST00000181846, ENSMUST00000181858, ENSMUST00000181866, ENSMUST00000181872, ENSMUST00000181875, ENSMUST00000181885, ENSMUST00000181890, ENSMUST00000181891, ENSMUST00000181915, ENSMUST00000181918, ENSMUST00000181920, ENSMUST00000181925, ENSMUST00000181928, ENSMUST00000181942, ENSMUST00000181973.

In some embodiments, the Localization Domain can bind to proteins involved in transcription. In some embodiments, the Localization Domain can bind to proteins involved in RNA splicing.

In some embodiments, the Localization Domain can promote accumulation of the trans-splicing molecule in nuclear paraspeckles. In some embodiments, the Localization Domain that promotes accumulation of the trans-splicing molecule in nuclear paraspeckles can be derived or isolated from a gene selected from the group consisting of: lnc-LTBP3-10 [lnc-LTBP3-10], SLC29A2 [ENSG00000174069.12], SNHG1 [ENSG00000255717.7], MUS81 [ENSG00000172732.12], TCIRG1 [ENSG00000110719.10], INPPL1 [ENSG00000165458.14], lnc-ANAPC11-7 [lnc-ANAPC11-7], IL18BP [ENSG00000137496.18], POLA2 [ENSG00000014138.9], PCNX3 [ENSG00000197136.4], PC [ENSG00000173599.15], RBM4 [ENSG00000173933.20], lnc-KCNK7-6 [lnc-KCNK7-6], EML3 [ENSG00000149499.11], PGGHG [ENSG00000142102.16], RBM14 [ENSG00000239306.4], LTBP3 [ENSG00000168056.16], ATG2A [ENSG00000110046.13], XLOC_026224 [XLOC_026224], HERC2P2 [ENSG00000276550.4], WDR90 [ENSG00000161996.19], lnc-LTBP3-2 [lnc-LTBP3-2], LENG8 [ENSG00000167615.16], TPCN2 [ENSG00000162341.18], lnc-TCIRG1-1 [lnc-TCIRG1-1], ATG16L2 [ENSG00000168010.11], MROH1 [ENSG00000179832.17], CCDC57 [ENSG00000176155.19], lnc-LTBP3-11 [lnc-LTBP3-11], PIDD1 [ENSG00000177595.18], lnc-VSTM5-1 [hnc-VSTM5-1], NEAT1 [ENSG00000245532.9], XLOC_079850 [XLOC_079850], XLOC_028656 [XLOC_028656], DNHD1 [ENSG00000179532.12], ABCA7 [ENSG0000064687.12], XLOC_000636 [XLOC_000636], MAN2C1 [ENSG00000140400.17], lnc-SSH3-5 [lnc-SSH3-5], MIRLFT7BHG [ENSG00000197182.14], MAMDC4 [ENSG00000177943.14], NAA40 [ENSG00000110583.13], ANKRD13D [ENSG00000172932.14], lnc-NUMA1-3 [lnc-NUMA1-3], ADAMTS10 [ENSG00000142303.14], XLOC_083799 [XLOC_083799], ARHGEF17 [ENSG00000110237.5], CDC42BPG [ENSG00000171219.9], SNAPC4 [ENSG00000165684.4], lnc-CFL1-1 [lnc-CFL1-1], B4GALNT4 [ENSG00000182272.12], XLOC_027567 [XLOC_027567], XLOC_000644 [XLOC_000644], XLOC_024022 [XLOC_024022], LTO1 [ENSG00000149716.12], AC064843.1 [ENSG00000286621.1], CHRND [ENSG00000135902.10], ASPSCR1 [ENSG00000169696.16], RAD9A [ENSG00000172613.8], lnc-RTN4R-1 [lnc-RTN4R-1], lnc-MRPL 11-1 [lnc-MRPL 11-1], SSI-13 [ENSG00000172830.13], XLOC_000637 [XLOC_000637], AP000873.2 [ENSG00000247137.9], lnc-TRPT1-4 [lnc-TRPT1-4], XLOC_027568 [XLOC_027568], LINC01503 [ENSG00000233901.6], RNASEH2C [ENSG00000172922.9], XLOC_000634 [XLOC_000634], MYO7A [ENSG00000137474.22], XLOC_000633 [XLOC_000633], lnc-BCL3-1 [lnc-BCL3-1], MTMR9LP [ENSG00000220785.7], AT5B1 [ENSG00000254470.3], lnc-EDT1-2 [lnc-EDF1-2], lnc-UNC93B1-1 [lnc-UNC93B1-1], GOLGA8B [ENSG000000215252.11], MSH5 [ENSG00000204410.15], AP003119.1 [ENSG00000254632.2], GUSBP11 [ENSG00000228315.12], RPS6KB2 [ENSG00000175634.15], EME2 [ENSG00000197774.13], XLOC_028057 [XLOC_028057], FRMD8 [ENSG00000126391.14], lnc-OGFOD3-1 [lnc-OGFOD3-1], XLOC_152482 [XLOC_152482], XLOC_028434 [XLOC_028434], ZNF276 [ENSG00000158805.12], AP000944.5 [ENSG0000285816.1], NRBP2 [ENSG00000185189.18], NDOR1 [ENSG00000188566.13], lnc-PHYHD1-1 [lnc-PHYHD1-1], lnc-RECQL4-3 [lnc-RECQL4-3], lnc-UAP1L1-4 [lnc-UAP1L1-4], MSH5-SAPCD1 [ENSG00000255152.8], lnc-P2RY6-1 [lnc-P2RY6-1], RELT [ENSG00000054967.13], CPNE7 [ENSG00000178773.15], XLOC_028557 [XLOC_028557], XLOC_156663 [XLOC_156663], CORO6 [ENSG00000167549.18], RTEL1 [ENSG00000258366.8], MIR34AHG [ENSG00000228526.7], STPG3-AS1 [ENSG00000275549.1], lnc-WFIKKN2-4 [lnc-WFIKKN2-4], SYNGAP1 [ENSG00000197283.17], LRRC45 [ENSG00000169683.8], KIAA089SL [ENSG00000196123.13], PNKP [ENSG00000039650.12], lnc-EIF1AD-5 [lnc-EIF1AD-5], TM7SF2 [ENSG00000149809.15], NSUNSP2 [ENSG00000106133.18], lnc-POLR2L-1 [lnc-POLR2L-1], lnc-PPP1R27-1 [lnc-PPP1R27-1], ACC 10285.2 [ENSG00000262877.5], lnc-LRRC; 32-5 [lnc-LRRC32-5], AC131009.4 [EN5000000279283.1], BBS1 [ENSG00000174483.20], XLOC_061408 [XLOC_061408], lnc-SERPINH1-3 [lnc-SERPINH1-3], AC027601.6 [ENSG00000287431.1], lnc-NFAM1-3 [lnc-NFAM1-3], EXD3 [ENSG00000187609.16], Ac009022.1 [ENSG00000196696.12], MC1R [ENSG00000258839.3], PKD1P6 [ENSG00000250251.6], lnc-KLHL35-6 [lnc-KLHL35-6], Z97832.2 [ENSG00000272374.11], C19orf25 [ENSG00000119559.16], lnc-TMEM 138-3 [lnc-TMEM138-3], AL031595.3 [ENSG00000280434.1], lnc-LRRC56-3 [lnc-LRRCS6-3], lnc-STIP1-2 [lnc-STIP1-2], XLOC_09.5699 [XLOC_095699], SSSCA1-AS1 [ENSG00000260233.3], NPDC1 [ENSG00000107281.10], lnc-NR1D1-1 [lnc-NR1D1-1], lnc-RPL12-1 [lnc-RPL12-1], lnc-MRPL49-1 [lnc-MRPL49-1], XLOC_061398 [XLOC_061398], TOB1-AS1 [ENSG00000229980.5], AC127502.1 [ENSG00000215302.8], XLOC_149046 [XLOC_149046], lnc-TRMT12-4 [lnc-1RMT112-4], LINC:02593 [ENSG00000223764.2], KLHL17 [ENSG00000187961.14], lnc-KLHL35-7 [lnc-KLHL35-7], lnc-TMEM258-2 [lnc-TMEM258-2], AP002495.1 [ENSG00000254469.7], XLOC_024025 [XLOC_024025], GPSM1 [ENSG00000160360.13], XLOC_152839 [XLOC_152839], LBHD1 [ENSG00000162194.12], GATD1 [ENSG00000177225.17], XLOC_149045 [XLOC_149045], LENG8-AS1 [ENSG00000226696.6], MAP4K2 [ENSG00000168067.12], C11orf80 [ENSG00000173715.16], MAPK8IP3 [ENSG00000138834.12], XLOC_090526 [XLOC_090526], KIFC2 [ENSG00000167702.12], LRPSL [ENSG00000100068.13], SEC31B [ENSG00000075826.17], XLOC_024171 [XLOC_024171], PPP2RSB [ENSG00000068971.14], lnc-G1PC3-3 [lnc-G1PC3-3], AC020916.1 [ENSG00000267519.6], XLOC_156901 [XLOC_156901], AP006333.1 [ENSG00000256341.1], lnc-ZNF778-3 [lnc-ZNF778-3], lnc-LAMA5-1 [lnc-LAMA5-1], lnc-TMEM106A-3 [lnc-TMEM106A-3], lnc-ACER3-1 [lnc-ACER3-1], RHPN1 [ENSG00000158106.14], XLOC_028558 [XLOC_028558], XLOC_088401 [XLOC_088401], BX255925.3 [ENSG00000284976.1], GUCY2EP [ENSG00000204529.4], XLOC_152506 [XLOC_152506], NOXA 1 [EN5000000188747.8], lnc-ARRDC1-2 [lnc-ARRDC1-2], XLOC_145191 [XLOC_145191], BSCL2 [ENSG00000168000.14], lnc-MACROD1-1 [lnc-MACROD1-1], AL162586.1 [ENSG00000225032.5], AP000944.7 [ENSG00000287917.1], AC091196.1 [ENSG00000285581.1], ZNRD2 [ENSG00000173465.8], XLOC_026268 [XLOC_026268], OSBPL7 [ENSG00000006025.12], lnc-SSH3-4 [lnc-SSH3-4], C9orf106 [ENSG00000179082.3], AP000437.1 [ENSG00000279549.1], lnc-NCOA3-14 [lnc-NCOA3-14], NADSYN1 [ENSG100000172890.13], XLOC_060204 [XLOC_060204], lnc-SHANK2-1 [lnc-SHANK2-1], MEGF6 [ENSG00000162591.16], AC099811.1 [ENSG00000236194.3], ME3 [ENSG000000151376.16], XLOC_028655 [XLOC_028655], GDPD5 [ENSG00000158555.15], lnc-SPDYC-2 [lnc-SPDYC-2], AC008105.3 [ENSG00000267121.6], lnc-NCOA3-21 [lnc-NCOA3-21], lnc-FEN1-6 [lnc-FEN1-6], lnc-HYOU1-1 [lnc-HYOU1-1], AC102953.2 [ENSG00000273230.1], XLOC_095073 [XLOC_095073], LINC00235 [ENSG00000277142.1], AL355987.4 [ENSG00000273066.5], XLOC_152404 [XLOC_152404], lnc-CDK12-1 [lnc-CDK12-1], XLOC_028004 [XLOC_028004], lnc-CCDC154-2 [lnc-CCDC154-2], lnc-CCDC87-1 [lnc-CCDC87-1], INPP5E [ENSG00000148384.13], XLOC_021222 [XLOC_021222], AJM1 [ENSG00000232434.2], HSF4 [ENSG00000102878.16], LIN00313 [ENSG00000185186.10], lnc-UNC93131-7 [lnc-UNC93131-7], lnc-PIDD1-2 [lnc-PIDD1-2], lnc-CSNK1G2-5 [lnc-CSNK1G2-5], lnc-UNC93B1-5 [lnc-UNC93B1-5], AP006621.3 [ENSG00000255284.2], CCDC78 [ENSG00000162004.17], lnc-UUAA0-7 [lnc-1HAAO-7], EFEMP2 [ENSG00000172638.13], XLOC_000635 [XLOC_00063S], XLOC_147952 [XLOC_00147952], lnc-PKNOX1-1 [lnc-PKNOX1-1], lnc-LTBP3-9 [lnc-LTP3-9], AC008895.1 [ENSG00000279948.1], lnc-TBCID3H-7 [lnc-TBC1D31-1-7], lnc-TMEM250-3 [lnc-TMEM250-3], lnc-CDC42EP2-1 [lnc-CDC42EP2-1], AC08774 1.1 [ENSG00000262580.5], XLOC_156972 [XLOC_156972], lnc-PC-3 [lnc-PC-3], AC0090589.3 [ENSG0000271060.1], XLOC_045084 [XLOC_045084], TIAF1 [ENSG00000221995.5], lnc-CYBA-4 [lnc-CYBA-4], lnc-SLC11A2-7 [lnc-SLC11A2-7], AC0141586.1 [ENSG00000215154.6], AP003559.1 [ENSG00000256443.1], XLOC_095076 [XLOC_095076], PNPLA7 [ENSG00000130653.16], lnc-RNF 166-5 [lnc-RNF166-5], XLOC_023911 [XLOC_023911], AC092127.1 [ENSG00000260417.1], lnc-TRPT1-3 [lnc- TRPT1-3], XLOC_028195 [XLOC_028195], XLOC_080106 [XLOC_080106], XLOC_026739 [XLOC_026739], lnc-NUP98-1 [lnc-NUP98-1], HDAC10 [ENSG00000100429.18], DRD4 [ENSG00000069696.7], lnc-DOC2B-3 [lnc-DOC2B-3], lnc-DOLK-1 [lnc-DOLK-1], CNIH2 [ENSG00000174871.11], RGL3 [ENSG00000205517.12], GALT [ENSG00000213930.11], AP001107.9 [ENSG00000255468.7], lnc-MKNK2-1 [lnc-MKNK2-1], AL033543.1 [ENSG00000279175.1].

In some embodiments, the Localization Domain can promote accumulation of the trans-splicing molecule to nuclear speckles. In some embodiments, the Localization Domain that promotes accumulation of the trans-splicing molecule to nuclear speckles can be derived or isolated from a gene selected from the group consisting of: MALAT1 [NR_002819.4], MEG3[ENSG00000214548], XLOC_003526 [ENSG00000250657], In some embodiments, the Localization Domain promotes accumulation of the trans-splicing molecule to nuclear speckles via binding to a protein selected from the group consisting of: SRSF1 [ENSG00000136450], SRSF2 [ENSG00000161547], SRSF3 [ENSG00000112081], SRSF4 [ENSG00000116350], SFSF6 [ENSG00000124193], SFSF7 [ENSG00000115875], SRSF10 [ENSG00000188529], SRSF11 [ENSG00000116754], CLK1 [ENSG00000013441], CLK2 [ENSG00000176444].

In some embodiments, the Localization Domain can promote accumulation of the trans-splicing molecule in nuclear speckles via association to a protein. In some embodiments, this protein is selected from group consisting of: ADNP [ENSG00000101126], ANXA7 [ENSG00000138279], API5 [ENSG00000166181], AQR [ENSG00000021776], ATAD2 [ENSG00000156802], BAZ1B [ENSG00000009954], BCLAF1 [ENSG00000029363], BTAF1 [ENSG00000095564], CCAR1 [ENSG00000060339], CCAR2 [ENSG00000158941], CDC5L [ENSG00000096401], CDC73 [ENSG00000134371], CDK11B [ENSG00000248333], CDK12 [ENSG00000167258], CDKN2AIP [ENSG00000168564], CHD3 [ENSG00000170004], CHD4 [ENSG00000111642], CHTF18 [ENSG00000127586], CPSF1 [ENSG00000071894], CSTF3 [ENSG00000176102], CTCR9 [ENSG00000198730], CUL3 [ENSG00000036257], CUL4B [ENSG00000158290], CWC22 [ENSG00000163510], CWF19L1 [ENSG00000095485], DDX23 [ENSG00000174243], DHX16 [ENSG00000123136], DDX42 [ENSG00000198231], DDX46 [ENSG00000145833], DHX16 [ENSG00000204560], DHX38 [ENSG00000140829], DNMT1 [ENSG00000130816], ELOA [ENSG00000011007], EWSR1 [ENSG00000182944], FAF1 [ENSG000001851014], FBXO22 [ENSG00000167196], FKBP5 [ENSG00000096060], FUBP1 [ENSG00000162613], FUBP3 [ENSG0000010764], OPATCH8 [ENSG00000186566], GPS1 [ENSG00000169727], GTF3C1 [ENSG100000077235], GTF3C4 [ENSG00000125484], GTF3C5 [ENSG00000148308], HCFC1 [ENSG00000172534], HELLS [ENSG00000119969], IK [ENSG00000113141], ILF2 [ENSG00000143621], INTS13 [ENSG00000064102], KDM1A [ENSG00000004487], KHDRBS1 [ENSG00000121774], KHSRP [ENSG00000088247], LIG1 [ENSG00000105486], MATR3 [ENSG00000280987], METTL1 [ENSG00000037897], MRE11 [ENSG00000020922], MSH2 [ENSG00000095002], MSH3 [ENSG00000113318], MS116 [ENSG00000116062], NBN [ENSG00000104320], NCBP1 [ENSG00000136937], NONO [ENSG00000147140], PAF1 [ENSG00000006712], PDS5B [ENSG00000083642], POLD1 [ENSG00000062822], POLR2A [ENSG00000181222], POLR2B [ENSG00000047315], PPM1G [ENSG0000115241], PP1R10 [ENSG00000204569], PRPF19 [ENSG00000110107], PRPF3 [ENSG00000117360], PRPF31 [ENSG00000105618], PRPF40A [ENSG00000196504], PRPF4B [ENSG00000112739], PRPF6 [ENSG00000101161], PSPC1 [ENSG00000121390], PTBP2 [ENSG0000117569], PUS7 [ENSG00000091127], RAD21 [ENSG00000164754], RAD50 [ENSG00000113522], RALY [ENSG00000125970], RBM10 [ENSG00000182872], RBM12 [ENSG00000244462], RBM14 [ENSG00000239306], RBM17 [ENSG00000134453], RBM25 [ENSG00000119707], RBM26 [ENSG00000139746], RBM4 [ENSG00000173933], RBMX [ENSG00000147274], RFC1 [ENSG00000035928], RFC4 [ENSG00000163918], RNF20 [ENSG00000155827], RNF40 [EN5000000103549], RNMT [ENSG00000101654], RPL35A [ENSG00000182899], RPRD1B [ENSG00000101413], RPRD2 [ENSG00000163125], SAMHD1 [ENSG00000101347], SART1 [ENSG00000175467], SART3 [ENSG00000075856], SBNO1 [ENSG00000139697], SF3A1 [ENSG00000099995], SF3B1 [ENSG00000115524], SF3B2 [ENSG00000087365], SFPQ [ENSG00000116560], SIN3A [ENSG00000169375], SLC4A1AP [ENSG00000163798], SMARCC1 [ENSG00000173473], SMU1 [ENSG00000122692], SON [ENSG00000159140], STAG2 [ENSG00000101972], SUGT1 [ENSG00000165416], SUPT5H [ENSG00000196235], SUPT6H [ENSG00000109111], SYMPK [ENSG00000125755], TARDBP [ENSG00000120948], TCERG1 [ENSG00000113649], THOC2 [ENSG00000125676], THOCS [ENSG00000100296], TP53BP1 [ENSG00000067369], TRMT1 [ENSG00000104907], TRMT1L [ENSG00000121486], TSR1 [ENSG00000167721], UBR5 [ENSG00000104517], UHHRF1 [ENSG00000276043], USP39 [ENSG00000168883], USP48 [ENSG00000090686], USP7 [ENSG00000187555], WAG [ENSG00000095787], WDHD1 [ENSG00000198554], WRNIP1 [ENSG00000124535], XPO5 [ENSG00000124571], XPO7 [ENSG00000130227], XPOT [ENSG00000184575], YLPM 1 [ENSG00000119596], ZC3H11A [ENSG00000058673], ZC31H114 [ENSG00000100722], ZMYND8 [ENSG00000101040], ZNF326 [ENSG00000162664].

In some embodiments, the Localization Domain sequence(s) can be isolated or derived from a long non-coding RNA that is involved in transcriptional regulation. In some embodiments, the long non-coding RNA comprises Air. Alpha 250/Alpha 280, ANRIL, Beta-globin transcripts, Beta-MHC antisense transcripts, CAR Intergenic 10, CCND1 associated ncRNAs, COLDAIR, COOLAIR, DHFR upstream transcripts, Emx2os, Evf2, fbp1+ promoter RNAs, GAL10-ncRNA, H19, H19 antisense, H19 upstream conserved 1 and 2, H19 ICR ncRNAs, HOTAIRM1, HOTTIP, Hoxa11as, ICR1, Kcnglot1, Khps1a, L1PA16, LINoCRb, MEG3, Mistral, Msx1as, Ncspas, ncR-Upar, PHO5 lncRNA, P1-1084 antisense, pRNA, PWR1, RTL, SRG1, TEA ncRNAs, TiR1axut, TPO1axut, Tsix, Xist, 7SK, B2 SINE RNA, GAS5, HOTAIR, Jpx, LXRBSV, PR antisense transcripts, VL30 RNAs, Adapt33, antiPeg11, Glt2-as, HOXA3as, HOXA6as, linc1242, linc1257, linc1368, linc1547, linc1582, linc1609, linc1610, lincRNA-p21, lincRNA-RoR b, Malat1-as, MEG9, NDM29, NEAT1, PANDA, PLAT-1, Rian, SatIII transcripts, SNHG3, SRA, Tmcvpg1, TncRNA, TUG1, or another combination thereof.

In some embodiments, the Localization Domain sequence(s) can be isolated or derived from a long non-coding RNA that is involved in splicing regulation. In some embodiments, the long non-coding RNA comprises M1AT, LUST, Malat1, SAN, VL30 RNAs Zeb2NAT, or any combination thereof.

In some embodiments, the Localization Domain sequence(s) can be directly adjacent to an Antisense Domain. In some embodiments, the Localization Domain sequence(s) can be directly adjacent to the Replacement Domain.

In some embodiments, the Localization Domain(s) can be adjacent to a 5′ end of a trans-splicing molecule. In some embodiments, the Localization Domain(s) are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotide, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotide, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotide, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 400 nucleotides, 500 nucleotides, more than 500 nucleotides, or any number of nucleotides in between distant from the 5′ end of the trans-splicing molecule.

In some embodiments, the Localization Domain(s) can be adjacent to the 3′ end of the trans-splicing molecule. In some embodiments, the Localization Domain(s) are 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotide, 19 nucleotides, 20 nucleotides, 21 nucleotides, 22 nucleotides, 23 nucleotide, 24 nucleotide, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotide, 100 nucleotide, 110 nucleotides, 120 nucleotides, 130 nucleotides, 140 nucleotides, 150 nucleotides, 160 nucleotides, 170 nucleotides, 180 nucleotide, 190 nucleotides, 200 nucleotides, 250 nucleotides, 300 nucleotides, 400 nucleotides, 500 nucleotide, more than 500 nucleotides, or any number of nucleotides in between distant from the 3′ end of the trans-splicing molecule.

In some embodiments, the Localization Domain(s) can be 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotide, 19 nucleotides, 20 nucleotides, 21 nucleotides, 22 nucleotides, 23 nucleotide, 24 nucleotide, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, 150 nucleotides, 160 nucleotides, 170 nucleotides, 180 nucleotide, 190 nucleotides, 200 nucleotides, 250 nucleotides, 300 nucleotides, 400 nucleotides, 500 nucleotides, more than 500 nucleotides, or any number of nucleotides in between distant from the first nucleotide of the Replacement Domain or Antisense Domain in the 5′ direction.

In some embodiments, Localization Domain(s) can bel nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotide, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 31 nucleotides, 32 nucleotides, 33 nucleotides, 34 nucleotides, 35 nucleotides, 36 nucleotides, 37 nucleotides, 38 nucleotide, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotide, 120 nucleotides, 130 nucleotides, 140 nucleotides, 150 nucleotides, 160 nucleotides, 170 nucleotides, 180 nucleotide, 190 nucleotides, 200 nucleotides, 250 nucleotides, 300 nucleotides, 400 nucleotides, 500 nucleotides, more than 500 nucleotides, or any number of nucleotides in between distant from the last nucleotide of the Replacement Domain or Antisense Domain in the 3′ direction.

In some embodiments, the trans-splicing molecule may comprise a Localization Domain. In some embodiments, the trans-splicing molecule may comprise 2 or more Localization Domains. In some embodiments, the trans-splicing molecule comprises 1, 4, 5, 6, 7, 8, 9, 10, 11, 12, 13, 14, 15, 20, 30, 40, 50, 75, 100, 200, 300 or more Localization Domains.

Compositions comprising localization sequences disclosed herein can include any sequences that promote nuclear or subnuclear localization of trans-splicing molecules. Non-limiting examples of localization sequences can include sequences that promote localization of trans-splicing molecules to the cellular nucleus from the cytoplasm or to specific structures within the nucleus such as nuclear speckles or paraspeckles. In some embodiments, the localization sequences can also include sequences that promote association of the trans-splicing molecule with nuclear-localized proteins and protein complexes such as the spliceosome, transcriptional proteins, or splicing factors.

The RNA trans-splicing technology, which involves the inclusion of specific localization sequences for trans-splicing molecules, is among the first to show RNA-trans-splicing with high efficiency against multiple RNA targets. Highly efficient RNA trans-splicing has at least three primary advantages over other RNA trans-splicing systems. First, this improved efficiency can replace defective RNA sequences at levels sufficient to reconstitute the activity of mutated genes to treat recessive genetic disorders. Indeed, treatment of many recessive gene disorders may require at least 30% efficiency, wherein 100% efficiency denotes complete replacement of a sequence within a Target RNA. Second, this improved efficiency can enable compositions as described herein to replace defective target RNA sequences at levels sufficient to treat dominant genetic disorders. For example, as a single mutated allele is sufficient to cause disease, many diseases in this class require highly-efficient replacement of mutated sequences as the mutated sequences may cause toxicity. As a result, even higher efficiency is required, e.g., at least about 70%. Thus, compositions as described herein can more effectively target broader classes of genetic disorders, i.e., even those with single mutated allele. Finally, the broad ability of our RNA trans-splicing technology to modify multiple Target RNAs demonstrates the first broadly-applicable and efficient version of this technology. This is a very general capability, with this disclosure providing demonstrations of RNA trans-splicing system that can efficiently replace sequences with multiple target RNAs.

The inclusion of localization sequences in trans-splicing molecules to form the RNA trans-splicing technology described herein can be a general capability that may further allow the alteration of non-coding sequences within target RNAs. By replacing the 5′ or 3′ untranslated regions of Target RNAs with high efficiency, the methods and composition described herein may allow the alteration of RNA behaviors such as translation or turnover. The net result of these effects can be increased production of protein from Target RNAS or other downstream effects associated with altered RNA levels.

RNA sequences can influence localization of RNAs. The present disclosure provides localization sequences, and analyzes the activity of these known localization sequences in the context of RNA trans-splicing. Further, sequences may be found within RNAS that display nuclear-specific localization patterns. These sequences may influence RNA localization in a heterologous context such as within a trans-splicing RNA. These localization sequences may increase the efficiency of RNA-trans-splicing when placed at the 5′ end, 3′ end, or within of a model trans-splicing molecule. The present disclosure provides sequences that may promote nuclear or subnuclear localization. In some embodiments, the sequences do not influence trans-splicing activity. Ile present disclosure provides sequences that may promote nuclear or subnuclear localization. In some embodiments, the sequences localize trans-splicing molecules to or within the cellular nucleus. In some embodiments, the localizing of the trans-splicing molecules to or within the cellular nucleus results in increased trans-splicing activity. As used herein, these trans-splicing-specific localizing sequences may be termed “nuclear localization sequences,” “localization sequences,” or the like.

Compositions as described herein may modulate the level of protein produced. In addition to replacing specific mutated sequences within a target RNA with non-mutated sequences, another useful operation of compositions as described herein can be increasing the production of a protein encoded by a target RNA. Small molecule drugs that increase translation by promoting stop codon read-through may suffer extensive off-targets. For example, such small molecule drugs may promote read-through on non-target mRNAs. Further, pre-mature stop codons can cause insufficient protein levels. Engineered tRNAs to block pre-mature termination codons may suffer from this same fundamental issue. An RNA trans-splicing system as disclosed herein, by contrast, can replace sequences in any target mRNA with translation-amplifying sequences to increase protein production. Furthermore, compositions as described herein may have greater target specificity to effect therapy to the appropriate target RNA, and thereby may increase production of a protein encoded by the target RNA. Described herein are methods of efficient RNA trans-splicing mediated by localization sequences, to address a long-felt but unmet need of a method, as recognized herein, to promote targeted amplification of protein production from specific mRNAs.

The present disclosure provides compositions comprising a trans-splicing nucleic acid with one or more localization sequences. The localization sequence described herein may increase the efficiency of nucleic acids at replacing sequences in a target RNA. For example, localization sequences can increase the efficiency of RNA-trans-splicing when placed at the 5′ end, 3′ end, or within of a model trans-splicing molecule.

The trans-splicing molecule may comprise, e.g., DNA or RNA. The trans-splicing nucleic acid may be transcribed from a DNA molecule comprising a Localization Domain. In some embodiments, the DNA or RNA trans-splicing molecule can comprise a Replacement Domain. In some embodiments, the Replacement Domain can be transcribed into an RNA sequence, such as an RNA sequence that corresponds to a missing or mutated portion of a target RNA sequence. In some embodiments, the DNA or RNA trans-splicing molecule can comprise an Antisense Domain. In some embodiments, the Antisense Domain of the DNA molecule can be transcribed into an Antisense Domain comprising RNA. In some embodiments, the Antisense Domain comprising RNA is complementary to the target RNA or a portion thereof. In some embodiments, the Antisense Domain can bind to the target RNA. In some embodiments, the antisense RNA can be chosen so that successful trans-splicing causes removal of micro-open reading frames in the target RNA. In some embodiments, the trans-splicing DNA or RNA molecule can comprise an Intronic Domain. The Intronic Domain of the DNA molecule can be transcribed into an Intronic Domain comprising RNA. In some embodiments, the Intronic Domain can promote the trans-splicing reaction between a trans-splicing nucleic acid molecule and the target RNA. In some embodiments, the Intronic Domains can carry binding sites that are preferentially-targeted by RNA-binding proteins with disease-causing mutations. In some embodiments, the trans-splicing DNA or RNA molecule can comprise a Localization Domain. In some embodiments, the trans-splicing DNA or RNA molecule can comprise one or more Localization Domains. In some embodiments, the DNA molecule comprising one or more Localization Domains can encode an RNA molecule comprising the one or more Localization Domains. In some embodiments, the DNA molecule comprising one or more Localization Domains can be transcribed into an RNA molecule comprising the one or more Localization Domains.

Compositions as described herein can treat mutated target RNA, and thereby amplify protein production form the target RNA. For example, Myotonic dystrophy is caused by RNAs that carry repetitive ‘CUG’ tracts that bind the splicing factor MBNL1. Titration of MBNL1 away from its typical targets causes widespread dysfunction of RNA alternative splicing and is responsible for most manifestations of disease in patients. Described herein are methods of increasing MBNL1 protein production with an efficient RNA trans-splicing approach can address this disease via production of sufficient MBNL1 protein to reconstitute its typical activities in alternative splicing regulation.

Described herein is an RNA trans-splicing system carrying various localization sequences such as, a Woodchuck Hepatitis Virus (WHV) post-transcriptional Regulatory Element (WPRE). Also described herein is a reporter that comprises a firefly luciferase coding sequence and the last 2 exons and intervening introit of MBNL1. This assay is qualitative, not fully quantitative, but is useful because it is what end-users in cell biology often use when attempting to answer scientific questions about the presence, absence, or general magnitude of a transcript. Indeed, this reporter is based on the pMIR-GLO luciferase vector that is used to assess the stability and protein production from a model mRNA.

Experiments were conducted with either transiently-transfected reporter and trans-splicing molecule or systems packaged in lentivirus.

Localization Sequences that Promote Localization of Trans-Splicing Nucleic Acids to the Site of Transcription

In some embodiments, a localization sequence may promote localization of the trans-splicing nucleic acid to the site of transcription. In some embodiments, the localization sequence may bind to RNA polymerase II. In some embodiments the localization sequence may be derived or isolated from a long non-coding RNA that binds to RNA polymerase II. In some embodiments, the localization sequence that binds to RNA polymerase II may be derived or isolated from B2 long non-coding RNA. The sequence may be a DNA sequence. The sequence may be an RNA sequence. In some embodiments, the sequences from B2 long non-coding RNA can comprise or consist of GGGCUGGUGAGAUGGCUCAGUGGGUAAGAGCACCCGACUGCUCUUCCGAAGGUCAGGAG UUCAAAUCCCAGCAACCACAUGGUGGCUCACAACCAUCCGUAACGAGAUCUGAUUCCCUC UUCUGGAGUGUCUGAAGACACAGCUACAGUGUACUU (SEQ ID NO: 1). In some embodiments, the localization sequence can comprise at least about 60%, about 65%, about 70%, about 75%, about 80%, about 85%, about 90%, about 95%, about 97.5%, about 98%, about 99%, or about 100%, identity with a sequence encoded by SEQ ID NO: 1. In some embodiments, the localization sequence can comprise a sequence encoded by SEQ ID NO: 1. The localization sequence may be transcribed into an RNA molecule.

In some embodiments, the localization sequence can be an aptamer that binds RNA polymerize II.

Localization Sequences that Promote Localization of Trans-Splicing Nucleic Acids to or within the Nucleus

In some embodiments, a localization sequence can promote localization of the trans-splicing nucleic acid to or within the nucleus. In some embodiments, a localization sequence may promote localization of the trans-splicing nucleic acids to nuclear speckles. In some embodiments, the localization sequence may be derived from a long non-coding RNA. The sequence may be a DNA sequence. The sequence may be an RNA sequence. In some embodiments, the localization sequence that promotes localization of the trans-splicing molecule to nuclear speckles can comprise or consist of sequences from MALAT1 long non-coding RNA. In some embodiments, the sequences from MALAT1 can comprise or consist of: GGAAUUUGGAGGGAUGGGAGGAGGGGGUGGGGCUUACUUGUUGUAGCUUUUUUUUUUUU UACAGACUUCACAGAGAAUGCAGUUGUCUUGACUUCAGGUCUGUCUGUUCUGUUGGCAA GUAAAUCUCACUACUGUUCUGAUCCCGCUGCUAUUAGAAUCGCAUUGUGAAACGACUGGAG UAUGAUUAAAAGUUGUGUUCCCCAAUGCUUGGAGUAGUGAUUGUUGAAGGAAAAAAUCC AGCUGAGUGAUAAAGGCUGAGGUUGAGGAAAUUUCUGCAGUUUUAAGCAGUCGUAUUU GUGAUUGAAGCUGAGUACAUUUUGCUGGUGUAUUUUUACGGUAAAAUGCUUUUUGUUCAU UUCUGGUGGUGGGAGGGGACUGAAGCCUUUAGUCUUUUCCAGAUGCAACCUUAAAAUCA GUGACAAGAAACAUUCCAAACAAGCAACAGUCUUCAAGAAAUUAAACUGGCAAGUGCIAA AUGUUUAAACAGUUCAGUGAUCUUUAGUGCAUUGUUUAUGUGUGGGUUUCUCUCUCCCC UCCCUUCGGUCUUAAUUCUUACAUGCAGGAACACUCACGCAGACACACGUAUGCGAAGGGCC AGAGAAGCAGACCCAGUAAGAAAAAAUAGCCUAUUUACUUUAAAUAAACCAAACAUUC CAUUUUAAAUGUGGGGAUUGGGAACCACUAGUUCUUUCAGAUGGAUUCUUCAGACUAU ACGAAGGAGCUUCCAGUUGAAUUCACCAGUCGGACAAAAUCGAGGAAAACAGGUGAACAAGC UUUUUCUGUAUUUACAUACAAAGUCAGAUCAGUUAUGGGACAAUAGUAUUGAAUAGAUU UCAGCUUUAUGCUGGAGUAACUGGCAUGUGAGCAAACUGUGUUGGCGUGGGGGUGGAGG GGUGAGGUGGGCGCUAAGCCUUUUUUUAAGAUUUUUCAGGUACCCCUCACUAAAGGCAC CGAAGGCUUAAAGUAGGACAACCAUGGACGCCUUCCUGUGGCAGGAGAGACAACAAAG (SEQ ID NO: 2). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 2. In some embodiments, the localization sequence can comprise a sequence encoded by SEQ ID NO: 2. The localization sequence may be transcribed into an RNA molecule.

In some embodiments, the sequences (e.g., DNA or RNA sequences) from MALAT1 can comprise or consist of: GAGUUAGAAGAAUUUGGAAGGCCUUAAAUAUAGUAGCUUAGUUUGAAAAAUGUGAACGGA CUUUCGUAACGGAAGUAAUUCAAGAUCAAGAGUAAUUACCAACUUAAUGUUUUUGCAUU GGACUUUGAGUUAAGAUUAUUUUUUAAAUCCUGAGCIACUAGCAUUAAUUGACAGCUGAC CCAGGUGCUACACAGAAGUGGAUUCAGUGAAUCUAGGAAGACAGCAGCAGACAGGGAUUC CAGGAACCAGUGUUUGAUGAAGCUAGGACUGAGGAGCAAGCGAGCAAGCAGCAGUUCGU GGUGAAGAUAGGAAAAGAGUCCAGGAGCCAGUGCGAUUUGGUGAAGGAAGCUAGGAAGA AGGAAGGAGCGCUAACGAUUUGGUGGUGAAGCUAGGAAAAAGGAUUCCAGGAAGGAGCG AGUCGCAAUUUGGUGAUGAAGGUAGCAGGCGGCUUGGCUUGGCAACCACACGGAGGAGGC GAGCAGGCGUUGUGCGUAGACGGAUCCUAGACCAGCAUGCCAGUGUGCCAAGGCCACAGGG AAAGCGAGUGGUUGGUAAAAAUCCGUGAGGUCGGGAAUAUGUUGUUUUUCUGGAACUUA CUUAUGGUAAGCUUUUAUUUAUUUUCUAAUAUAAUGGGGGGAGUUUCGUACUGAGGUGUA AAGOGAUUUAUAUGGGGACGUAGGCCGAUUUCCGGGUGUUGUACGGUUUCUCUUUUUCAG GCUUAUACUCAUGAAUCUUGUCUGAAGCUUUUGAGGGGAGACUUUCAAGUCCUGGAGAA AUAGUAGAUGGCAAGUUUGUGGGUUUUUUUUUUUUACACGAAUUUGAGGAAAACCAAAU GAAUUUGAUAGCCAAAUUGAGACAAUUUCAGCAAAUCUGUAAGCAGUUUGUAUGUUUAG UUGGGGUAAUGAAGUAUUUCAGUUUUGUGAAUAGAUGACCUGUUUUUACUUCCUCACCC UGAAUUCGUUUUGUAAAUGUAGAGUUUGGAUGUGUAACUGAGGCGGGGGGGAGUUUUCA GUAUUUUUUUUUGUGGGGGUGGGGGCAAAAUAUGUUUUCAGUUCUUUUUCCCUUAGGUC UGUCUAGAAUC (SEQ ID NO 3). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 3. In some embodiments, the localization sequence can comprise a sequence encoded by SEQ ID NO: 3. The localization sequence may be transcribed into an RNA molecule.

In some embodiments, the localization sequence that promotes subnuclear localization of the trans-splicing molecule can comprise or consist of sequences from GAS5 long non-coding RNA. The sequence may be a DNA sequence. The sequence may be an RNA sequence. In some embodiments, the sequences from GAS5 can comprise or consist of: GTCTCTTTTCGTAGGATGCCACTCCTGTGAGGTATGGTGCTGGGTGGAGATGCAGTGTGGC TCTGGATAGCACCTATGGACAGTGTGTCCCCAAGGAAGGATGAGAATAGCTACTGAAGTC CTAAAGAGCAAGCCTAACTCAAGCCATTGCGCACACAGGATTAGACAGAAAGCTGGAACGTT GAAATGGTGGAGTCCAACTGCCTGGACCAGGTTAATGGTTCTGCTCCTGGTAACGTTTTTAT CCAAGGATGACTGCTTGGGTAAGGACATGAAGACAGTTTTCCTGTTCATACCTTTTTAAAGGTAT GGAGAGTCGGCTTGACTACACTGTTGTGGACTAAGITITAAAGAAGCAAAGGACTCAGAATT CATGATTGAAGAAATGCAGGCAGACCTGTTATCCTAAACTAGGGTTTTTAATGACCACAACA AGCAAGCATGCAGGTTACTGCTTGAAAGGGTTTTGCCTGAGCCAAGCTAGAGTGCAGTGGCC TTTGAAGCTACTACAGCCTCAAACTTCTTTGGGCTCAAGTGATCCTCAGCCTCCCAGTGGTCTTT TGTAGACTOCCTGATGGAGTCITATGGCACAAGAAGATTAAAACAGTGTGTCCAATTTTAAT AAATTTTTGCAATCCATCAAAAAAAAAAAAAAAAAA (SEQ ID NO: 4). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 4. In some embodiments, the localization sequence can comprise a sequence encoded by SEQ ID NO: 4. The localization sequence may be transcribed into an RNA molecule.

In some embodiments, the localization sequence that promotes subnuclear localization of the trans-splicing molecule can comprise or consists of sequences from a fragment of NEAT1 long non-coding RNA. The sequence may be a DNA sequence. The sequence may be an RNA sequence. In some embodiments, the sequences from a fragment of NEAT1 can comprise or consist of ggtctgggaaggagtgctgtctgcgcctgcctccgctgggcacagcgtggctgcttcaggggctaagcacacactttctgtcttctaaagggccgcca catgccaggagctcaggtgtgagcccggctctggctcttacctcatagggtcactcataggggcacagggagcagaacattgtacecagcgaggcacc acccggcttggeatctgectcggtggacttactacctctagaaggaaatacctgttcctctggcetcagetcctagabtgactggtgtgctgtecctgtta ctccttctgtcaaggtgacaactgtgtgacccatcatctgtgtgtcaaagcaaggccctgcctgggcctctgctcctgtgctgaccccaaaggcaaatgcttt gctagtttccttccagttaatttcatatgaatagatttgtgaaaacttttcaaagccatacctgcacatgmgaacttcaaatttgggtgattcagtgg catccctctaacccccagectccettcecacagaggccaccgtcatggccagttgetgeagtttctttccagagaacctgtgtatgtgtaaagctgtacag gcgtgggtacaccacacagectgtettgcactgtggactgttgagttactagtacatctaggtaagcaccgcatatetgtattcatgtctgcettggtcttttca acatctgtgtggtagccgtinttgaattacccattccctttttggggaaccattaagttgtttcagcuatttttuctgtagataaggctataccgcatutctgtgta catgggtttttatgtacatgggcaagtatatctgtgagagaaaagtttcctcaggaggaattctgggcacagcatgtgtaaatttctaaatatgatggacaccc ccagcttccacctcaaggaggttggteccattgacatttccccacaccttcacccaggctgtgcccttaaacttggttatttgtcaatgtgagaagtggaaaa tagtatttaattgtagtttggatttgtatttctattgg (SEQ II) NO: 44). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 44. In some embodiments, the localization sequence can comprise a sequence encoded by SEQ ID NO: 44. The localization sequence may be transcribed into an RNA molecule.

In some embodiments, the localization sequence that promotes subnuclear localization of the trans-splicing molecule can comprise or consist of sequences from a fragment of NEAT1 long non-coding RNA. In some embodiments, the sequences from a fragment of NEAT1 can comprise or consist of ggttgtatacttactgattaataataagagctctttacatattaaggaaattaacccttttcaaatacattectatttctcactaatctttaagttttattgtaatamtg ctcttttagtttatatatatatgtatatatatatatatgtatatatatatatatacatatatatatacatatatatatactaattttettttatggttccctggattttgtgagtagt ttgaaaaggctaatccagctgaagattttgttgttgttgttaaaccecatgttttctcctaactctttttatttttattttggaggactctatctagacttaattttageat aacaattgacagggttagttagcctgttgtccttacaccattttctggctaatacagctattaactattgatctgtctattcacgtgccagttcctuatggttttac atagtgtaatctgccacttcaaaatagcgaagggaagccctacctcattattctacttttccagaattcttcctggctattecaggetgcatgtttaccttaaccttc cetgtgatgtettcatgccgttgtettcttatgcaagaataaggtacgtetttcratcractcacgtctatttaatttgactttgcattacacagaaagctggtettg gtctgtctacctcggeatctagttgtectcactgccecctagcegaccccaceccatctgactgactaccecatcacagagtacttttatttacgttttgetctg cctaatggttacttgatacctgtcacgccgacagtgtccagttcagtggtctttgcagttgaaatgctcccgtacacactgtccttgttaaaaatgccagtaagttc atacasacccagcttgcacccaaggtcacattc agagagcgtagggctggtgggttgttttccaagcttclgccactgtgtggctagcicttcccactltg gaagttctgtgtacccggaatgtcggagtggagtcctgttctagtgtccagcacctgaccctgtgcccaacccctcaacagcctattcctgctg (SEQ ID NO: 5). In some embodiments, the localization sequence can comprise at least about 60%, about 65%, about 70%, about 75%, about 80%, about 85%, about 90%, about 950, about 97.5%, about 98%, about 99%, or about 100% identity with a sequence encoded by SEQ ID NO: 5. In some embodiments, the localization sequence can comprise a sequence encoded by SEQ ID NO: 5. The localization sequence may be transcribed into an RNA molecule.

In some embodiments, the localization sequence that promotes subnuclear localization of the trans-splicing molecule can comprise or consist of sequences from a fragment of NEAT1 long non-coding RNA. The sequence may be a DNA sequence. The sequence may be an RNA sequence. In some embodiments, the sequences from a fragment of NEAT1 can comprise or consist of ccacagcctgctggaactttttacaaaatatgttgccatgctttaccctgggcactggacataagccccctggcagcctttttcatgtcataaaggggta attgtcctactggtggtctgtaagatgagttagggtgacttgctaatagacattgttmtcttaatatttatgtatgtatmattattaccggttttccatttatgatg gtaatattgtttcttctaagaatatttatttttccttctaaatattgagataaaattcatgcttttgaaatgttctattcagtggcttttagtatatttgctatgttgtgcaa ccatcgacactatccatttctagaactttttcgtcatcccaawatucgctctgtattcataaaaaaataacttcctacctttctctccccctagtctttggtaacc tttgttatactggtuaactttgttgtgctctctgtctgtgtgaatttgcctattctaggggcctcatataaLngtaatcatacagtatttgtctttttgggtctgtctgat ttcacttagcgggttttcagggttcattcatgttgcagcatataacagtactgcgttccttmctggctgttataatattccactgtatggatagaccccattttgttt attcacatcatttggcarttggattatttctggtttttggctattatgaacaatggtgctatgaacagttgcgwwgtttttgtgtgaagcatatgttttcaattc tctcattatatacctaggagtagaattactgggtcatatggtaacctgtatatttttgaggaactgccaaactattttccxacgtccatgcaccatttcacattccca ccagtaagtaatagagggttccaatttctgcgcattettgccaacaetagttattatctgactttctggttataatcattctaatgagtgtgaagtagcetetggtgtc atttggatttgcatttctctgatgagtgatgctatcaagcacetttgctggtgctgttggceatatgtgtatgttccetggagaagtgtct (SEQ ID NO: 6). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 6. In some embodiments, the localization sequence can comprise a sequence encoded by SEQ ID NO: 6. The localization sequence may be transcribed into an RNA molecule.

In some embodiments, the localization sequence that promotes subnuclear localization of the trans-splicing molecule can comprise or consist of sequences from a fragment of MEG3 long non-coding RNA. The sequence may be a DNA sequence. The sequence may be an RNA sequence. In some embodiments, the sequences from a fragment of MEG3 can comprise or consist of: AGCCCCTAGCCTCAGAGCGCAGAGACYCAGACGAGGGACGCCGCCGCCTGGCTCGCTGGCCTGGC GGCGGCTCCTCAGGAGAGGTGGGGCCGCCCACGAGAGGATCCCTCACCCGGGTCCTCCTCAG GGATGACATCATCCGTCCACCTCCTTGTTCTCAAGGACCACCTCCTTCCATGCTGAGCTGCT GCCAAGGGGGCCTGCTGCCCATCTACACCTGACCTAGCACTAGGGAOCACOGTTTGCTGGATC CCACCAACATACAAAGCAGCCACICACTGACCCCCAGCTGAGCAGGATGGCAAAGGATOAAGA GGACCAGGAACTGACCAGCCAGCTGTCCGTCTTACCTAAAGACTTAAACCAATGCCTAGTGA GGGGGCATTTGGGCATTAAGCCCTGACCGTTGCTATGCTCATACTTCTTGACTCTATGAGTACTCT CCTATAAGTCTITGCTTGTGTTCACCTGCTAGCAAACTGGACGTGTTTCCCTCCCCAAGGGGGT GTCAGTGTTGTCGACTGACICTGTCATCACCCTTATTATTGTCGGAATGGAAGGATCCCTTT GCTGAAATTCTCAGGAGGGGGACCGGGCAAGGGCTTGGCCAGCATCCTGCTGGGAACTCC AAGGCCCTGGGTGGGCCTTCTGGAATGAGCATGCTACTGAATCACCAAAGGCACGCCCGACC TCTCTGAAGATCTTCCTATCCTTCTCGGGGAATGGGCTGTCGATGGAGAGCAACCTCCTAGGG TTGUTGTGAGAATAAATGAGATAAAAGAGGCCTCAGGCAGGATCTGGCATAG (SEQ ID NO: 7). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 7. In some embodiments, the localization sequence can comprise a sequence encoded by SEQ ID NO: 7. The localization sequence may be transcribed into an RNA molecule.

In some embodiments, the localization sequence that promotes subnuclear localization of the trans-splicing molecule can comprise or consist of sequences from a fragment of a fragment of NEAT1 long non-coding RNA. The sequence may be a DNA sequence. The sequence may be an RNA sequence. In some embodiments, the sequences from a fragment of a fragment of NEAT1 can comprise or consist of: GAGGAGGTGATCAGCAAATGTTGTTGAAAACGCTGACACTCAGTCCCTTCCCACCCCTC TTGCTTGTCTTACTGTCTTATTTATTCTGAACAGCACTCCAGGCAGCCCTGTCCACGGGC TCTCCTTGCATCAGCCAAGCTTCTGAAAGGCCTGTCTACACTTTGCTCGTCCTTCCTCCTCACCT CCAATTTCCTCTTCAACCCACTGCTTCCTGACTCGCTCTACTCCGTGGAAGCACGCTCACAAA GGCACGTGGGCCGTGGCCCGGCTGGGTCGGCTGAAGAACTGCGGATGGAAGCTGCGGAAGA GGCCCTGATGGGGCCCACCATCCCGGACCCAAGTCTTCTTCCTGGCGGGCCTCTCGTCTCCTT CCTGGTTTGGGCGGAAGCCATCACCTGGATGCCTACGTGGGAAGGGACCTCGAATGTGGGA CCCCAGCCCCTCTCCAGCTCGAAATCCCTCCACAGCCACGGGGACACCCTGCACCTATTCCC ACGGGACAGGCTGGACCCAGAGACTCTGGACCCGGGGCCTCCCCTTGAGTAGAGACCCGCC CTCTGACTGATGGACGCCGCTGACCTGGGGTCAGACCCGTGGGCTGGACCCCTGCCCACCCC GCAGGAACCCTGAGGCCTAGGGGAGCTGTTGAGCCTTCAGTGTCTGCATGTGGGAAGTGGG CTCCTTCACCTACCTCACAGGGCTGTTGTGAGGGGCGCTGTGATGCGGTTCCAAAGCACAGG GCTTGGCGCACCCCACTGTGCTCTCAATAAATGTGTTTCCTGTCTTAACAAA (SEQ ID NO: 8). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 8. In some embodiments, the localization sequence can comprise a sequence encoded by SEQ ID NO: 8. The localization sequence may be transcribed into an RNA molecule.

In some embodiments, the localization sequence that promotes subnuclear localization of the trans-splicing molecule can comprise or consist of sequences from a fragment of PINT1 long non-coding RNA. The sequence may be a DNA sequence. The sequence may be an RNA sequence. In some embodiments, the sequences from a fragment of PINT1 can comprise or consist of: TTGGCCCCACTGTGCACCACACACTCCTTTCCCAGCCCAGGGGCACGCGAACAAAATGAGGC TCAAGCTGACCAGGCCGAGCCGGAGGAACGCTGGGGCTTGGCAGCAGAAGGGATGGGACC AGAGAGAAGGGTGTGGAGGAGACCCCAGTGAGGGCCAGGACATTTCAGGTAAAGAGAGGT CATATCTCCGTACCTCACTTCCTGACACAAACAAGTTTTCACTGTTGTCAGCAACAAAGCCCT AATATAGCTGCGGAAGAGAAAAACTGCATTGCATTTTGCCTCCTGCAAGCATCATCAACAGT TACTGGAGGAACGTAATTCCAGAAAGCTTGAAAGCCGTGGTGATGGTAATTATGTATCAAAT GCCTGGTTCTATTTCTGTTATTATTGTTTTGTCATTTCTGTTTTCCCAGCGATCTGACTGAACT CGCAGAGGGACAAATCCAGTTTTTCTTTTTGACTTTTGTCAAACTAAATCAGGCCTGATAGA AAACTCATTGCTCTCCGGGGAAACAAAGTAGGAGCCACGAAATGTCATTTTAACAGAGCGT GGGTTTGGTGACTGTAGGAAAGGATTTGAGGACGCTCCTTCTGTTCGGCTTCCTATGTCATG AGCACAGGCTCCACGCACGCACAGACACCACGGCTCCCGGATGCTGTGGCTCCCCGATCGG GGCTCCTGCAGCGCCAGAAGCCCCTCCGGGATGCTTCGAGGGGCTCCCGGTGGGTGGAGGT ACGGACGCCGCTGCGGCCGCCGCCGCCAGTCCTGCTGCTGTTGTTGCTGCTGCAGTCACGTG GGAGCCCCTTTAAGTTTCCATAGAGAGGCCTCTCTGGTGTCACATGATGGACATGATATAAT GAAACAACATTGTGGAGAGGAAAGCATTAGGGGAGCCCACGGCTACAAAAACAAGTGAGT GAGAAGAGGTGGGAGGAAGAGAAACTACGCCACCTCCCCTGCAGCCGAGTGCACGCAGCA GCCTGGCGTGACAAGTGGGCGACGCCGGGGGGCAGGGAGCCGGGGTCCTTGGCCCTGGCCG GGG (SEQ ID NO: 9). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 9. In some embodiments, the localization sequence can comprise a sequence encoded by SEQ ID NO: 9. The localization sequence may be transcribed into an RNA molecule.

In some embodiments, the localization sequence that promotes subnuclear localization of the trans-splicing molecule can comprise or consist of sequences from a fragment of PINT1 long non-coding RNA. The sequence may be a DNA sequence. The sequence may be an RNA sequence. In some embodiments, the sequences from a fragment of PINT1 can comprise or consist of: AAATGAAGGAGTTGAGAAGTACATACTCTGAGGTTTATCCTAAATAAACTGAAGTCTAGGC AAGTGGCTGAGCTAGGTTTGCCATGAATCAATCTGCTGAGTATATTTCTTGGTAACTAGTTCA TCTTTCCTTAATTCAGTCAACAAATATGATATCAAGTTTAGTAAGATGATGACAGTTGTCATG TAATACTCCATCTGGAAGATTGTATGCTTCTCAGCTTCCCTAACTTTTCTTAATCTACCTTAA GCTTCCTTTCTGAAAGGTTTCTGTCTTTCCTAACAGTCTCTTCTTTCCGCCAGCTACCTCTTGT GTTTAAGAATTAAAGGAAGATGAGCTAGGAATCAAGAAATACGCAGAACTGATTCCTGTCA CTGGCGCCACCCTGCCACTTGCCCAGAGCTGTGGTATAAATTTTTTTAATAGGCTGAAATAA AAATATGAATAGCATTTTGGTATTAAGCATTAAATTGATAAAGGCTATGAGATACACCTGCT CTCAACCATGTTAATTTTTTATTATTGGTATTAATAAATTATTACTTACTAACATATTAACAA GAATTGCATTGAGAAACAAAGCATCCACAGGCCAAATCTTCTGAATTTCAAATGTTTATATT AATGCGTTGTATTCTAGAAAAGTAGAATTGTCTTAAGTAGCTTTGTAATATAAAGTCATCTAT CAGCCCATTACACCTATTAGAATGTTTTAACCTTTATTTCCCACTTTTTTGTTTCTAGACTGAG TGTACTTGGTCTGGTATTTGTGGCATAACTTACGGAACACATAAGAATGATACAGATACTAT TTAATGATGACCTAATACAAGCTTAGATAGCTAAGGTGAAAGCTTCTATGGCCTTAACATTT TCCTCTTGAAGAATGTATTTTCTGTAATAAAATACAGTGGCTACTTGAAATCTATAAACTTAT GTGAGGTCTGGATAAATCTGAGCAACTTTCTTCTTTGTGCTCCAGGAACCTACGCACTATATA TATAAATAAAGCTTAAGTAAACATCACTGCAAAAAAAAAAAAAAAAA (SEQ ID NO: 10). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 10. In some embodiments, the localization sequence can comprise a sequence encoded by SEQ ID NO: 10. The localization sequence may be transcribed into an RNA molecule.

In some embodiments, the localization sequence that promotes subnuclear localization of the trans-splicing molecule can comprise or consist of sequences from XLOC_009233 long non-coding RNA. The sequence may be a DNA sequence. The sequence may be an RNA sequence. In some embodiments, the sequences from XLOC 009233 can comprise or consist of: AGAGTGAAGGAGCAAAGGGGACTCGGCCGCCATGTTAGGAGTACTGGGACGATTCCGCGGA GCCGGGCAGAGGTTTTAGGGGAATGATTAACAAAGGCGTCCGAAGAAATCGTTGTTGGAAG GTGACCAAGGTGGAAAGAGACGTTGCTTTGGCCCTGCAAGTAAGAAGAGAGAGGGAATAGC CTGAAGGAGTAACACTAAATTTAAAATGACACTTTTTTACCAACCAGCGAAAGCAGATGTTC AAAGGGGATATTGGCCAGAGTCTGTCACACTAAGATGAGAAATGTCCTTTCTTCCTGAAGGT GTCTGATGTGTAAAAATATGATATACTTTGTGCTGTTTCCTCCCTTCCCTTTTGCATATTATTC TGAAACAACATTAACTAGTTACTTTGCGTCATTGAAGGTATGCACTTCCCCTCTATGTTAGGA GTGAATAAAATTAAAAATAGATCCTTATAACAAAGAAAGGCAGATAGAATGATTAAAAATG ACCAAAACATGTTAGAAACAGTCTCTCAGGTGTATGCAGATGGTAATTACAAAAATACTTTT TCAAAAAATGATCTTCTGTGTCATGTTTCTGGGAACAAGTCAAGATGAATGAGTTTGATTTTT AAGCAGAAGTAGTATGTGTTGGTGTCATCCATGAATACAACAATGAAAAAGGC (SEQ ID NO: 11). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 11. In some embodiments, the localization sequence can comprise a sequence encoded by SEQ ID NO: 11. The localization sequence may be transcribed into an RNA molecule.

In some embodiments, the localization sequence that promotes subnuclear localization of the trans-splicing molecule can comprise or consist of sequences from XLOC_003526 long non-coding RNA. The sequence may be a DNA sequence. The sequence may be an RNA sequence. In some embodiments, the sequences from XLOC_003526 can comprise or consist of: ACAATCCAATTCCTAGATGCTTTCTCCAGTTATGGCCCTGACTGGGATCTCCGAAGAATGAC TCAGCTGAAACTATTTCATCATTGCCTACATTTGTCTTCAGACGACCTGCAGTACAAACTTGG CTTAAACCTGCAGTTGACAATGGATTCACCGTGGGAGAATAAATTACAATGGATAAAAAAC AGCATTACAATCCCTCAATAAAATGGGATTTTAACAGACAAAGCTCCTGGTATGTGGTCGTT CTCAGGCCCAAAGGCCCATTTTGCTTTTGGCTGCAACTCTGTAACTCTGTAACAGAGACCAA GTGCCTGCAGGGAGCAGATGTTTGGGGCAGTATCTGCTAAGGTGGCTTTGCTGAAACTTGTT TCTCTCATTGGGTCTTGGAAGTGGCGAATAGGACGTTCCTATAGAAAAACTCAGAAAGAGAC TGTGAAAGATAATCTTAAAGAGGCACTCACGAAACACAAGCTCCTTCTCCGGGAAAAAGAA TCATCACCCTTGTTATCCCATGAAAATTCATCTTCTCCTTTTCAGCTGATATGAGGACTTGAT CTGTCCTTTCCTATAAGTGCCATCTGTGTTTAAATGAGGCTGTTACTCTAACTCAAAGCAGAC TTGCAAGAGTAAAATAATCAATCTTCATCATA (SEQ ID NO: 12). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 12. In some embodiments, the localization sequence can comprise a sequence encoded by SEQ ID NO: 12. The localization sequence may be transcribed into an RNA molecule.

In some embodiments, the localization sequence that promotes subnuclear localization of the trans-splicing molecule can comprise or consist of sequences from a ribozyme. In some embodiments, the ribozyme is the hammerhead ribozyme. The sequence may be a DNA sequence. The sequence may be an RNA sequence. In some embodiments the hammerhead ribozyme can comprise or consist of: aaaaagoggtcaggcagctaaaccaaaaggtttagcaattgectetgatgagtegetgaaatgcgacgaaaaccg (SEQ ID NO: 13). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 13. In some embodiments, the localization sequence can comprise a sequence encoded by SEQ ID NO: 13. The localization sequence may be transcribed into an RNA molecule.

In some embodiments, the localization sequence that promotes subnuclear localization of the trans-splicing molecule can comprise or consist of sequences derived or isolated from a virus. In some embodiments, the virus is a flavivirus. In some embodiments, the virus is a herpesvirus is Kaposi's sarcoma-associated herpesvirus (KSHV). The sequence may be a DNA sequence. The sequence may be an RNA sequence. In some embodiments the KSHV sequence can comprise or consist of: tgttttgTGTTTTGGCTGGGTTTTTCCTTGTTCGCACCGGACACCTCCAGTGACCAGACGGCAAGG TTTTTATCCCAGTGTATATT (SEQ ID NO: 14). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 14. In some embodiments, the localization sequence can comprise a sequence encoded by SEQ ID NO: 14. The localization sequence may be transcribed into an RNA molecule.

In some embodiments, the localization sequence that promotes subnuclear localization of the trans-splicing molecule can comprise or consist of sequences from XLOC_004456 long non-coding RNA. The sequence may be a DNA sequence. The sequence may be an RNA sequence. In some embodiments, the sequences from XLOC_004456 can comprise or consist of: CCCTTCTCCAGAGCGCCGTCGACCCCACGCCACGCAGAGGTGAAGATACGCAGGACGTCCTC TAAAGCAACTTCCGTGGGTGCCAGGTTGTGGGCTGGGGGCGGTGCTGACCTTGAGAGAGCG GAGGAGGCGCAGGCGCAGCACAGTTTAAACTTGATTTTGTGAGGGGACGAACAGCGTGACA ATTAAAAGAAGATCTGGGAATCATCCAATGTGATAATTTATGTGAAATCTTTCTGCAAACTA TACAGTATGATAAAAATATAAGGTAGTTTCACTGGAAACAACAGAAGACTAGAAGCTGATG TGACGTGGCAATATCAACCAGGAATACCCATCTCGAGTTCTGGATATGTGCTCTGAGAAGGT GCCCATGTCACCGACCACACTGGAGGCCAATGCAGATACTGGGGGAAGGTTCCATGGTAGC TAAGTGTGGACAAGCTAATCACTGAAGTTCCCTGATGCAGAGTTGATAATCTCTCTAGAGTT GGATGAAAGACTGTGCTGCCTTGAAGCTCTGAGAGATGCCATGCCAATTCAAGGCAGGGTCT TTATTGTTGAAACTCTTGCGTAATGACATCTTTATTCAGGTGAAAATACAGGATGAATTTCAA CTATATGATATTGTTTATGTTCCTCAGACATGTTATTTGTCTTTACAAAGATTGGTTTCAATA AACTGTGTGACTGATATTAAATAAACATGGAATTTTACACATTCATAATAAAAAAAAAA (SEQ ID NO: 15). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 15. In some embodiments, the localization sequence can comprise a sequence encoded by SEQ ID NO: 15. The localization sequence may be transcribed into an RNA molecule.

In some embodiments, a localization sequence can promote localization of the trans-splicing nucleic acid to the nucleus. In some embodiments, the localization sequence may be derived from a long non-coding RNA. In some embodiments the localization sequence is derived from a long non-coding RNA selected from the list consisting of: JPX, PVT1, NR2F1, and EMX2OS. The sequence may be a DNA sequence. The sequence may be an RNA sequence. In some embodiments, the sequence from JPX can comprise or consist of: UCUGCCUCCCCAGUUCAAGUGGUUGUCCUGCCUCAGCCUCCCGAQUAGCUG (SEQ ID NO: 16). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 16. In some embodiments, the localization sequence can comprise a sequence encoded by SEQ ID NO: 16. The localization sequence may be transcribed into an RNA molecule.

In some embodiments, the sequence (e.g., RNA or DNA sequence) from PVT1 can comprise or consist of: CCCACCUCCCGGGUUCAAGUGAUCCUCCUGCCUCAGCCUCCCGAGUAGCUG (SEQ ID NO: 17). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 17. In some embodiments, the localization sequence can comprise a sequence encoded by SEQ ID NO: 17. The localization sequence may be transcribed into an RNA molecule.

In some embodiments, the sequence (e.g., DNA or RNA sequences) from NR2F1 can comprise or consist of: UCCGCCUCCCGGGUUCAAGUGAUUCUCCUACCUCAGCCUCCUCA (SEQ ID NO: 18). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 18. In some embodiments, the localization sequence can comprise a sequence encoded by SEQ ID NO: 18. The localization sequence may be transcribed into an RNA molecule.

In some embodiments, the sequence (e.g., DNA or RNA sequences) from EMX20S can comprise or consist of: UCCGCCUCCCAGGUUCAAGCGAUUCUCCUGCCUCAACCUCCUGAGUAGCUG (SEQ ID NO: 19). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 19. In some embodiments, the localization sequence can comprise a sequence encoded by SEQ ID NO: 19. The localization sequence may be transcribed into an RNA molecule. In some embodiments, the localization sequence (e.g., DNA or RNA sequences) is derived from a long non coding RNA consensus sequence comprising or consisting of: CGCCUCCCGCGGUUCAAGCGAUUCUCCUGCUCACGCCUCCCGA (SEQ ID NO: 20). In some embodiments, the localization sequence can comprise at least about 60%, about 65%, about 70%, about 75%, about 80%, about 85%, about 90%, about 95%, about 97.5%, about 98%, about 99%, or about 100% identity with a sequence encoded by SEQ ID NO: 20. In some embodiments, the localization sequence can comprise a sequence encoded by SEQ ID NO: 20. The localization sequence may be transcribed into an RNA molecule.

### Intronic Domains

The present disclosure provides a nucleic acid encoding one or more Intronic Domains. The nucleic acid may comprise a DNA encoding the one or more Intronic Domains. The one or more Intronic Domains may be transcribed into RNA. The nucleic acid may comprise an RNA encoding the one or more Intronic Domains. The intronic domain may promote RNA splicing of the Replacement Domain. In some embodiments, the Intronic Domains can carry binding sites that are preferentially-targeted by RNA-binding proteins with disease-causing mutations. In some embodiments, the dissociation constant of these mutated RNA-binding proteins and the Intronic Domain can be lower than the dissociation constant of the non-mutated RNA-binding protein and the Intronic Domain.

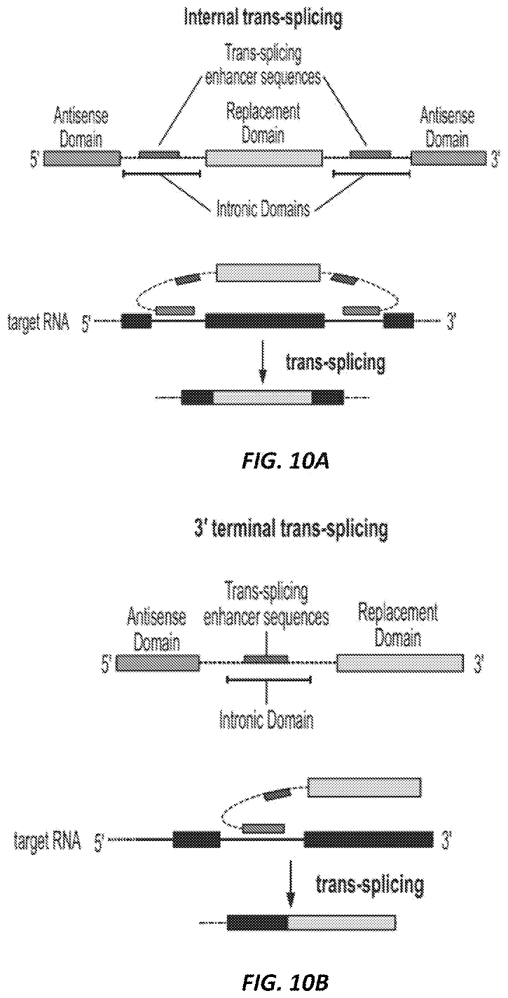

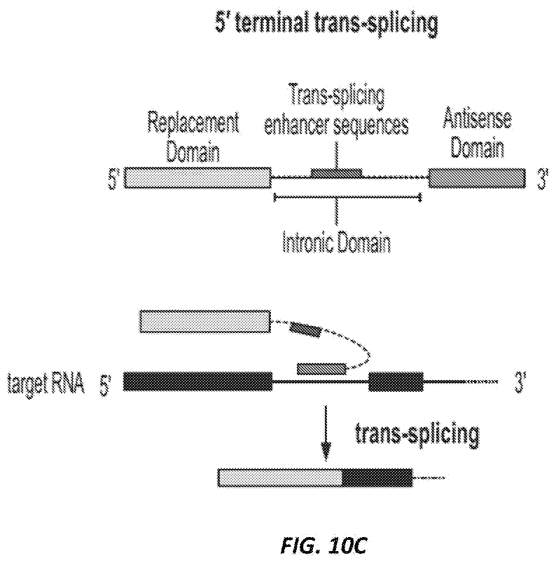

In some embodiments, the Intronic Domains further comprises one or more sequences configured to enhance the trans-splicing of the Replacement Domain. In some embodiments, the one or more sequences configured to enhance the trans-splicing of the Replace Domain can be trans-splicing enhancer sequences. In some embodiments, the one or more sequences may be configured to bind an enzyme staple molecule (ESM). In some embodiments, the ESM may comprise an engineered small nuclear RNA (snRNA). In some embodiments, the one or more sequences can comprise binding sites that are preferentially-targeted by an engineered snRNA. In some embodiments, the engineered small nuclear RNA can be a modified version of U1 snRNA. In some embodiments, this modified U1 snRNA can increase the trans-splicing efficiency of the trans-splicing nucleic acid. FIG. 10A is an example of an embodiment as disclosed herein, and depicts a double trans-splicing molecule which carries two antisense domains, one replacement domain, two intronic domains, and at least two trans-splicing enhancer sequences within the intronic domains. This design promotes replacement of an internal sequence within the target RNA while maintaining the adjacent 5′ and 3′ sequences around the replaced sequence. FIG. 10B illustrates the design of a 3′ terminal trans-splicing RNA that will replace the 3′ terminal end of a target RNA while maintaining the 5′ end. FIG. 10C illustrates the design of a 5′ terminal trans-splicing molecule that will replace the 5′ terminal end of a target RNA while maintaining the 3′ end.

In some embodiments, the trans-splicing enhancer sequences comprise 5′-X₁X₂X₃X₄X₅X₆-3′ wherein X₁ is uracil (U) or guanine (G); X₂ is adenine (A), uracil (U) or guanine (G); X₃ is adenine (A), uracil (U) and guanine (G); X₄ is adenine (A), uracil (U), cytosine (C) or guanine (G); X₅ is adenine (A), cytosine (C), uracil (U) or guanine (G); and X₆ is adenine (A), uracil (U) or guanine (G).

In some embodiments, the trans-splicing enhancer sequences comprise 5′-X₁X₂X₃X₄X₅X₆-3′ wherein: X₁ is selected from the group including adenine (A), uracil (U) and guanine (G): X₂ is selected from the group including adenine (A), uracil (U) and guanine (G); X₃ is selected from the group including adenine (A), uracil (U) and guanine (G); X₄ is selected from the group including adenine (A), uracil (U) and guanine (G); X₅ is selected from the group including adenine (A), uracil (U) and guanine (G); and X₆ is selected from the group including uracil (U) and guanine (G).

In some embodiments, the trans-splicing enhancer sequences comprise 5′-X₁X₂X₃X₄X₅X₆-3′ wherein; X₁ is selected from the group including adenine (A), uracil (U) and guanine (G); X₂ is selected from the group including uracil (U) and guanine (G); X₃ is selected from the group including adenine (A), uracil (U) and guanine (G); X₄ is selected from the group including uracil (U) and guanine (G); X₅ is selected from the group including uracil (U) and guanine (G); and X₆ is selected from the group including uracil (U) and guanine (G).

In some embodiments, the trans-splicing enhancing sequences (trans-splicing enhancer sequences) described herein may include any sequences that promote trans-splicing in an efficient manner. IN some embodiments, trans-splicing enhancer sequences can comprise TTACGG, TAACGG, GGGTTT, GTTTTG, GGTTTT, GGTTTG, GGTTXG, GTAGG, TGGTTG, GGGTAG, GGTAGG, GGTAGT, GTAGTT, GTTGGT, GTGGTT, GGTGGT, TGGTGG, TTGGTG, GTAAGG, TAAGGG, TTAGGCT, TAGGGG, TTGGGG, GTTGGG, GTAGGC, TATFGG, TGTTGG, TATGGCT, TTTGGG, TGTGGG, TTGTGG, GAGTGT, GAGGTA, GGAGGT, TOGGAG, GGGGTG, GGGGGA, GGGGGT, GGGGTA, GGGAGG, GGGTGG, GGAGGG, GGTGGG, GAGGGG, GTGGGG, GAGTGG, GTATGG, GGTATT, GTATTT, GTATTG, AGTTTA, AGGTTA, GTAACG, AGGTAA, GGTAAG, TGGGGG, AGGGTT, AGGTTG, AGGTAG, ATTTTTGG, AGTTGG, TCTGGG, AGAGTG, AGAGGG, AGTGTG, AGAGGT, AGGGAG, AGGGTG, AGGGGG, AGGGGT, AGTGGG, AGTATG, AGGTAT, GTATTC, GGTAAC.

In some embodiments, none, some, or all, of the thymidine bases of the trans-splicing enhancing sequences may be replaced with uracil.

In some embodiments, the Intronic Domains carry binding sites that are preferentially-targeted by RNA-binding proteins with disease-causing mutations. In some embodiments, the dissociation constant of these mutated RNA-binding proteins and the Intronic Domain is lower than the dissociation constant of the non-mutated RNA-binding protein and the Intronic Domain.

### Replacement Domains

The present disclosure provides compositions encoding one or more Replacement Domains. The Replacement Domain may comprise DNA or RNA. The DNA encoding the one or more Replacement Domains can be transcribed into a messenger RNA (mRNA) encoding the one or more Replacement Domains. The Replacement Domain may encode one or more exonic sequences corresponding to a target RNA. The target RNA may comprise a missing or mutated sequence, or portion thereof. The targeting of the exonic sequence to the target RNA may result in trans-splicing of the exonic sequence to the sequence of the target RNA, thereby correcting the target RNA. In some embodiments, the Replacement domain is derived or isolated from the Target RNA. The compositions comprising Replacement Domains disclosed herein includes any strategies where replacement or insertion of RNA sequences can be an effective therapy.

In some embodiments, the Replacement Domain is comprised of sequence derived or isolated from a human gene. In some embodiments of the compositions of the disclosure, the sequence comprising the Replacement Domain has at least 50%, 55%, 60%, 65%, 70%, 75%, 80%, 87%, 90%, 95%, 97%, 99% or any percentage in between of identity with a human gene. In some embodiments, the Replacement Domain has 100% identity with a sequence derived or isolated from a human gene. In some embodiments, the Replacement Domain comprises or consists of 2 nucleotides, 5 nucleotide, 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotide, 250 nucleotides, 260 nucleotides, 270 nucleotides, more than 270 nucleotides, or any number of nucleotides in between.

The Replacement Domains can include, without limitation, sequences derived or isolated from the following genes (with gene accession IDs in brackets and associated diseases in parentheses) such as TNFRSF13B [ENSG00000240505] (common variable immune deficiency), ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C90RF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG000000305K2] (Frontotemporal dementia with parkinsonism); CD1123, MYO7A, US112A [ENSG00000107736, ENSG00000137474, ENSG00000042781] (Usher's syndrome); GALE [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINKI, ATPGAP2 [ENSG00000158828](early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00001150995, ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311](Ataxia-telangiectasia); GLB1 [ENSG00000170266] (GM1 gangliosidosis): GBA [ENSG00000177628] (Gaucher disease); GM2A [ENSG00000196743] (GM2 gangliosidosis); UBE3A [ENSG00000114062](Angelman syndrome); SLC2A1 [ENSG00000117394] (glucose transporter deficiency type 1): LAMP2 [ENSG00000005893] (Danon disease); GLA [ENSG00000102393] (Mabry disease): PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG000001302031 (Familial hypercholesterolemia); MYOC, OPTN, TBK1, WDR36, CYP1B1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1]; IDS [ENSG00000010404](Hunter syndrome or Mucopolysaccharidosis 2): CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170024] (Limb-girdle muscular dystrophy type 2F): DUX4 [ENSG00000260596] (Facioscapulohumeral muscular dystrophy); F9 [ENSG00000101981] (Hemophilia B); F8 [ENSG00000185010] (Hemophilia A): USHHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant hearing impairment); POUF3 [ENSG00000198914] (nonsyndromic hearing loss).

In some embodiments, the Replacement Domain can be codon optimized. In some embodiments, the replacement sequence can be codon optimized that can increase the stability, translation, or other desirable features.

In addition to sequences derived from human genes, Replacement Domains can comprise sequences derived from other organisms in order to alter the stability, translation, processing, or localization of a target RNA. In some embodiments, Replacement Domain derived from non-human sources can include without limitation sequences that increase protein production such as those derived or isolated from Woodchuck Hepatitis Virus (WHY) Post-transcriptional Regulatory Element (WPRE), triplex from MALAT1, the PRE of Hepatitis B virus (HPRE), and an iron response element of the form CAGYCX (Y=U or A; X=U, C, or A).

In some embodiments, the Replacement Domain can be derived or isolated from the Target RNA.

In some embodiments, the Replacement Domain can comprise of sequence derived or isolated from a human gene. In some embodiments, the sequence comprising the Replacement Domain can have at least 50%, 55%, 60%, 65%, 70%, 75%, 80%, 87%, 90%, 95%, 97%, 99% or any percentage in between of identity with a human gene. In some embodiments, the Replacement Domain can have 100% identity with a sequence derived or isolated from a human gene. In some embodiments, the Replacement Domain can comprise 2 nucleotides, 5 nucleotides, 10 nucleotides, 20 nucleotides, 30 nucleotides, 40 nucleotides, 50 nucleotides, 60 nucleotides, 70 nucleotides, 80 nucleotides, 9.0 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between.

### Antisense Domains

The present disclosure provides nucleic acid molecules encoding one or more Antisense Domains. The nucleic acid may comprise RNA. The nucleic acid may comprise DNA. The DNA encoding the one or more Antisense Domains can be transcribed into mRNA encoding the one or more Antisense Domains. An RNA encoding the one or more Antisense Domains may be promoted to a target RNA. In some embodiments, the Antisense Domain can be complementary to the target RNA. In some embodiments, the Antisense Domain can bind to the target RNA. The Antisense Domain may comprise DNA. The DNA comprising an Antisense Domain may encode or be transcribed into an RNA molecule comprising an Antisense Domain. In some embodiments, the RNA molecule comprising an Antisense Domain can be complementary to the target RNA. In some embodiments, the Antisense Domain can bind to the target RNA. In some embodiments of the compositions the present disclosure, a pathogenic RNA molecule can be a target RNA. In some embodiments, the target RNA can comprise a target sequence that is complementary to an Antisense Domain of the trans-splicing nucleic acid the present disclosure.

In some embodiments of the compositions of the disclosure, the sequence comprising the Antisense Domain has at least 50%, 55%, 60%, 65%, 70% 75%, 80%, 87%, 90%, 91%, 92%, 93%, 94%, 95%, 96%, 97%, 98%, 99% or any percentage in between of complementarity to the Target RNA sequence. In some embodiments, the Antisense Domain has 100% complementarity to the Target RNA sequence. In some embodiments, the Antisense Domain comprises or consists of 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotide, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between the complementary to the Target RNA sequence.

In some embodiments, the Antisense Domain is complementary to an RNA transcribed from a gene that is selected from the group consisting of: TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency): HBB [ENSG00000244734] (Beta-thassalemia); HBA1, HBA2 [ENSG00000206172, ENSG00000188536] (alpha-thassalemia); U2AF 1 [ENSG00000160201](myelodysplastic syndrome); SOD1, TARDBP, FUS, MATR3, SOD1, C90RF72 [ENSG0000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism): CDH23, MYO7A, USH2A [ENSG00000107736, ENSG00000137474, ENSG0000042781] (Usher's syndrome); GALC: [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINKI, ATPGAP2 [ENSG00000158828] (early-onset Parkinson's disease); ATXN1, ATXN2, ATXN3, PLEKHG4, SPTBN2, GACNA1A, ATXN7, T113K2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466] (spinocerebellar ataxias): SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG0000075043, ENSG00000184156, ENSG00000084090, ENSG00000163646] (genetic epilepsy disorders); ATM [ENSG00000149311] (Ataxia-telangiectasia); GLB1 [ENSG00000170266] (GM1 gangliosidosis); GBA [ENSG00000177628] (Gaucher disease); GM2A [ENSG00000196743] (GM2 gangliosidosis); UBE3A [ENSG00000114002] (Angelman syndrome); SLC2A1 [ENSG00000117394] (glucose transporter deficiency type 1); LAMP2 [ENSG00000005893] (Dann disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, EN5000000123240, ENSG00000183735, ENSG00000134987, EN5000000138061] (Open Angle Glaucoma): IDUA [ENSG00000127415] (Hurler syndrome or Mucopolysaccharidosis 1): IDS [ENSG00000010404] (blunter syndrome or Mucopolysaccharidosis 2); CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SCYCB [ENSG00000163069] (Limb-girdle muscular dystrophy type 2E); SGCG [ENSG00000102683] (Limb-girdle muscular dystrophy type 3C): SGCD [ENSG00000170624] (Limb-girdle muscular dystrophy type 2F); DUX4 [ENSG00000260596] (Facioscapulohumeral muscular dystrophy): P9 [ENSG00000101981] (Hemophilia B); F8 [ENSG00000185010] (Hemophilia A): USHA2A, RPGR, RP2, RHO, PRPF31, USH1F, PRPF3, PRPF6 [ENSG00000156313, ENSG00000102218, ENSG0000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant heating impairment); POU3F3 [ENSG00000198914] (nonsyndromic hearing loss).

In some embodiments of the compositions and methods the present disclosure, the target sequence can comprise or consists of between 5 and 500 nucleotides. In some embodiments, the target sequence can comprise or consists of between 50 and 250 nucleotides. In some embodiments, the target sequence can comprise or consists of between 5 and 50 nucleotides.

In some embodiments of the compositions and methods the present disclosure, a target sequence can be comprised within a single contiguous stretch of the target RNA. In some embodiments, the target sequence may consist of comprise of one or more nucleotides that are not spread among a single contiguous stretch of the target RNA.

In some embodiments the present disclosure, an Antisense Domain of the present disclosure can bind to a target sequence. In some embodiments the present disclosure, an Antisense Domain of the present disclosure can bind to a target RNA.

In some embodiments the present disclosure, the Antisense Domain can be chosen so that successful trans-splicing causes removal of micro open reading frames in the Target RNA. In this manner, the trans-splicing system can remove micro open reading frames and increases the production of protein from the target RNA.

### Engineered Small Nuclear RNA

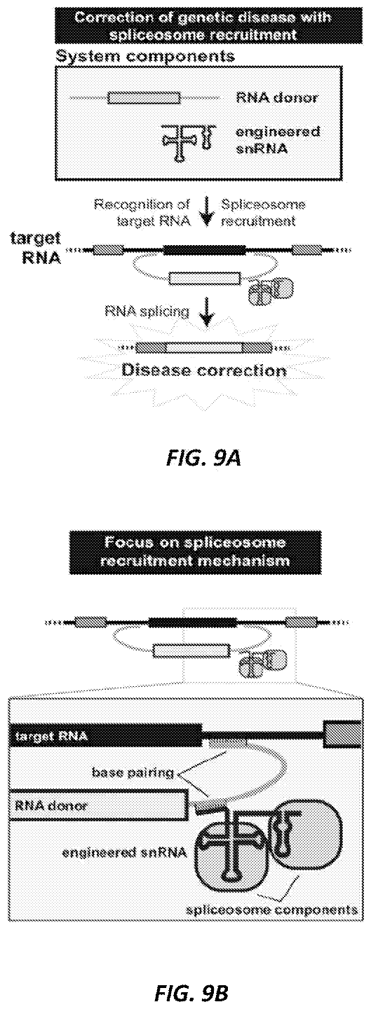

The present disclosure provides an enzyme staple molecule (ESM). A nucleic acid may be provided, comprising one or more domains encoding the ESM. Ile nucleic acid may comprise DNA. The DNA may be transcribed into an RNA, e.g., engineered small nuclear RNA (snRNA). The nucleic acid may comprise an RNA encoding the ESM, e.g., engineered snRNA. The nucleic acid may comprise RNA. In some embodiments, the ESM comprises an engineered snRNA. Ile engineered snRNA may promote RNA splicing of the Replacement Domain. The engineered snRNA may interact with a sequence of the nucleic acid molecule, or a transcribed copy of the nucleic acid molecule, to enhance a trans-splicing of the nucleic acid encoding an exonic sequence. In this manner, the engineered snRNA may promote an association of the exonic sequence with a target RNA, thereby resulting in a trans-splicing of the exonic sequence to the target RNA. An example is provided in FIG. 9. FIG. 9A illustrates a system composed of a donor RNA (e.g., a Replacement Domain encoding an exonic sequence that corresponds to a target RNA sequence or portion thereof) and an engineered small nuclear RNA (esnRNA). The combination of RNA donor molecule and esnRNA correct mutated RNAs via hybridization of the RNA donor to the target RNA carrying a mutation, followed by association of the esnRNA with the RNA donor, results in recruitment of spliceosome components and trans-splicing among the RNA donor molecule and the target RNA. This yields a corrected target RNA with the RNA donor molecule replacing a chosen sequence in the target RNA. FIG. 9B illustrates the how the components interact. Base pairing among the RNA donor and target RNA bring these molecules in close proximity. Base pairing among the esnRNA and the RNA donor brings spliceosome components in close proximity, which may promote a trans-splicing reaction among the target RNA and the RNA donor.

In some embodiments, an engineered snRNA can interact with the intronic Domain to increase the trans-splicing efficiency of the trans-splicing nucleic acid. In some embodiments, the engineered snRNA domain comprise a sequence derived or isolated from a human small nuclear RNA gene. In some embodiments, the human small nuclear RNA gene comprises of U1, U2, U4, U5, U6, U7, U11, and U12 snRNA. In some embodiments, there may be an engineered snRNA sequence that promotes trans-splicing. In some embodiment, the engineered snRNA can be derived or isolated from the human U1 snRNA gene. In some embodiments, the sequences of the engineered snRNA can be derived or isolated from a U1 snRNA variant. In some embodiments of the compositions of the disclosure, the U1 snRNA variant is selected from the list consisting of (name followed by genomic location in brackets according to UCSC human genome assembly 2006): tU1.1 [chr1:16713367-16712967], tU1.2 [chr1:16866030-16865630], vU1.1 [chr1:142438700-142438300], vU1.2 [chr1:142464813-142461413], vU1.4 [chr1:143022739-143022339], vU1.5 [chr1:143202968-143202568], vU1.7 [chr1:144680790 144680390], vU1.8 [chr1:145022927-145022527], vU1.9 [chr1:145977791-145977391], vU1.10 [chr1:146301289-146300889], vU1.11 [chr1:146327427-146327027], vU1.15 [chr1:146871696-146871296], vU1.16 [chr1:147033726-147033326], vU-1.17 [chr1:147460893-147460493], vU1.18 [chr1:147490845-147490445], vU1.19 [chr1:147780880-147780480], tU1.3 [chr1:16939762-16940162], tU1.4 [chr1:17095226-17095626], vU1.3 [chr1:142478876-142479276], vU1.6 [chr1:144094114-144094514], vU1.12 [chr1:146341486-146341886], vU1.13 [chr1:146460770-146461170], vU1.14 [chr1:146608089-146608489], vU1.20 [chr1:147872535-147872935].

### Nucleic Acids

The present disclosure provides nucleic acids for use in compositions and methods as described herein. In some embodiments, the nucleic acid is RNA, DNA, a DNA/RNA hybrid, and/or comprises at least one of a nucleic acid analog, a chemically-modified nucleic acid, or a chimera composed of two or more nucleic acids or nucleic acid analogs. As used herein, the term “nucleic acid analog” refers to a compound having structural similarity to a canonical purine or pyrimidine base occurring in DNA or RNA. The nucleic acid analog may comprise a modified sugar and/or a modified nucleobase, as compared to a purine or pyrimidine base occurring naturally in DNA or RNA. In some embodiments, the nucleic acid analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-meythlguanosine, dihydrouridine, 5-meythylcytidine, C3 spacer, 5-methyl dC, 5-hydroxybutynl-2′-deoxyuridine, 5-nitmindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the nucleic acid analog may be selected from the group consisting of inosine, 7-deaza-2′-deoxyinosine, 2′-aza-2′-deoxyinosine. PNA-inosine, morpholino-inosine, LNA-inosine, phosphoramidate-inosine, 2′-O-methoxyethyl-inosine, and 2′-OMe-inosine. In other embodiments the nucleic acid analog is a nucleic acid mimic (such as, for example, artificial nucleic acids and xeno nucleic acids (XNA). It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical nucleic acid sequence to the reference nucleic acid sequence when compared using sequence identity methods run under default conditions. Specific sequences are provided as examples of particular embodiments. Additionally, an equivalent polynucleotide is one that hybridizes under stringent conditions to the reference polynucleotide or its complement.

In some embodiments, the nucleic acid sequence encoding the trans-splicing nucleic acids comprises a DNA sequence comprising at least about 60%, about 65%, about 70%, about 75%, about 80%, about 85%, about 90%, about 95%, about 97.5%, about 98%, about 99%, or 100% sequence identity to any one of SEQ ID NO: 1-44.

In some embodiments, the nucleic acid sequence can comprise at least about 60%, about 65%, about 70%, about 75%, about 80%, about 85%, about 90%, about 95%, about 97.5%, about 98%, about 99%, or 100% sequences identity with SEQ ID NO: 1. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 2. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 3. In some embodiments, the nucleic acid sequence; can comprise at least about 60%, about 65%, about 70%, about 75%, about 80%, about 85%, about 90%, about 95%, about 97.5%, about 98%, about 99%, or 100% sequence identity with SEQ ID NO: 4. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 5. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 6. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 7. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 8. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 9. In some embodiments, the nucleic acid sequence can comprise at least about 604%, about 65%, about 70%, about 75%, about 80%, about 85%, about 90% about 95%, about 97.5%, about 98%, about 99%, or 100% sequence identity with SEQ ID NO: 10. In some embodiments, the nucleic acid sequence can comprise at least about 60%, about 65%, about 70%, about 75%, about 80%, about 85%, about 904% about 95%, about 97.5%, about 98%, about 99%, or 100% sequence identity with SEQ ID NO: 11. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 12. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 13. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 14. In some embodiments, the nucleic acid sequence can comprise at least about 60, about 65%, about 70%, about 75%, about 80%, about 85%, about 90%, about 95%, about 97.5%, about 98%, about 99%, or 100% sequence identity with SEQ ID NO: 15. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 16. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 17. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 18. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 19. In some embodiments, the nucleic acid sequence can comprise at least about 60%, about 654% about 70%, about 75%, about 80%, about 85%, about 90%, about 95%, about 97.5%, about 98%, about 99%, or 100% sequence identity with SEQ ID NO: 20. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 21. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 22. In some embodiments, the nucleic acid sequence can comprise at least about 60%, about 65%, about 70%, about 75%, about 80%, about 85%, about 94% about 95%, about 97%, about 98%, about 99%, or 100% sequence identity with SEQ ID NO: 23. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 24. In some embodiments, the nucleic acid sequence can comprise at least about 604% about 65%, about 70%, about 75%, about 80%, about 85%, about 90%, about 95%, about 97.5%, about 98%, about 99%, or 100% sequence identity with SEQ ID NO: 25. In some embodiments, the nucleic acid sequence can comprise at least about 60%, about 65% about 70%, about 75%, about 80%, about 85%, about 90%, about 95%, about 97.5%, about 98%, about 99%, or 100% sequence identity with SEQ ID NO: 26. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 27. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 28. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 29. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 30. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 31. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 32. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 33. In some embodiments, the nucleic acid sequence can comprise at least about 60%, about 65%, about 70%, about 75%, about 80%, about 85%, about 90′:%, about 95%, about 97.50, about 98%, about 99%, or 100% sequence identity with SEQ ID NO: 34. In some embodiments, the nucleic acid sequence can comprise at least about 60%, about 65%, about 70%, about 75%, about 80%, about 85%, about 90%, about 95%, about 97.5%, about 98%, about 99%, or 1000% sequence identity with SEQ ID NO: 35. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 36. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 37. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 38. In some embodiments, the nucleic acid sequence can comprise at least about 60%, about 65%, about 70%, about 75%, about 80%, about 854% about 90%, about 954% about 97.5%, about 98%, about 99%, or 100% sequence identity with SEQ ID NO: 39. In some embodiments, the nucleic acid sequence can comprise at least about 60%, about 65%, about 70%, about 75%, about 80%, about 85%, about 90′:%, about 95%, about 97.50, about 98%, about 99%, or 100% sequence identity with SEQ ID NO: 40. In some embodiments, the nucleic acid sequence can comprise at least about 60%, about 65%, about 70%, about 75%, about 80%, about 85%, about 90%, about 95%, about 97.5%, about 98%, about 99%, or 1000% sequence identity with SEQ ID NO: 41. In some embodiments, the nucleic acid sequence can comprise at least about 60%, about 65%, about 70%, about 75%, about 80%, about 85%, about 90% about 95%, about 97.5%, about 98%, about 99%, or 100% sequence identity with SEQ ID NO: 42. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 43. In some embodiments, the nucleic acid sequence can comprise at least about 60%, about 65%, about 70%, about 75%, about 80%, about 85%, about 90%, about 95%, about 97.5%, about 98%, about 99%, or 100% sequence identity with SEQ ID NO: 44.

Also provided herein are nucleic acid sequences encoding the nucleic acids as in compositions and methods as described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical nucleic acid sequence to the reference nucleic acid sequence when compared using sequence identity methods run under default conditions. Specific sequences are provided as examples of particular embodiments. Additionally, an equivalent polynucleotide is one that hybridizes under stringent conditions to the reference polynucleotide or its complement.

The nucleic acid sequences (e.g., polynucleotide sequence) disclosed herein may be codon optimized which is a technique well known in the art. Codon optimization refers to the fact that different cells differ in their usage of particular colons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the an for mammalian cells, as well as for a variety of other organisms. Based on the genetic code, nucleic acid sequences coding for various replacement domains can be generated. In some embodiments, such a sequence is optimized for expression in a host or target cell, such as a host cell used to express the trans-splicing nucleic acid comprising a replacement domain in which the disclosed methods are practiced (such as in a mammalian cell, e.g., a human cell). Codon preferences and codon usage tables for a particular species can be used to engineer isolated nucleic acid molecules encoding a replacement domain (such as one encoding a protein having at least 80%, at least 85%, at least 90%, at least 91%, at least 92%, at least 93%, at least 94%, at least 95%, at least 96%, at least 97%, at least 98%, at least 99%, or 100% sequence identity to its corresponding wild-type protein) that takes advantage of the codon usage preferences of that particular species. For example, the replacement domains disclosed herein can be designed to have codons that are preferentially used by a particular organism of interest. In one example, a replacement domain nucleic acid sequence is optimized for expression in human cells, such as one having at least 70%, at least 80%, at least 85%, at least 90%, at least 91%, at least 924% at least 93%, at least 94%, at least 95%, at least 96%, at least 97%, at least 98%, at least 99% or 1000% sequence identity to its corresponding wild-type or originating nucleic acid sequence. In some embodiments, an isolated trans-splicing nucleic acid molecule encoding at least one replacement domain (which can be part of a vector) can include at least one replacement domain coding sequence that is codon optimized for expression in a eukaryotic cell, or at least one replacement domain coding sequence codon optimized for expression in a human cell. In one embodiment, such a codon optimized replacement domain coding sequence has at least 80%, at least 85%, at least 904% at least 91%, at least 92%, at least 93%, at least 94%, at least 95%, at least 96%, at least 97%, at least 98%, at least 99% or 100% sequence identity to its corresponding wild-type or originating sequence. In another embodiment, a eukaryotic cell codon optimized nucleic acid sequence encodes a replacement domain having at least 80%, at least 85%, at least 90%, at least 91%, at least 92%, at least 931, at least 94%, at least 95%, at least 96%, at least 97%, at least 98%, at least 99%, or 100% sequence identity to its corresponding wild-type or originating protein. In another embodiment, a variety of clones comprising functionally equivalent nucleic acids may be routinely generated, such as nucleic acids which differ in sequence, but which encode the same replacement domain protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC: aspartic acid can be encoded by GAT or GAC; cysteine can be encoded by TOT or TGC: alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 19K8, Biochemistry, 3.sup.rd Edition, W.H.5 Freeman and Co., NY, which is incorporated herein by reference in its entirety).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogsteen binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme. Examples of stringent hybridization conditions include: incubation temperatures of about 25° C. to about 37° C.: hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 6K° C.; buffer concentrations of about 1×SSC to about 0.1×SSC.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, the trans-splicing nucleic acid further can comprise a 5′ untranslated region. In some embodiments, the 5′ untranslated region can increase the stability of the trans-splicing nucleic acid. In some embodiments, the 5′ untranslated region can alter the localization of the trans-splicing nucleic acid. In some embodiments, the 5′ untranslated region can alter the processing of the trans-splicing nucleic acid.

In some embodiments, the trans-splicing nucleic acid further can comprise a 3′ untranslated region. In some embodiments, the 3′ untranslated region can increase the stability of the trans-splicing nucleic acid. In some embodiments, the 3′ untranslated region can alter the localization of the trans-splicing nucleic acid. In some embodiments, the 3′ untranslated region can alter the processing of the trans-splicing nucleic acid.

In some embodiments of the compositions of the present disclosure, the sequence encoding the trans-splicing nucleic acid further can comprise a sequence encoding a promoter capable of expressing the trans-splicing nucleic acid in a eukaryotic cell.

### Sequences of ALB-Targeting Trans-Splicing Molecules

In some embodiments, the systems, methods, and composition described herein can be used to deliver a nucleic acid encoding a replacement gene. In some embodiments, the replacement gene can be ATP7B, a gene that is primarily expressed in the liver and mutated in Wilson's disease. For example, by trans-splicing the ATP7B coding sequence into a liver-specific and highly-expressed gene such as ALB, the ATP7B gene expression can be generated in the liver only. In some embodiments, various lncRNA sequences can influence the activity of a trans-splicing nucleic acid that targets the human gene. In some embodiments, the human gene can be ALB.

In some embodiments, a trans-splicing molecule denoted P1779 can comprise a sequence (e.g., DNA sequence or RNA sequence) derived from the lncRNA GAS5 along with the full length coding sequence of the ATP7B gene, an antisense region that targets ALB, and a splicing domain: gccaccATGCCTGAGCAGGAGAGACAGATCACCGCACGTgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGGAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc getGCAGCCGACGACGATGGGGACAAGTGGAGTCTCCTGTTGAATGGTCGAGACGAAGAACA ATATATCgactacaaagaccatgacggtgattataaagatcatgacaicgactataaggatgaegatgacaaaggetecggegagggcagggga agtettctaacatgeggggaegtggaggaaaateceggeccateATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgeggecgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAGTCTTTTCGAGGTAGGAGTCG ACTCCTGTGAGGTATGGTGCTGGGTGCAGATGCAGTGTGGCTCTGGATAGCACCTTATGGAC AGTTGTGTCCCCAAGGAAGGATGAGAATAGCTACTGAAGTCCTAAAGAGCAAGCCTAACTC AAGCCATTGGCACACAGGCATTAGACAGAAAGCTGGAAGTTGAAATGGTGGAGTCCAACTT GCCTGGACCAGCTTAATGGTTCTGCTCCTGGTAACGTTTTTATCCATGGATGACTTGCTTGGG TAAGGACATGAAGACAGTTCCTGTCATACCTTTTAAAGGTATGGAGAGTCGGCTTGACTACA CTGTGTGGAGCAAGTTTTAAAGAAGCAAAGGACTCAGAATTCATGATTGAAGAAATGCAGG CAGACCTGTTATCCTAAACTAGGGTTTTTAATGACCACAACAAGCAAGCATGCAGCTTACTG CTTGAAAGGGTCTTGCCTCACCCAAGCTAGAGTGCAGTGGCCTTTGAAGCTTACTACAGCCT CAAACTTCTGGGCTCAAGTGATCCTCAGCCTCCCAGTGGTCTTTGTAGACTGCCTGATGGAG TCTCATGGCACAAGAAGATTAAAACAGTGTCTCCAATTTTAATAAATTTTTGCAATCCATCA AAAAAAAAAAAAAAAAA (SEQ ID NO: 21). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 21. In some embodiments, the trans-splicing molecule denoted P1779 can comprise a sequence encoded by SEQ ID NO: 21. The trans-splicing molecule denoted P1779 may be transcribed into an RNA molecule.

The trans-splicing molecule denoted P1780 can comprise a sequence (e.g., DNA or RNA sequence) derived from the lncRNA NEAT1 along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAggtctgggaaggagtgctgtctgcgcctgccctc cgctgggcacagcgtggctgcttcaggggctaagcacacactttctgtcttctaaagggccgccacatgccaggagctcaggtgtgagcccggctctgg ctcttacctcatagggtcactcataggggcacagggagcagaacattgtacacagcgaggcaccacccggcttggcatctgcctcggtggacttactac ctctagaaggaaatacctgagttcctctggcctcagctcctagagtgactggtgtgctgtccctgttactcttctgtcaaggtgacaactgtgtgacccatcat ctgtgtgtcaaagcaaggccctgcctgggcctctgctcctgtgctgaccccaaaggcaaatgctttgctagtttccttccagttaafttcacctatgaatagat gtgtgaaaactgttcaaagccatacctgcacatgtttgaacttcaaaccctgtgggtgattcagtggcatctttctctaacccccagcctcccttcocacaga ggccaccgtcatggccagttgctgcagtttctttccagagaacctgtgtatgtgtaaagctgtacaggcgtgggtacaccacacagcctgtcttgcactgtg gactgttgagttactagtacatctaggtaagcaccgcatatctgtattcatgtctgccftggtcttttcaacatctgtgtggtagccgtgtttgaattacccattcc ctttttggggaaccattaagttgtttcagcaatttttactgtagataaggctataccgcatatctgtgtacatgggtttttatgtacatgggcaagtatatctgtga gagaaaagtttcctcaggaggaattctgggcacagcatgtgtaaatttctaaatatgatggacacccccagcttccacctcaaggaggttggtcccattgac atttccccacaccttcacccaggctgtgcccttaaacttggttatttgtcaatgtgagaagtggaaaatagtatttaattgtagtttggatttgtatttctattgg (SEQ ID NO: 22). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 22. In some embodiments, the trans-splicing molecule denoted P1779 can comprise a sequence encoded by SEQ ID NO: 22. The trans-splicing molecule denoted P1779 may be transcribed into an RNA molecule.

The trans-splicing molecule denoted P1781 can comprise a sequence (e.g., DNA or RNA sequence) derived from the lncRNA NEAT1 along with the full length coding sequence of the ATP7B gene, an antisense region that targets ALB, and a splicing domain: gccaccATGCCTGAGCAGGAGAGACAGATCACCGCACGLAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAggttgtatacttactgattaataataagagctctttac atattaaggaaattaacccttttcaaatacattcctatttctcactaatctttaagttttattgtaatattttgctctttagtttatatatatatgtatatatatatatatgtat atatatatatatacatatatatatacatatatatatactaattttcttttatggttcctggattttgtgagtagtttgaaaaggctaatccagctgaagaftttgttgttgt tgttaaaccccatgttttctcctaactctttttatttttattttggaggactctatctagacttaattttagcataacaagtgacagggttagttagcctgttgtccttac accattttctggctaatacagctattaactattgatctgtctattcacgtgccagttcctaatggttttacatagtgtaatctgcacttcaaaatagcgaagggaa gccctacctcattattctacttttccagaattctcctggctattccaggctgcatgtttaccttaaccttccctgtgatgtcttcatgccgttgtcttcttatgcaaga ataaggtacgtctttccatccactcacgtctatttaatttgactttgcattacacagaaagctggtcttggtctgtctacctcggcatctagttgtcctcactgccc cctagccgaccccaccccatctgactgactaccccatcacagagtacttttatttacgttttgctctgcctaatggttacttgatactgtcacgccgacagtgtc cagttcagtggtctttgcagttgaaatgctcccgtacacactgtcttgttaaaaatgccagtaagttcatacaaacccagcttgcacccaaggtcacattcag agagcgtagggctgggatgggttgttttccaagctfctgccactgtgtggctagctcttcccactgggaagttctgtgtacccggaatgtcggagtggagtc ctgttctagtgtccagcacctgaccctgtgcccaacccctcaacagcctattcctgctg (SEQ ID NO: 23). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 23. In some embodiments, the trans-splicing molecule denoted P1779 can comprise a sequence encoded by SEQ ID NO: 23. The trans-splicing molecule denoted P1779 may be transcribed into an RNA molecule.

The trans-splicing molecule denoted P1782 can comprise a sequence (e.g., DNA or RNA sequence) derived from the lncRNA NEAT1 along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGGGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccacagcctgctggaactttttacaaaatatgttgc catgctggaccctgggcactggacataagccccctggcagcctttttcatgtcacccaaaggggtaattgtcctactggtggtctgtaagatgagttagggt gacttgctaatagacattgtaaatcttaatatttatgtatgtatttfattattaccggtfttccatttatgatggfaatattgtttcttctaagaatatttatttttccttctaa atattgagataaaattcatgcttttgauatgttctattcagtggcttttagtatatttgctatgttgtgcaaccatcgacactatccatttctaguactttttogtcatc ccaaacagacgctctgtattcataaaaaaataacttcctacctgtctctccccctagtctttggtaacctttgttatactggtaaactttgttgtgctctctgtctgt gtgaatttgcctattctaggggcctcatataagtgtaatcatacagtatttgtctttttgggtctgtctgatttcacttagcgggttttcagggttcattcatgttgca gcatataacagtactgcgttcctttttctggctgaataatattccactgtatggatagaccccattttgtttattcacacatcatttggacatttggattatttctggtt tttggctattatgaacaatggtgctatguacagttgcgtacaagtttttgtgtgaacatatgttttcaattctctcattatatacctaggagtagaattactgggtca tatggtaactgtatatttttgaggaactgccaaactattttcccacgtccatgcaccatttcacattcccaccagtaagtaagagggttccaatttctgcgcattc ttgccaacactagttattatctgactttctggttataatcattctaatgagtgtgaagtagcctctggtgtcatttggatttgcatttctctgatgagtgatgctatca agcacctttgctggfgctgttggccatatgtgtatgttccctggagaagtgtct (SEQ ID NO: 24). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 24. In some embodiments, the trans-splicing molecule denoted P1779 can comprise a sequence encoded by SEQ ID NO: 24. The trans-splicing molecule denoted P1779 may be transcribed into an RNA molecule.

The trans-splicing molecule denoted P1783 can comprise a sequence (e.g., DNA or RNA sequence) derived from the lncRNA MEG3 along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CCCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTGGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTACCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgaoggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAAGCCCCTAGCGCAGACGGCGG AGAGCAGAGAGGGAGCGCGCCTTGGCTCGCTGGCCTTGGCGGCGGCTCCTCAGGAGAGCTG GGGCGCCCACGAGAGGATCCCTCACCCGGGTCTCTCCTCAGGGATGACATCATCCGTCCACC TCCTTGTCTTCAAGGACCACCTCCTCTCCATGCTGAGCTGCTGCCAAGGGGCCTGCTGCCCAT CTACACCTCACGAGGGCACTAGGAGCACGGTTTCCTGGATCCCACCAACATACAAAGCAGC CACTCACTGACCCCCAGGACCAGGATGGCAAAGGATGAAGAGGACCGGAACTGACCAGCCA GCTGTCCCTCTTACCTAAAGACTTAAACCAATGCCCTAGTGAGGGGGCATTGGGCATTAAGC CCTGACCTTTGCTATGCTCATACTTTGACTCTATGAGTACTTTCCTATAAGTCTTTGCTTGTGT TCACCTGCTAGCAAACTGGAGTGTTTCCCTCCCCAAGGGGGTGTCAGTCTTTGTCGACTGACT CTGTCATCACCCTTATGATGTCCTGAATGGAAGGATCCCTTTGGGAAATTCTCAGGAGGGGG ACCTGGGCCAAGGGCTTGGCCAGCATCCTGCTGGCAACTCCAAGGCCCTGGGTGGGCTTCTG GAATGAGCATGCTACTGAATCACCAAAGGCACGCCCGACCTCTCTGAAGATCTTCCTATCCT TTTCTGGGGGAATGGGGTCGATGAGAGCAACCTCCTAGGGTTGTTGTGAGAATTAAATGAGA TAAAAGAGGCCTCAGGCAGGATCTGGCATAG (SEQ ID NO: 25). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 25. In some embodiments, the trans-splicing molecule denoted P1779 can comprise a sequence encoded by SEQ ID NO: 25. The trans-splicing molecule denoted P1779 may be transcribed into an RNA molecule.

The trans-splicing molecule denoted P1784 can comprise a sequence (e.g., DNA or RNA sequence) derived from the lncRNA MEG2 along with the full length coding sequence of the ATP7B gene, an antisense region that targets ALB, and a splicing domain: gccaccATGCCTGAGCAGGAGAGACAGATCACCGCACGLAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAGAGGAGGTGATCAGCAAATGT TTGTTGAAAAGGTTTGACAGGTCAGTCCCTTCCCACCCCTCTTGCTTGTCTTACTTGTCTTATT TATTCTCCAACAGCACTCCAGGCAGCCCTTGTCCACGGGCTCTCCTTGCATCAGCCAAGCTTC TTGAAAGGCCTGTCTACACTTGCTGTCTTCCTTCCTCACCTCCAATTTCCTCTTCAACCCACTG CTTCCTGACTCGCTCTACTCCGTGGAAGCACGCTCACAAAGGCACGTGGGCCGTGGCCCGGC TGGGTCGGCTGAAGAACTGCGGATGGAAGCTGCGGAAGAGGCCCTGATGGGGCCCACCATC CCGGACCCAAGTCTTCTTCCTGGCGGGCCTCTCGTCTCCTTCCTGGTTTGGGCGGAAGCCATC ACCTGGATGCCTACGTGGGAAGGGACCTCGAATGTGGGACCCCAGCCCCTCTCCAGCTCGAA ATCCCTCCACAGCCACGGGGACACCCTGCACCTATTCCCACGGGACAGGCTGGACCCAGAG ACTCTGGACCCGGGGCCTCCCCTTGAGTAGAGACCCGCCCTCTGACTGATGGACGCCGCTGA CCTGGGGTCAGACCCGTGGGCTGGACCCCTGCCCACCCCGCAGGAACCCTGAGGCCTAGGG GAGCTGTTGAGCCTTCAGTGTCTGCATGTGGGAAGTGGGCTCCTTCACCTACCTCACAGGGC TGTTGTGAGGGGCGCTGTGATGCGGTTCCAAAGCACAGGGCTTGGCGCACCCCACTGTGCTC TCAATAAATGTGTTTCCTGTCTTAACAAA (SEQ ID NO: 26). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 26. In some embodiments, the trans-splicing molecule denoted P1779 can comprise a sequence encoded by SEQ ID NO: 26. The trans-splicing molecule denoted P1779 may be transcribed into an RNA molecule.

The trans-splicing molecule denoted P1785 can comprise a sequence (e.g., DNA or RNA sequence) derived from the lncRNA PINT along with the full length coding sequence of the ATP7B gene, an antisense region that targets ALB, and a splicing domain: gccaccATGCCTGAGCAGGAGAGACAGATCACCGCACGTgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctocggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTATTGGCCCCACTGTGCACCACA CACTCCTTTCCCAGCCCAGGGGCACGCGAACAAAATGAGGCTCAAGCTGACCAGGCCGAGC CGGAGGAACGCTGGGGCTTGGCAGCAGAAGGGATGGGACCAGAGAGAAGGGTGTGGAGGA GACCCCAGTGAGGGCCAGGACATTTCAGGTAAAGAGAGGTCATATCTCCGTACCTCACTTCC TGACACAAACAAGTTTTCACTGTTGTCAGCAACAAAGCCCTAATATAGCTGCGGAAGAGAA AAACTGCATTGCATTTTGCCTCCTGCAAGCATCATCAACAGTTACTGGAGGAACGTAATTCC AGAAAGCTTGAAAGCCGTGGTGATGGTAATTATGTATCAAATGCCTGGTTCTATTTCTGTTAT TATTGTTTTGTCATTTCTGTTTTCCCAGCGATCTGACTGAACTCGCAGAGGGACAAATCCAGT TTTTCTTTTTGACTTTTGTCAAACTAAATCAGGCCTGATAGAAAACTCATTGCTCTCCGGGGA AACAAAGTAGGAGCCACGAAATGTCATTTTAACAGAGCGTGGGTTTGGTGACTGTAGGAAA GGATTTGAGGACGCTCCTTCTGTTCGGCTTCCTATGTCATGAGCACAGGCTCCACGCACGCA CAGACACCACGGCTCCCGGATGCTGTGGCTCCCCGATCGGGGCTCCTGCAGCGCCAGAAGCC CCTCCGGGATGCTTCGAGGGGCTCCCGGTGGGTGGAGGTACGGACGCCGCTGCGGCCGCCG CCGCCAGTCCTGCTGCTGTTGTTGCTGCTGCAGTCACGTGGGAGCCCCTTTAAGTTTCCATAG AGAGGCCTCTCTGGTGTCACATGATGGACATGATATAATGAAACAACATTGTGGAGAGGAA AGCATTAGGGGAGCCCACGGCTACAAAAACAAGTGAGTGAGAAGAGGTGGGAGGAAGAGA AACTACGCCACCTCCCCTGCAGCCGAGTGCACGCAGCAGCCTGGCGTGACAAGTGGGCGAC GCCGGGGGGCAGGGAGCCGGGGTCCTTGGCCCTGGCCGGGG (SEQ ID NO: 27). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 27. In some embodiments, the trans-splicing molecule denoted P1779 can comprise a sequence encoded by SEQ ID NO: 27. The trans-splicing molecule denoted P1779 may be transcribed into an RNA molecule.

The trans-splicing molecule denoted P1786 can comprise a sequence (e.g., DNA or RNA sequence) derived from the lncRNA PINT along with the full length coding sequence of the ATP7B gene, an antisense region that targets ALB, and a splicing domain: gccaccATGCCTGAGCAGGAGAGACAGATCACCGCACGLAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG CAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAAAATGAAGGAGTTGAGAAGTA CATACTCTGAGGTTTATCCTAAATAAACTGAAGTCTAGGCAAGTGGCTGAGCTAGGTTTGCC ATGAATCAATCTGCTGAGTATATTTCTTGGTAACTAGTTCATCTTTCCTTAATTCAGTCAACA AATATGATATCAAGTTTAGTAAGATGATGACAGTTGTCATGTAATACTCCATCTGGAAGATT GTATGCTTCTCAGCTTCCCTAACTTTTCTTAATCTACCTTAAGCTTCCTTTCTGAAAGGTTTCT GTCTTTCCTAACAGTCTCTTCTTTCCGCCAGCTACCTCTTGTGTTTAAGAATTAAAGGAAGAT GAGCTAGGAATCAAGAAATACGCAGAACTGATTCCTGTCACTGGCGCCACCCTGCCACTTGC CCAGAGCTGTGGTATAAATTTTTTTAATAGGCTGAAATAAAAATATGAATAGCATTTTGGTA TTAAGCATTAAATTGATAAAGGCTATGAGATACACCTGCTCTCAACCATGTTAATTTTTTATT ATTGGTATTAATAAATTATTACTTACTAACATATTAACAAGAATTGCATTGAGAAACAAAGC ATCCACAGGCCAAATCTTCTGAATTTCAAATGTTTATATTAATGCGTTGTATTCTAGAAAAGT AGAATTGTCTTAAGTAGCTTTGTAATATAAAGTCATCTATCAGCCCATTACACCTATTAGAAT GTTTTAACCTTTATTTCCCACTTTTTTGTTTCTAGACTGAGTGTACTTGGTCTGGTATTTGTGG CATAACTTACGGAACACATAAGAATGATACAGATACTATTTAATGATGACCTAATACAAGCT TAGATAGCTAAGGTGAAAGCTTCTATGGCCTTAACATTTTCCTCTTGAAGAATGTATTTTCTG TAATAAAATACAGTGGCTACTTGAAATCTATAAACTTATGTGAGGTCTGGATAAATCTGAGC AACTTTCTTCTTTGTGCTCCAGGAACCTACGCACTATATATATAAATAAAGCTTAAGTAAAC ATCACTGCAAAAAAAAAAAAA (SEQ ID NO: 28). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 28. In some embodiments, the trans-splicing molecule denoted P1779 can comprise a sequence encoded by SEQ ID NO: 28. The trans-splicing molecule denoted P1779 may be transcribed into an RNA molecule.

The trans-splicing molecule denoted P1787 can comprise a sequence (e.g., DNA or RNA sequence) derived from the lncRNA XLOC_003526 along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTG GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctCCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAACAATCCAATTCCTAGATGCT TTCTCCAGTTATGGCCCTGACTGGGATCTCCGAAGAATGACTCAGCTGAAACTATTTCATCAT TGCCTACATTTGTCTTCAGACGACCTGCAGTACAAACTTGGCTTAAACCTGCAGTTGACAAT GGATTCACCGTGGGAGAATAAATTACAATGGATAAAAAACAGCATTACAATCCCTCAATAA AATGGGATTTTAACAGACAAAGCTCCTGGTATGTGGTCGTTCTCAGGCCCAAAGGCCCATTT TGCTTTTGGCTGCAACTCTGTAACTCTGTAACAGAGACCAAGTGCCTGCAGGGAGCAGATGT TTGGGGCAGTATCTGCTAAGGTGGCTTTGCTGAAACTTGTTTCTCTCATTGGGTCTTGGAAGT GGCGAATAGGACGTTCCTATAGAAAAACTCAGAAAGAGACTGTGAAAGATAATCTTAAAGA GGCACTCACGAAACACAAGCTCCTTCTCCGGGAAAAAGAATCATCACCCTTGTTATCCCATG AAAATTCATCTTCTCCTTTTCAGCTGATATGAGGACTTGATCTGTCCTTTCCTATAAGTGCCA TCTGTGTTTAAATGAGGCTGTTACTCTAACTCAAAGCAGACTTGCAAGAGTAAAATAATCAA TCTTCATCATA (SEQ ID NO: 29). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 29. In some embodiments, the trans-splicing molecule denoted P1779 can comprise a sequence encoded by SEQ ID NO: 29. The trans-splicing molecule denoted P1779 may be transcribed into an RNA molecule.

The trans-splicing molecule denoted P1788 can comprise a sequence (DNA or RNA sequence) derived from the lncRNA XLOC_009233 along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTGCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctcoggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAAGAGTGAAGGAGCAAAGGGG ACTCGGCCGCCATGTTAGGAGTACTGGGACGATTCCGCGGAGCCGGGCAGAGGTTTTAGGG GAATGATTAACAAAGGCGTCCGAAGAAATCGTTGTTGGAAGGTGACCAAGGTGGAAAGAGA CGTTGCTTTGGCCCTGCAAGTAAGAAGAGAGAGGGAATAGCCTGAAGGAGTAACACTAAAT TTAAAATGACACTTTTTTACCAACCAGCGAAAGCAGATGTTCAAAGGGGATATTGGCCAGAG TCTGTCACACTAAGATGAGAAATGTCCTTTCTTCCTGAAGGTGTCTGATGTGTAAAAATATG ATATACTTTGTGCTGTTTCCTCCCTTCCCTTTTGCATATTATTCTGAAACAACATTAACTAGTT ACTTTGCGTCATTGAAGGTATGCACTTCCCCTCTATGTTAGGAGTGAATAAAATTAAAAATA GATCCTTATAACAAAGAAAGGCAGATAGAATGATTAAAAATGACCAAAACATGTTAGAAAC AGTCTCTCAGGTGTATGCAGATGGTAATTACAAAAATACTTTTTCAAAAAATGATCTTCTGT GTCATGTTTCTGGGAACAAGTCAAGATGAATGAGTTTGATTTTTAAGCAGAAGTAGTATGTG TTGGTGTCATCCATGAATACAACAATGAAAAAGGC (SEQ ID NO: 30). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 30. In some embodiments, the trans-splicing molecule denoted P1779 can comprise a sequence encoded by SEQ ID NO: 30. The trans-splicing molecule denoted P1779 may be transcribed into an RNA molecule.

The trans-splicing molecule denoted P1789 can comprise a sequence (e.g., DNA or RNA sequence) derived from the lncRNA XLOC 004456 along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTGGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA TTATTATAATAATGAGCACACTGACTCTTGTGGTATGGATTGTTATCGGCTTTATCGATTTCG GAGTCGTACAAAAATATTTGCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTACCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggg agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTACCCTTCTCCAGAGCGCCGTCG ACCCCACGCCACGCAGAGGTGAAGATACGCAGGACGTCCTCTAAAGCAACTTCCGTGGGTG CCAGGTTGTGGGCTGGGGGGGGTGCTGACCTTGAGAGAGCGGAGGAGGCGCAGGCGCAGCA CAGTTTAAACTTGATTTTGTGAGGGGACGAACAGCGTGACAATTAAAAGAAGATCTGGGAA TCATCCAATGTGATAATTTATGTGAAATCTTTCTGCAAACTATACAGTATGATAAAAATATA AGGTAGTTTCACTGGAAACAACAGAAGACTAGAAGCTGATGTGACGTGGCAATATCAACCA GGAATACCCATCTCGAGTTCTGGATATGTGCTCTGAGAAGGTGCCCATGTCACCGACCACAC TGGAGGCCAATGCAGATACTGGGGGAAGGTTCCATGGTAGCTAAGTGTGGACAAGCTAATC ACTGAAGTTCCCTGATGCAGAGTTGATAATCTCTCTAGAGTTGGATGAAAGACTGTGCTGCC TTGAAGCTCTGAGAGATGCCATGCCAATTCAAGGCAGGGTCTTTATTGTTGAAACTCTTGCG TAATGACATCTTTATTCAGGTGAAAATACAGGATGAATTTCAACTATATGATATTGTTTATGT TCCTCAGACATGTTATTTGTCTTTACAAAGATTGGTTTCAATAAACTGTGTGACTGATATTAA ATAAACATGGAATTTTACACATTCATAATAAAAAAAAAA (SEQ ID NO: 31). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 31. In some embodiments, the trans-splicing molecule denoted P1779 can comprise a sequence encoded by SEQ ID NO: 31. The trans-splicing molecule denoted P1779 may be transcribed into an RNA molecule.

The trans-splicing molecule denoted P1790 can comprise a sequence (e.g., DNA or RNA sequence) derived from the lncRNA along with the full length coding sequence of the ATP7B gene, an antisense region that targets ALB, and a splicing domain: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctCCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaGTCTT TTCGAGGTAGGAGTCGACTCCTGTGAGGTATGGTGCTGGGTGCAGATGCAGTGTGGCTCTGG ATAGCACCTTATGGACAGTTGTGTCCCCAAGGAAGGATGAGAATAGCTACTGAAGTCCTAA AGAGCAAGCCTAACTCAAGCCATTGGCACACAGGCATTAGACAGAAAGCTGGAAGTTGAAA TGGTGGAGTCCAACTTGCCTGGACCAGCTTAATGGTTCTGCTCCTGGTAACGTTTTTATCCAT GGATGACTTGCTTGGGTAAGGACATGAAGACAGTTCCTGTCATACCTTTTAAAGGTATGGAG AGTCGGCTTGACTACACTGTGTGGAGCAAGTTTTAAAGAAGCAAAGGACTCAGAATTCATG ATTGAAGAAATGCAGGCAGACCTGTTATCCTAAACTAGGGTTTTTAATGACCACAACAAGCA AGCATGCAGCTTACTGCTTGAAAGGGTCTTGCCTCACCCAAGCTAGAGTGCAGTGGCCTTTG AAGCTTACTACAGCCTCAAACTTCTGGGCTCAAGTGATCCTCAGCCTCCCAGTGGTCTTTGTA GACTGCCTGATGGAGTCTCATGGCACAAGAAGATTAAAACAGTGTCTCCAATTTTAATAAAT TTTTGCAATCCATCAAAAAAAAAAAAAAAAAAtgttttgTGTTTTGGCTGGGTTTTTCCTTGTTCG CACCGGACACCTCCAGTGACCAGACGGCAAGGTTTTTATCCCAGTGTATATTaaaaagcggtcaggca gctaaaccaaaaggtttagcaattgcctctgatgagtcgctgaaatgcgacgaaaaccg (SEQ ID NO: 32). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 32. In some embodiments, the trans-splicing molecule denoted P1779 can comprise a sequence encoded by SEQ ID NO: 32. The trans-splicing molecule denoted P1779 may be transcribed into an RNA molecule.

The trans-splicing molecule denoted P1791 can comprise a sequence (e.g., DNA or RNA sequence) derived from the lncRNA GAS5 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATACGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggotcttctcagagccacccaggtctggg aaggagtgctgtctgcgcctgccctccgctgggcacagcgtggctgcttcaggggctaagcacacactttctgtcttctaaagggccgccacatgccag gagctcaggtgtgagcccggctctggctcttacctcatagggtcactcataggggcacagggagcagaacattgtacacagcgaggcaccaccoggct tggcatctgcctcggtggacttactacctctagaaggaaafacctgagttcctctggcctcagcfcctagagtgactggfgtgctgtccctgttactcttctgt caaggtgacaactgtgtgacccatcatctgtgtgtcaaagcaaggccctgcctgggcctctgctcctgtgctgaccccaaaggcaaatgctttgctagtttc cttccagttaatttcacctatgaatagatgtgtgaaaactgttcaaagccatacctgcacatgtttgaacttcaaaccctgtgggtgattcagtggcatctttctc taacccccagcctcccttcccacagaggccaccgtcatggccagttgctgcagtttctttccagagaacctgtgtatgtgtaaagctgtacaggcgtgggt acaccacacagcctgtcttgcactgtggactgttgagttactagtacatctaggtaagcaccgcatatctgtattcatgtctgccttggtcttttcaacatctgtg tggtagccgtgtttgaattacccattccctttttggggaaccattaagttgtttcagcaatttttactgtagataaggctataccgcatatctgtgtacatgggtttt tatgtacatgggcaagtatatctgtgagagaaaagtttcctcaggaggaattctgggcacagcatgtgtaaatttctaaatatgatggacacccccagcttcc acctcaaggaggttggtcccattgacattfccccacaccttcacccaggctgtgcccttaaacttggftatttgtcaatgtgagaagtggaaaatagtatttaat tgtagtttggatttgtatttotattggtgttttgTGTTTTGGCTGGGTTTTTCCTTGTTCGCACCGGACACCTCCAGTG ACCAGACGGCAAGGTTTTTATCCCAGTGTATATTaaaaagcggtcaggcagctaaaccaaaaggtttagcaattgcctct gatgagtcgctgaaatgcgacgaaaaccg (SEQ ID NO: 33). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 33. In some embodiments, the trans-splicing molecule denoted P1779 can comprise a sequence encoded by SEQ ID NO: 33. The trans-splicing molecule denoted P1779 may be transcribed into an RNA molecule.

The trans-splicing molecule denoted P1792 can comprise a sequence (e.g., DNA or RNA sequence) derived from the lncRNA NEAT1 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CCCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTGGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTGCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTACCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgaoggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaggttgtata cttactgattaataataagagctctttacatattaaggaaattaacccttttcaaatacattcctatttctcactaatctttaagttttattgtaatattttgctctttagttt atatatatatgtatatatatatatatgtatatatatatatatacatatatatatacatatatatatactaattttottttatggttoctggattttgtgagtagtttgaaaag gctaatccagctgaagattttgttgttgttgttaaaccccatgttttctcctaactctttttatttttattttggaggactctatctagacttaattttagcataacaagt gcttagttagcctgttgtccttacaccattttctggctaatacagctattaactattgatctgtctattcacgtgccagttcctaatggttttacatagtgta gacagggttagttagcctgttgtccttacaccattttctggctaatacgctattaactattgatctgtctattcacgtgccagttcctaatggttttacatagtgta atctgcacttcaaaatagcgaagggaagccctacctcattattctacttttccagaattctcctggctattccaggctgcatgtttaccttaaccttccctgtgat gtcttcatgccgttgtcttcttatgcaagaataaggtacgtctttccatccactcacgtctatttaatttgactttgcattacacagaaagctggtcttggtctgtct acctcggcatctagttgtcctcactgccccctagccgaccccaccccatctgactgactaccccatcacagagtacttttatttacgttftgctctgcctaatgg ttacttgatactgtcacgccgacagtgtccagttcagtggtctttgcagttgaaatgctcccgtacacactgtcttgttaaaaatgccagtaagttcatacaaac ccagcttgcacccaaggtcacattcagagagcgtagggctgggatgggttgttttccaagcttctgccactgtgtggctagctcttcccactgggaagttct gtgtacccggaatgtcggagtggagtcctgttctagtgtccagcacctgaccctgtgcccaacccctcaacagcctattcctgctgtgttttgTGTTTT GGCTGGGTTTTTCCTTGTTCGCACCGGACACCTCCAGTGACCAGACGGCAAGGTTTTTATCCC AGTGTATATTaaaaagcggtcaggcagctaaaccaaaaggfttagcaattgcctctgatgagtogctgaaatgcgacgaaaaccg

The trans-splicing molecule denoted P1793 can comprise a sequence (e.g., a DNA or RNA sequence) derived from the lncRNA NEAT1 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGGGGCAGACGT AGTACTTATTAGGAATGACTTGCTCGACGTAGTGGCATCAATCCATCTTTCAAAGCGGACTG TCAGAAGGATACGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaccacagcc tgctggaactttttacaaaatatgttgccatgctggaccctgggcactggacataagccccctggcagcctttttcatgtcacccaaaggggtaattgtccta ctggtggtctgtaagatgagttagggtgacttgctaatagacattgtaaatcttaatatttatgtatgtattttattattaccggttttccatttatgatggtaatattgt ttcttctaagaatatttatttttccttctaaafattgagataaaattcatgcttttgaaatgttctattcagtggctfttagtatatftgctatgttgtgcaaccatcgaca ctatccatttctagaactttttcgtcatcccaaacagacgctctgtattcataaaaaaataacttcctacctgtctctccccctagtctttggtaacctttgttatact ggtaaactttgltgtgctctctgtctgtgtgaatttgcctattctaggggcctcatataagtgtaatcatacagtatttgtctttttgggtctgtctgatttcacttagc gggttttcagggttcattcatgttgcagcatataacagtactgcgttcctttttctggctgaataatattccactgtatggatagaccccattttgtttattcacaca tcatttggacatttggattatttctggtttttggctattatgaacaatggtgctafgaacagttgcgfacaagtttttgtgtgaacatatgttttcaattctctcattata tacctaggagtagaattactgggtcatatggtaactgtatatttttgaggaactgccaaactattttcccacgtccatgcaccatttcacaftcccaccagtaag taagagggttccaatttctgcgcattcttgccaacactagttattatctgactttctggttataatcattctaatgagtgtgaagtagcctctggtgtcatttggattt gcatttctctgatgagtgatgctatcaagcacctttgctggtgctgttggccatatgtgtatgttccctggagaagtgtcttgttttgTGTTTTGGCTG GGTTTTTCCTTGTTCGCACCGGACACCTCCAGTGACCAGACGGCAAGGTTTTTATCCCAGTGT ATATTaaaaagcggtcaggcagctaaaccaaaaggtttagcaattgcctctgatgagtcgctgaaatgcgacgaaaaccg (SEQ ID NO: 34). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 34. In some embodiments, the trans-splicing molecule denoted P1779 can comprise a sequence encoded by SEQ ID NO: 34. The trans-splicing molecule denoted P1779 may be transcribed into an RNA molecule.

The trans-splicing molecule denoted P1794 can comprise a sequence (e.g., DNA or RNA sequence) derived from the lncRNA NEAT1 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggaogtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaAGCCC CTAGCGCAGACGGCGGAGAGCAGAGAGGGAGCGCGCCTTGGCTCGCTGGCCTTGGCGGCGG CTCCTCAGGAGAGCTGGGGCGCCCACGAGAGGATCCCTCACCCGGGTCTCTCCTCAGGGATG ACATCATCCGTCCACCTCCTTGTCTTCAAGGACCACCTCCTCTCCATGCTGAGCTGCTGCCAA GGGGCCTGCTGCCCATCTACACCTCACGAGGGCACTAGGAGCACGGTTTCCTGGATCCCACC AACATACAAAGCAGCCACTCACTGACCCCCAGGACCAGGATGGCAAAGGATGAAGAGGACC GGAACTGACCAGCCAGCTGTCCCTCTTACCTAAAGACTTAAACCAATGCCCTAGTGAGGGGG CATTGGGCATTAAGCCCTGACCTTTGCTATGCTCATACTTTGACTCTATGAGTACTTTCCTAT AAGTCTTTGCTTGTGTTCACCTGCTAGCAAACTGGAGTGTTTCCCTCCCCAAGGGGGTGTCAG TCTTTGTCGACTGACTCTGTCATCACCCTTATGATGTCCTGAATGGAAGGATCCCTTTGGGAA ATTCTCAGGAGGGGGACCTGGGCCAAGGGCTTGGCCAGCATCCTGCTGGCAACTCCAAGGC CCTGGGTGGGCTTCTGGAATGAGCATGCTACTGAATCACCAAAGGCACGCCCGACCTCTCTG AAGATCTTCCTATCCTTTTCTGGGGGAATGGGGTCGATGAGAGCAACCTCCTAGGGTTGTTG TGAGAATTAAATGAGATAAAAGAGGCCTCAGGCAGGATCTGGCATAGtgttttgTGTTTTGGCTG GGTTTTTCCTTGTTCGCACCGGACACCTCCAGTGACCAGACGGCAAGGTTTTTATCCCAGTGT ATATTaaaaagcggtcaggcagctaaaccaaaaggtttagcaattgcctctgatgagtcgctgaaatgcgacgaaaaccg (SEQ ID NO: 35). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 35. In some embodiments, the trans-splicing molecule denoted P1779 can comprise a sequence encoded by SEQ ID NO: 35. The trans-splicing molecule denoted P1779 may be transcribed into an RNA molecule.

The trans-splicing molecule denoted P1795 can comprise a sequence (e.g., DNA or RNA sequence) derived from the lncRNA MEG3 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA CCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgoggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaGAGGA GGTGATCAGCAAATGTTTGTTGAAAAGGTTTGACAGGTCAGTCCCTTCCCACCCCTCTTGCTT GTCTTACTTGTCTTATTTATTCTCCAACAGCACTCCAGGCAGCCCTTGTCCACGGGCTCTCCT TGCATCAGCCAAGCTTCTTGAAAGGCCTGTCTACACTTGCTGTCTTCCTTCCTCACCTCCAAT TTCCTCTTCAACCCACTGCTTCCTGACTCGCTCTACTCCGTGGAAGCACGCTCACAAAGGCAC GTGGGCCGTGGCCCGGCTGGGTCGGCTGAAGAACTGCGGATGGAAGCTGCGGAAGAGGCCC TGATGGGGCCCACCATCCCGGACCCAAGTCTTCTTCCTGGGGGGCCTCTCGTCTCCTTCCTGG TTTGGGCGGAAGCCATCACCTGGATGCCTACGTGGGAAGGGACCTCGAATGTGGGACCCCA GCCCCTCTCCAGCTCGAAATCCCTCCACAGCCACGGGGACACCCTGCACCTATTCCCACGGG ACAGGCTGGACCCAGAGACTCTGGACCCGGGGCCTCCCCTTGAGTAGAGACCCGCCCTCTGA CTGATGGACGCCGCTGACCTGGGGTCAGACCCGTGGGCTGGACCCCTGCCCACCCCGCAGG AACCCTGAGGCCTAGGGGAGCTGTTGAGCCTTCAGTGTCTGCATGTGGGAAGTGGGCTCCTT CACCTACCTCACAGGGCTGTTGTGAGGGGCGCTGTGATGCGGTTCCAAAGCACAGGGCTTGG CGCACCCCACTGTGCTCTCAATAAATGTGTTTCCTGTCTTAACAAAtgttttgTGTTTTGGCTGGG TTTTTCCTTGTTCGCACCGGACACCTCCAGTGACCAGACGGCAAGGTTTTTATCCCAGTGTAT ATTaaaaagcggtcaggcagctaaaccaaaaggtttagcaattgcctctgatgagtcgctgaaatgcgacgaaaaccg (SEQ ID NO: 36). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 36. In some embodiments, the trans-splicing molecule denoted P1779 can comprise a sequence encoded by SEQ ID NO: 36. The trans-splicing molecule denoted P1779 may be transcribed into an RNA molecule.

The trans-splicing molecule denoted P1796 can comprise a sequence (e.g., DNA or RNA sequence) derived from the lncRNA MEG2 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTGGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctCCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaTTGGC CCCACTGTGCACCACACACTCCTTTCCCAGCCCAGGGGCACGCGAACAAAATGAGGCTCAA GCTGACCAGGCCGAGCCGGAGGAACGCTGGGGCTTGGCAGCAGAAGGGATGGGACCAGAG AGAAGGGTGTGGAGGAGACCCCAGTGAGGGCCAGGACATTTCAGGTAAAGAGAGGTCATAT CTCCGTACCTCACTTCCTGACACAAACAAGTTTTCACTGTTGTCAGCAACAAAGCCCTAATAT AGCTGCGGAAGAGAAAAACTGCATTGCATTTTGCCTCCTGCAAGCATCATCAACAGTTACTG GAGGAACGTAATTCCAGAAAGCTTGAAAGCCGTGGTGATGGTAATTATGTATCAAATGCCTG GTTCTATTTCTGTTATTATTGTTTTGTCATTTCTGTTTTCCCAGCGATCTGACTGAACTCGCAG AGGGACAAATCCAGTTTTTCTTTTTGACTTTTGTCAAACTAAATCAGGCCTGATAGAAAACTC ATTGCTCTCCGGGGAAACAAAGTAGGAGCCACGAAATGTCATTTTAACAGAGCGTGGGTTTG GTGACTGTAGGAAAGGATTTGAGGACGCTCCTTCTGTTCGGCTTCCTATGTCATGAGCACAG GCTCCACGCACGCACAGACACCACGGCTCCCGGATGCTGTGGCTCCCCGATCGGGGCTCCTG CAGCGCCAGAAGCCCCTCCGGGATGCTTCGAGGGGCTCCCGGTGGGTGGAGGTACGGACGC CGCTGCGGCCGCCGCCGCCAGTCCTGCTGCTGTTGTTGCTGCTGCAGTCACGTGGGAGCCCC TTTAAGTTTCCATAGAGAGGCCTCTCTGGTGTCACATGATGGACATGATATAATGAAACAAC ATTGTGGAGAGGAAAGCATTAGGGGAGCCCACGGCTACAAAAACAAGTGAGTGAGAAGAG GTGGGAGGAAGAGAAACTACGCCACCTCCCCTGCAGCCGAGTGCACGCAGCAGCCTGGCGT GACAAGTGGGCGACGCCGGGGGGCAGGGAGCCGGGGTCCTTGGCCCTGGCCGGGGtgttttgTG TTTTGGCTGGGTTTTTCCTTGTTCGCACCGGACACCTCCAGTGACCAGACGGCAAGGTTTTTA TCCCAGTGTATATTaaaaagcggtcaggcagctaaaccaaaaggtttagcaattgcctotgatgagtcgctgaaatgcgacgaaaaccg (SEQ ID NO: 37). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 37. In some embodiments, the trans-splicing molecule denoted P1779 can comprise a sequence encoded by SEQ ID NO: 37. The trans-splicing molecule denoted P1779 may be transcribed into an RNA molecule.

The trans-splicing molecule denoted P1797 can comprise a sequence (e.g., DNA or RNA sequence) derived from the lncRNA PINT along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGLAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaAAATG AAGGAGTTGAGAAGTACATACTCTGAGGTTTATCCTAAATAAACTGAAGTCTAGGCAAGTG GCTGAGCTAGGTTTGCCATGAATCAATCTGCTGAGTATATTTCTTGGTAACTAGTTCATCTTT CCTTAATTCAGTCAACAAATATGATATCAAGTTTAGTAAGATGATGACAGTTGTCATGTAAT ACTCCATCTGGAAGATTGTATGCTTCTCAGCTTCCCTAACTTTTCTTAATCTACCTTAAGCTTC CTTTCTGAAAGGTTTCTGTCTTTCCTAACAGTCTCTTCTTTCCGCCAGCTACCTCTTGTGTTTA AGAATTAAAGGAAGATGAGCTAGGAATCAAGAAATACGCAGAACTGATTCCTGTCACTGGC GCCACCCTGCCACTTGCCCAGAGCTGTGGTATAAATTTTTTTAATAGGCTGAAATAAAAATA TGAATAGCATTTTGGTATTAAGCATTAAATTGATAAAGGCTATGAGATACACCTGCTCTCAA CCATGTTAATTTTTTATTATTGGTATTAATAAATTATTACTTACTAACATATTAACAAGAATT GCATTGAGAAACAAAGCATCCACAGGCCAAATCTTCTGAATTTCAAATGTTTATATTAATGC GTTGTATTCTAGAAAAGTAGAATTGTCTTAAGTAGCTTTGTAATATAAAGTCATCTATCAGCC CATTACACCTATTAGAATGTTTTAACCTTTATTTCCCACTTTTTTGTTTCTAGACTGAGTGTAC TTGGTCTGGTATTTGTGGCATAACTTACGGAACACATAAGAATGATACAGATACTATTTAAT GATGACCTAATACAAGCTTAGATAGCTAAGGTGAAAGCTTCTATGGCCTTAACATTTTCCTC TTGAAGAATGTATTTTCTGTAATAAAATACAGTGGCTACTTGAAATCTATAAACTTATGTGA GGTCTGGATAAATCTGAGCAACTTTCTTCTTTGTGCTCCAGGAACCTACGCACTATATATATA AATAAAGCTTAAGTAAACATCACTGCAAAAAAAAAAAAAAAAAtgttttgTGTTTTGGCTGGGTT TTTCCTTGTTCGCACCGGACACCTCCAGTGACCAGACGGCAAGGTTTTTATCCCAGTGTATAT Taaaaagcggtcaggcagctaaaccaaaaggtttagcaattgcctctgatgagtcgctgaaatgcgacgaaaaccg (SEQ ID NO: 38). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 38. In some embodiments, the trans-splicing molecule denoted P1779 can comprise a sequence encoded by SEQ ID NO: 38. The trans-splicing molecule denoted P1779 may be transcribed into an RNA molecule.

The trans-splicing molecule denoted P1798 can comprise a sequence (e.g., DNA or RNA sequence) derived from the lncRNA PINT along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATACGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaACAAT CCAATTCCTAGATGCTTTCTCCAGTTATGGCCCTGACTGGGATCTCCGAAGAATGACTCAGCT GAAACTATTTCATCATTGCCTACATTTGTCTTCAGACGACCTGCAGTACAAACTTGGCTTAAA CCTGCAGTTGACAATGGATTCACCGTGGGAGAATAAATTACAATGGATAAAAAACAGCATT ACAATCCCTCAATAAAATGGGATTTTAACAGACAAAGCTCCTGGTATGTGGTCGTTCTCAGG CCCAAAGGCCCATTTTGCTTTTGGCTGCAACTCTGTAACTCTGTAACAGAGACCAAGTGCCT GCAGGGAGCAGATGTTTGGGGCAGTATCTGCTAAGGTGGCTTTGCTGAAACTTGTTTCTCTC ATTGGGTCTTGGAAGTGGCGAATAGGACGTTCCTATAGAAAAACTCAGAAAGAGACTGTGA AAGATAATCTTAAAGAGGCACTCACGAAACACAAGCTCCTTCTCCGGGAAAAAGAATCATC ACCCTTGTTATCCCATGAAAATTCATCTTCTCCTTTTCAGCTGATATGAGGACTTGATCTGTC CTTTCCTATAAGTGCCATCTGTGTTTAAATGAGGCTGTTACTCTAACTCAAAGCAGACTTGCA AGAGTAAAATAATCAATCTTCATCATAtgttttgTGTTTTGGCTGGGTTTTTCCTTGTTCGCACCG CACACCTCCAGTGACCAGACGGCAAGGTTTTTATCCCAGTGTATATTaaaaagcggtcaggcagctaaacc aaaaggtttagcaattgcctctgatgagtcgctgaaatgcgacgaaaaccg (SEQ ID NO: 39). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 39. In some embodiments, the trans-splicing molecule denoted P1779 can comprise a sequence encoded by SEQ ID NO: 39. The trans-splicing molecule denoted P1779 may be transcribed into an RNA molecule.

The trans-splicing molecule denoted P1799 can comprise a sequence (e.g., DNA or RNA sequence) derived from the lncRNA XLOC_003526 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CCCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTGGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCcCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggg agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaAGAGT GAAGGAGCAAAGGGGACTCGGCCGCCATGTTAGGAGTACTGGGACGATTCCGCGGAGCCGG GCAGAGGTTTTAGGGGAATGATTAACAAAGGCGTCCGAAGAAATCGTTGTTGGAAGGTGAC CAAGGTGGAAAGAGACGTTGCTTTGGCCCTGCAAGTAAGAAGAGAGAGGGAATAGCCTGAA GGAGTAACACTAAATTTAAAATGACACTTTTTTACCAACCAGCGAAAGCAGATGTTCAAAGG GGATATTGGCCAGAGTCTGTCACACTAAGATGAGAAATGTCCTTTCTTCCTGAAGGTGTCTG ATGTGTAAAAATATGATATACTTTGTGCTGTTTCCTCCCTTCCCTTTTGCATATTATTCTGAAA CAACATTAACTAGTTACTTTGCGTCATTGAAGGTATGCACTTCCCCTCTATGTTAGGAGTGAA TAAAATTAAAAATAGATCCTTATAACAAAGAAAGGCAGATAGAATGATTAAAAATGACCAA AACATGTTAGAAACAGTCTCTCAGGTGTATGCAGATGGTAATTACAAAAATACTTTTTCAAA AAATGATCTTCTGTGTCATGTTTCTGGGAACAAGTCAAGATGAATGAGTTTGATTTTTAAGC AGAAGTAGTATGTGTTGGTGTCATCCATGAATACAACAATGAAAAAGGCtgttttgTGTTTTGGC TGGGTTTTTCCTTGTTCGCACCGGACACCTCCAGTGACCAGACGGCAAGGTTTTTATCCCAGT GTATATTaaaaagcggtcaggcagctaaaccaaaaggtttagcaattgcctctgatgagtcgctgaaatgcgacgaaaaccg (SEQ ID NO: 40). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 40. In some embodiments, the trans-splicing molecule denoted P1779 can comprise a sequence encoded by SEQ ID NO: 40. The trans-splicing molecule denoted P1779 may be transcribed into an RNA molecule.

The trans-splicing molecule denoted P1800 can comprise a sequence (e.g., DNA or RNA sequence) derived from the lncRNA XLOC_009233 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGtGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGGGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgoggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAccaaaggctcttctcagagccacccaCCCTT CTCCAGAGCGCCGTCGACCCCACGCCACGCAGAGGTGAAGATACGCAGGACGTCCTCTAAA GCAACTTCCGTGGGTGCCAGGTTGTGGGCTGGGGGGGGTGCTGACCTTGAGAGAGCGGAGG AGGCGCAGGCGCAGCACAGTTTAAACTTGATTTTGTGAGGGGACGAACAGCGTGACAATTA AAAGAAGATCTGGGAATCATCCAATGTGATAATTTATGTGAAATCTTTCTGCAAACTATACA GTATGATAAAAATATAAGGTAGTTTCACTGGAAACAACAGAAGACTAGAAGCTGATGTGAC GTGGCAATATCAACCAGGAATACCCATCTCGAGTTCTGGATATGTGCTCTGAGAAGGTGCCC ATGTCACCGACCACACTGGAGGCCAATGCAGATACTGGGGGAAGGTTCCATGGTAGCTAAG TGTGGACAAGCTAATCACTGAAGTTCCCTGATGCAGAGTTGATAATCTCTCTAGAGTTGGAT GAAAGACTGTGCTGCCTTGAAGCTCTGAGAGATGCCATGCCAATTCAAGGCAGGGTCTTTAT TGTTGAAACTCTTGCGTAATGACATCTTTATTCAGGTGAAAATACAGGATGAATTTCAACTA TATGATATTGTTTATGTTCCTCAGACATGTTATTTGTCTTTACAAAGATTGGTTTCAATAAAC TGTGTGACTGATATTAAATAAACATGGAATTTTACACATTCATAATAAAAAAAAAAtgttttgTG TTTTGGCTGGGTTTTTCCTTGTTCGCACCGGACACCTCCAGTGACCAGACGGCAAGGTTTTTA TCCCAGTGTATATTaaaaagcggtcaggcagctaaaccaaaaggtttagcaattgcctctgatgagtcgctgaaatgcgacgaaaaccg (SEQ ID NO: 41). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 41. In some embodiments, the trans-splicing molecule denoted P1779 can comprise a sequence encoded by SEQ ID NO: 41. The trans-splicing molecule denoted P1779 may be transcribed into an RNA molecule.

The trans-splicing molecule denoted P1801 can comprise a sequence (e.g., DNA or RNA sequence) derived from the lncRNA XLOC_004456 along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATGCCAATTGGAAGGAGTTCAACAAATT AGCGTGAGCCTCGCGGAGGGGACAGCCACCGTACTCTATAACCCCGCCGTAATTTCCCCTGA AGAGCTGCGCGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTGCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGTGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCcCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTA (SEQ ID NO: 42). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 42. In some embodiments, the trans-splicing molecule denoted P1779 can comprise a sequence encoded by SEQ ID NO: 42. The trans-splicing molecule denoted P1779 may be transcribed into an RNA molecule.

The trans-splicing molecule denoted P1802 can comprise a sequence (e.g., DNA or RNA sequence) derived from the lncRNA along with the full length coding sequence of the ATP7B gene, an antisense region that targets ALB, a splicing domain and a 3′ terminal KSHV sequence and hammerhead ribozyme: gccaccATGCCTGAGCAGGAGAGACAGATCACCGCACGtgAAGGAGCTTCTCGAAAGATCCTGA GCAAACTTAGCCTGCCTACTAGGGCTTGGGAACCCGCTATGAAGAAGAGTTTTGCATTTGAT AACGTCGGATACGAAGGAGGACTCGATGGTCTGGGACCGAGTTCACAAGTGGCTACCTCAA CTGTACGGATTTTGGGTATGACCTGCCAAAGCTGCGTTAAGAGCATTGAGGATCGGATATCC AATCTTAAAGGTATTATTAGCATGAAAGTTTCTTTGGAACAGGGCTCAGCGACTGTTAAATA CGTCCCGAGTGTTGTTTGTCTTCAGCAGGTCTGTCACCAGATAGGCGATATGGGCTTCGAAG CGTCCATAGCTGAAGGGAAAGCGGCATCTTGGCCGAGCAGGTCTCTTCCCGCCCAAGAAGC CGTCGTCAAGCTTCGGGTTGAGGGTATGACATGTCAGTCCTGCGTGAGCTCCATAGAAGGTA AAGTTCGCAAGCTTCAGGGCGTGGTGCGGGTCAAGGTCTCCCTCAGTAATCAAGAGGCCGTC ATAACATACCAGCCATATCTTATTCAACCAGAGGACCTGAGGGATCATGTAAACGACATGG GCTTTGAAGCGGCAATCAAGAGTAAGGTCGCGCCTCTTTCCCTTGGCCCAATAGACATTGAG CGCCTCCAAAGTACGAACCCGAAAAGGCCGTTGAGTTCTGCCAACCAAAACTTTAATAATAG CGAGACTCTTGGCCACCAAGGGTCTCACGTAGTTACGTTGCAGCTCCGAATCGATGGTATGC ACTGCAAGTCCTGTGTCCTGAATATTGAGGAGAATATAGGTCAACTTCTTGGTGTACAGTCT ATCCAGGTAAGTCTTGAAAATAAAACGGCCCAGGTTAAGTACGACCCCTCATGTACTTCACC GGTGGCACTTCAAAGAGCAATAGAAGCCCTGCCGCCAGGGAATTTTAAAGTCTCTTTGCCTG ATGGGGCTGAAGGCTCTGGTACAGATCACAGGAGCTCTTCTAGCCATAGCCCGGGTTCCCCG CCCCGAAATCAGGTGCAAGGAACATGTAGCACGACCCTCATCGCAATTGCGGGTATGACTTG TGCGAGTTGTGTACATAGCATAGAGGGAATGATATCCCAATTGGAAGGAGTTCAACAAATT AGCGTGAGCCTCGCGGAGGGGACAGCCACCGTACTCTATAACCCCGCCGTAATTTCCCCTGA AGAGCTGCGGGCCGCGATAGAGGATATGGGATTCGAAGCCTCTGTAGTATCAGAAAGTTGT TCCACGAACCCACTCGGGAACCATTCAGCTGGCAATTCTATGGTACAGACAACCGATGGAAC ACCAACCAGTCTTCAAGAAGTTGCTCCTCACACTGGACGACTCCCCGCGAATCATGCTCCTG ATATCTTGGCTAAGAGCCCCCAATCAACACGGGCCGTCGCTCCACAAAAGTGTTTTCTTCAA ATCAAGGGCATGACCTGTGCATCATGCGTTTCCAATATAGAAAGGAATTTGCAGAAAGAGG CCGGGGTACTTTCTGTACTCGTTGCTCTCATGGCTGGAAAAGCTGAAATTAAATACGACCCA GAAGTCATACAGCCTTTGGAAATTGCCCAATTCATACAAGATCTCGGATTCGAAGCTGCCGT AATGGAGGACTATGCCGGCAGCGATGGTAATATCGAGTTGACCATTACCGGAATGACATGT GCTAGCTGCGTACATAATATCGAGTCCAAGCTTACGAGAACGAACGGAATCACATACGCTTC AGTAGCTCTCGCCACAAGTAAGGCCCTGGTAAAATTTGACCCAGAGATTATTGGACCTAGAG ACATCATTAAAATCATCGAAGAAATCGGTTTTCATGCGAGCCTCGCGCAGCGCAACCCGAAT GCGCACCATCTGGATCATAAGATGGAAATAAAGCAATGGAAAAAATCATTCCTTTGCAGCTT GGTATTCGGTATCCCCGTCATGGCTTTGATGATCTACATGCTGATCCCGTCAAACGAGCCAC ATCAGAGCATGGTACTCGATCATAACATTATACCGGGTCTTAGCATACTTAATCTTATTTTTT TTATCCTTTGCACATTCGTCCAACTTTTGGGGGGTTGGTACTTCTACGTGCAGGCGTATAAAT CTCTGCGGCATCGCAGCGCCAATATGGATGTTTTGATCGTATTGGCTACTAGCATTGCTTATG TATATTCCCTGGTGATCCTCGTTGTGGCGGTGGCGGAAAAAGCAGAGCGGTCACCAGTCACG TTCTTTGATACGCCACCAATGTTGTTCGTGTTCATTGCGCTCGGAAGGTGGTTGGAGCACCTT GCAAAATCTAAAACAAGTGAAGCCCTGGCTAAACTTATGTCACTTCAGGCCACAGAGGCCA CCGTTGTTACCCTCGGTGAAGACAACCTTATCATTAGGGAAGAACAAGTTCCCATGGAACTC GTTCAGCGAGGAGATATAGTTAGGGTGGTCCCTGGGGGTAAATTTCCCGTCGACGGGAAGG TTCTTGAGGGAAACACGATGGCTGACGAAAGCCTCATAACTGGGGAAGCGATGCCGGTGAC CAAAAAACCTGGGAGTACAGTGATAGCGGGCTCTATTAACGCACATGGTTCTGTGCTTATTA AGGCTACACACGTAGGTAACGACACCACCTTGGCACAGATCGTTAAACTTGTTGAGGAGGC CCAAATGTCTAAAGCACCTATACAACAACTCGCGGACCGGTTTTCAGGATACTTTGTACCCT TTATTATAATAATGAGCACACTGACTCTTGTGGTATGGATTGTTATCGGCTTTATCGATTTCG GAGTCGTACAAAAATATTTCCCGAACCCAAATAAGCACATCAGTCAGACGGAAGTCATCAT AAGGTTTGCTTTTCAAACCAGCATTACAGTCCTCTGCATAGCATGCCCGTGTTCTCTCGGCCT GGCCACACCCACAGCGGTCATGGTAGGCACCGGTGTTGCTGCCCAGAACGGTATACTTATCA AAGGTGGCAAGCCGTTGGAAATGGCTCATAAGATCAAAACGGTGATGTTCGATAAGACTGG AACTATCACCCATGGGGTCCCTCGGTTATGCGAGTTCTGTTGCTGGGCGATGTTGCCACTCT GCCACTCAGAAAAGTTCTCGCCGTAGTCGGAACTGCCGAAGCGTCAAGCGAACACCCCCTG GGTGTAGCAGTCACTAAATACTGTAAGGAGGAGCTTGGCACAGAGACGCTGGGTTACTGTA CAGACTTCCAAGCCGTTCCTGGGTGCGGCATCGGATGTAAGGTGTCAAATGTAGAGGGTATC CTGGCTCACTCCGAACGACCCCTTAGTGCGCCGGCCTCCCATCTTAATGAAGCCGGTTCTTTG CCTGCTGAGAAAGACGCAGCCCCTCAGACCTTTTCCGTTCTCATAGGCAATCGGGAATGGTT GCGGAGAAACGGCTTGACTATTAGCAGTGATGTGTCCGATGCAATGACGGATCATGAGATG AAAGGTCAAACCGCGATTCTCGTGGCTATCGATGGAGTGCTTTGTGGAATGATAGCAATCGC AGACGCCGTCAAACAAGAGGCAGCCCTCGCCGTACACACACTGCAATCCATGGGCGTGGAC GTCGTTTTGATCACGGGCGATAATCGGAAAACGGCCAGGGCCATTGCAACTCAAGTAGGGA TAAACAAGGTGTTTGCCGAAGTTCTGCCCAGTCATAAAGTAGCTAAAGTTCAGGAACTTCAG AACAAGGGGAAGAAAGTAGCCATGGTGGGGGATGGCGTCAACGACTCTCCAGCCCTTGCCC AGGCTGACATGGGCGTCGCTATAGGGACCGGGACCGACGTCGCAATCGAGGCGGCAGACGT AGTACTTATTAGGAATGACTTGCTCGACGTAGTGGCATCAATCCATCTTTCAAAGCGGACTG TCAGAAGGATaCGGATCAACTTGGTACTGGCGCTGATATACAACCTCGTAGGGATACCTATA GCTGCAGGCGTCTTCATGCCTATCGGCATTGTTCTGCAGCCATGGATGGGATCAGCTGCGAT GGCTGCGAGTAGCGTATCCGTAGTGCTGTCCTCTCTGCAACTTAAGTGCTATAAGAAACCAG ATCTTGAAAGGTACGAGGCGCAGGCACACGGGCACATGAAACCCTTGACCGCTTCTCAAGTT AGTGTACACATCGGTATGGATGATCGCTGGCGGGATTCCCCTCGGGCTACTCCGTGGGATCA AGTCAGTTATGTGTCTCAAGTATCACTTTCTTCACTTACCTCTGACAAGCCCAGCCGCCACagc gctGCAGCCGACGACGATGGGGACAAGTGGAGTCTCCTGTTGAATGGTCGAGACGAAGAACA ATATATCgactacaaagaccatgacggtgattataaagatcatgacatcgactataaggatgacgatgacaaaggctccggcgagggcagggga agtcttctaacatgcggggacgtggaggaaaatcccggcccatcATCTTCTTCGAGGATGACGGCAACTACAAGTCGC GCGCCGAGGTAAGAGAGCTCGTTGCGATATTATTACAGCAACGAAAACTGCAACGGACCTC CCGGGGCtaatgcggccgcGGTAGATCTCATGTTAAGGGTTCTTACTATAATAAAATAAGATAAAT AAATAAATAAATACATGCTATCATATCCCTATTAGCCTCTACAAAAAGAAACCAAACCTCAA ATTTTAAAAAGAGGTGTTTATAAATGACAAATTGCCATTTAtgtttgTGTTTTGGCTGGGTTTTT CCTTGTTCGCACCGGACACCTCCAGTGACCAGACGGCAAGGTTTTTATCCCAGTGTATATTaaa aagcggtcaggcagctaaaccaaaaggtttagcaattgcctctgatgagtcgctgaaatgcgacgaaaaccg (SEQ ID NO: 43). In some embodiments, trans-splicing molecule denoted P1779 can comprise at least about 60%, about 65%, about 70%, about 75%, about 80%, about 85%, about 90%, about 95%, about 97.5%, about 98%, about 99%, or about 100% identity with a sequence encoded by SEQ ID NO: 43. In some embodiments, the trans-splicing molecule denoted P1779 can comprise a sequence encoded by SEQ ID NO: 43. The trans-splicing molecule denoted P1779 may be transcribed into an RNA molecule.

The present disclosure provides vectors comprising or encoding nucleic acids as described herein. In some embodiments of the compositions and methods of the present disclosure, a vector can comprise or encodes a nucleic acid of the present disclosure. The nucleic acid may comprise or encode a trans-splicing nucleic acid. In some embodiments, the vector can encode or can comprise a DNA sequence. In some embodiments, the vector can encode or can comprise an RNA sequence. In some embodiments, the vector can comprise or can encode at least one trans-splicing nucleic acid of the present disclosure. In some embodiments, the vector can comprise or can encode one or more trans-splicing nucleic acid(s) of the present disclosure. In some embodiments, the vector can comprise or can encode two or more trans-splicing nucleic acids of the present disclosure. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprise, a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex, an exosome or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods the present disclosure, a vector the present disclosure can be a viral vector. In some embodiments, the viral vector can comprise a sequence isolated or derived from a retrovirus. In some embodiments, the viral vector can comprise a sequence isolated or derived from a lentivirus. In some embodiments, the viral vector can comprise a sequence isolated or derived from an adenovirus. In some embodiments, the viral vector can comprise a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods the present disclosure, the viral vector can comprise a sequence isolated or derived from an adeno-associated virus (AAV). In some embodiments, the viral vector can comprise an inverted terminal repeat sequence or a capsid sequence that is isolated or derived from an AAV of serotype AAV1, AAV2, AAV3, AAV4, AAV5, AAV6, AAV7, AAV8, AAV9, AAV 10, AAV11 or AAV 12. In some embodiments, the viral vector is replication incompetent. In some embodiments, the viral vector is isolated or recombinant (rAAV). In some embodiments, the viral vector is self-complementary (scAAV).

In some embodiments of the compositions and methods the present disclosure, a vector the present disclosure can be a non-viral vector. In some embodiments, the vector can comprise or consists of a nanoparticle, a micelle, a liposome or lipoplex, a polymersome, a polyplex or a dendrimer. In some embodiments, the vector can be an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Non-limiting examples of expression control elements include but are not limited to promoters, enhancers, microRNAs, post-transcriptional regulatory elements, polyadenylation signal sequences, 5′ or 3′ untranslated regions, and introns.

Expression control elements may be constitutive, inducible, repressible, or tissue-specific, for example. A “promoter” is a control sequence that is a region of a polynucleotide sequence at which initiation and rate of transcription are controlled. It may comprise genetic elements at which regulatory proteins and molecules may bind such as RNA polymerase and other transcription factors. In some embodiments, expression control by a promoter is tissue-specific. Non-limiting examples of promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, H1 and U6 promoters. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

In some embodiments, the liposome, lipoplex, or nanoparticle can further comprise a non-cationic lipid, a PEG conjugated lipid, a sterol, or any combination thereof.

In some embodiments, the liposome, lipoplex, or nanoparticle further can comprise a non-cationic lipid, wherein the non-ionic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPO), dipalmitoylphosphatidylglycerol (DPPG), diceleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphospboethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanoktmine (SOPE), hydrogenated soy pbosphatidylcholine (HSPC), egg phosphatidyicholine (EPC), dioleoylphosphatidylserine (DOPS), sphingomyelin (SM), dimyristoyl phosphatidylcholine (DMPC), dimyristoyl phosphatidylglycerrol (DMPG), distearoylphosphatidylglyccerol (DSPG), dierucoylphosphatidylcholine (DEPC), palmitoylolcyolphosphatidylglycerol (POPO), dielaidoyl-phosphatidylethanolmnine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine and non-cationic.

In some embodiments, the liposome, lipoplex, or nanoparticle further can comprise a conjugated lipid, wherein the conjugated lipid, wherein the conjugated-lipid is selected from the group consisting of PEG-diacylglycerol (DAG) (such as 1-(monomethoxy-polyethyleneglycol)-2,3-dimyristoylglycerol (PEG-DMG)). PEG-dialkyloxypropyl (DAA), PEG-phospholipid, PEG-ceramide (Cer), a pegylated phosphatidylethanoloamine (PPG-PE), PEG succinate diacylglycerol (PEGS-DAG) (such as 4-O-(2′,3′-di(tetradecanoyloxy)propyl-1-0-(w-methoxy(polyethoxy)ethyl) butanedioate (PEG-S-DMG)). PEG dialkoxypropylcarbam, N-(carbonyl-methoxy poly ethylene glycol 2000)-1,2-distearoyl-sn-glycero-3-phosphoethanolamine sodium salt.

An “enhancer” is a region of DNA that can be bound by activating proteins to increase the likelihood or frequency of transcription. Non-limiting examples of enhancers and post-transcriptional regulatory elements include the CMV enhancer and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 0.3 kb to 4.75 kb. In some embodiments, non-limiting examples of AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh74 vector, a modified AAV.rh74 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. In some embodiments, non-limiting examples of lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (Fly) vector, a Visna/maedi virus (VNVVMV) vector, a modified d Visnaimaedi virus (VNV/VMV) vector, a caprinc arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments, the liposome, lipoplex, or nanoparticle further can comprise cholesterol or a cholesterol derivative.

In some embodiments, the liposome, lipoplex, or nanoparticle further can comprise an ionizable lipid, a non-cationic lipid, a conjugated lipid that inhibits aggregation of particles, and a sterol. The amount of the ionizable lipid, the non-cationic lipid, the conjugated lipid that inhibits aggregation of particles, and the sterol can be varied independently. In some embodiments, the lipid nanoparticle can comprise an ionizable lipid in an amount from about 20 mol % to about 90 mol % of the total lipid present in the particle, a non-cationic lipid in an amount from about 5 mol % to about 30 mol % of the total lipid present in the particle, a conjugated lipid that inhibits aggregation of particles in an amount from about 0.5 mol % to about 20 mol % of the total lipid present in the particle, and a sterol in an amount from about 20 mol % to about 50 mol % of the total lipid present in the particle.

The ratio of total lipid to DNA vector can be varied as desired. For example, the total lipid to DNA vector (mass or weight) ratio can be from about 10:1 to about 30:1.

In some embodiments of the compositions and methods the present disclosure, an expression vector, viral vector or non-viral vector provided herein, can include without limitation, an expression control element. An “expression control element” as used herein refers to any sequence that regulates the expression of a coding sequence, such as a gene. Non-limiting examples of expression control elements include promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may comprise genetic elements at which regulatory proteins and molecules may bind such as RNA polymerize and other transcription factors. In some embodiments, expression control by a promoter is tissue-specific. Non-limiting examples of promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An“enhancer” is a region of DNA that can be bound by activating proteins to increase the likelihood or frequency of transcription. Non-limiting examples of enhancers and posttranscriptional regulatory elements include the CMV enhancer and WPRE.

In some embodiments of the compositions and methods of the present disclosure, an expression vector, viral vector or non-viral vector provided herein, can include without limitation, an expression control element. An “expression control element” as used herein refers to any sequence that regulates the expression of a coding sequence, such as a gene. Examples of expression control elements include, but are not limited to, promoters, enhancers, microRNAs, post-transcriptional regulatory elements, polyadenylation signal sequences, 5′ or 3′ untranslated regions, and introns.

In some embodiments of the compositions and methods the present disclosure, an expression vector, viral vector or non-viral vector provided herein, can include without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments, the vector can be a viral vector. In some embodiments, the vector can be an adenoviral vector, an adeno-associated viral (AAV) vector, or a lentiviral vector. In some embodiments, the vector can be a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further can comprise one or more expression control elements operably linked to the polynucleotide. In some embodiments, the vector further can comprise one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 0.3 kb to 4.75 kb. In some embodiments, non-limiting examples of AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modifier AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh74 vector, a modified AAV.rh74 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Any lentiviral vectors may be used with the methods and compositions as disclosed herein (see, e.g., Trono D. (2002) Lentiviral vectors, New York: Spring-Verlag Berlin Heidelberg and Durand et al. (2011) Viruses 3(2):132-159 doi: 10.3390/v3020132, which is incorporated herein by reference in its entirety). In some embodiments, non-limiting examples of lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (EIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Cells and Tissues

Compositions and method as disclosed herein can be administered to a cell or tissue. The nucleic acids provided herein can enable replacement of arbitrary, missing, or incorrect sequences in a target RNA molecule. The target RNA molecule may be in a cell, a tissue, an organ, or in an organism. The cell, tissue, or organ may be provided in vitro or in vivo. In some embodiments, DNA molecules provided herein can enable replacement of arbitrary, missing, or incorrect sequences in RNA molecules of living cells. In some instances, the DNA molecule can comprise a replacement sequence that can be trans-spliced into RNA in order to modify (e.g., fix) the sequence. In some instances, modification or fixing of the RNA via trans-splicing can increase or decrease protein production. In some embodiments, the nucleic acids provided herein can enable localization of arbitrary, missing, or incorrect sequences in a target RNA molecule. The target RNA molecule may be in a cell, a tissue, an organ, or in an organism. The cell, tissue, or organ may be provided in vitro or in vivo. In some embodiments. DNA molecules provided herein can enable localization of arbitrary, missing, or incorrect sequences in RNA molecules of living cells. In some instances, the DNA molecule can comprise an localization sequence operably coupled to an antisense domain that can assist in trans-splicing of the replacement domain into RNA in order to modify (e.g., fix) the sequence. In some instances, modification or fixing of the RNA via trans-splicing can increase or decrease protein production. In some embodiments of the compositions and methods the present disclosure, a cell of the present disclosure can be a eukaryotic cell. In some embodiments, the cell can be a mammalian cell. In some embodiments, the cell can be a bovine, murine, feline, equine, porcine, canine, simian, or human cell. In some embodiments, the cell can be a non-human mammalian cell such as a non-human primate cell. In some embodiments, a cell of the present disclosure can be a somatic cell. In some embodiments, a cell of the present disclosure can be a germline cell. In some embodiments, a germline cell of the present disclosure can be not a human cell.

In some embodiments of the compositions and methods the present disclosure, a cell the present disclosure can be a stem cell some embodiments, a cell of the present disclosure can be an embryonic stem cell. In some embodiments, an embryonic stem cell of the present disclosure can be not a human cell. In some embodiments, a cell of the present disclosure can be a multipotent stem cell or a pluripotent stem cell. In some embodiments, a cell of the present disclosure can be an adult stem cell. In some embodiments, a cell of the present disclosure can be an induced pluripotent stem cell (iPSC). In some embodiments, a cell of the present disclosure can be a hematopoietic stem cell (HSC).

In some embodiments of the compositions and methods the present disclosure, an immune cell of the present disclosure can be a lymphocyte. In some embodiments, an immune cell of the present disclosure can be a T lymphocyte (also referred to herein as a T-cell). Examples of T-cells of the present disclosure can include, but are not limited to, naïve T cells, effector T cells, helper T cells, memory T cells, regulatory T cells (Tregs) and Gamma delta T cells. In some embodiments, an immune cell of the present disclosure can be a B lymphocyte. In some embodiments, an immune cell of the present disclosure can be a natural killer cell. In some embodiments, an immune cell of the present disclosure can be an antigen-presenting cell.

In some embodiments of the compositions and methods of the present disclosure, a muscle cell of the present disclosure can be a myoblast or a myocyte. In some embodiments, a muscle cell of the present disclosure can be a cardiac muscle cell, skeletal muscle cell or smooth muscle cell. In some embodiments, a muscle cell of the present disclosure can be a striated cell.

In some embodiments of the compositions and methods of the present disclosure, a somatic cell of the present disclosure can be an epithelial cell. In some embodiments, an epithelial cell of the present disclosure can form a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the present disclosure can form a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the present disclosure can contact an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the present disclosure contacts an outer surface of a blood vessel or a vein.

In some embodiments of the compositions and methods of the present disclosure, a brain cell of the present disclosure can be a neuronal cell. In some embodiments, a neuron cell of the present disclosure can be a neuron of the central nervous system. In some embodiments, a neuron cell of the present disclosure can be a neuron of the brain or the spinal cord. In some embodiments, a neuron cell of the present disclosure can be a neuron of a cranial nerve or an optic nerve. In some embodiments, a neuron cell of the present disclosure can be a neuron of the peripheral nervous system. In some embodiments, a neuron cell of the present disclosure can be a neuroglial or a glial cell. In some embodiments, a glial of the present disclosure can be a glial cell of the central nervous system including, but not limited to, oligodendrocytes, astrocytes, ependymal cells, and microglia. In some embodiments, a glial of the present disclosure can be a glial cell of the peripheral nervous system including, but not limited to, Schwann cells and satellite cells.

In some embodiments of the compositions and methods of the present disclosure, a liver cell of the present disclosure can be a hepatocytes. In some embodiments, a liver cell of the present disclosure can be a hepatic stellate cell. In some embodiments, a liver cell of the present disclosure can be Kupffer cell. In some embodiments, a liver cell of the present disclosure can be a sinusoidal endothelial cells.

In some embodiments of the compositions and methods of the present disclosure, a retinal cell of the present disclosure can be a photoreceptor. In some embodiments, a photoreceptor cell of the present disclosure is a rod. In some embodiments, a retinal cell of the present disclosure can be cone. In some embodiments, a retinal cell of the present disclosure can be a bipolar cell. In some embodiments, a retinal cell of the present disclosure can be a ganglion cell. In some embodiments, a retinal cell of the present disclosure can be a horizontal cell. In some embodiments, a retinal cell of the present disclosure can be an amacrine cell.

In some embodiments of the compositions and methods of the present disclosure, a heart cell of the present disclosure can be a cardiomyocyte. In some embodiments, a heart cell of the present disclosure can be a cardiac pacemaker cell.

In some embodiments of the compositions and methods of the present disclosure, a somatic cell of the present disclosure can be a primary cell.

In some embodiments of the compositions and methods of the present disclosure, a somatic cell of the present disclosure can be a cultured cell.

In some embodiments of the compositions and methods of the present disclosure, a somatic cell of the present disclosure can be in vivo, in vitro, ex vivo or in situ.

In some embodiments of the compositions and methods of the present disclosure, a somatic cell of the present disclosure can be autologous or allogeneic.

### Methods

The present disclosure provides a method of modifying the sequence of a target RNA molecule or a protein encoded by the target RNA molecule. The method may comprise providing a composition comprising a nucleic acid encoding a Replacement Domain. The Replacement Domain may encode or comprise an exonic sequence corresponding to a sequence of the target RNA. The method may comprise contacting the composition and the target RNA molecule under conditions suitable for binding and trans-splicing of one or more of the exonic sequence (or a portion thereof) to the target RNA molecule. The method may comprise trans-splicing with higher efficiency. As described herein, the efficiency of RNA trans-splicing may be defined as the fraction of a target RNA molecule that experiences a specific change in sequence composition that is mediated by trans-splicing. This efficiency measurement is a significant metric of therapeutic efficacy. In some embodiments, the efficiency of trans-splicing of the nucleic acid can be increased relative to the efficiency of trans-splicing of a nucleic acid that does not comprise a stabilization domain.

The present disclosure provides a method of modifying an activity of a protein encoded by an RNA molecule comprising contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 15% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 20% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 30% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 40% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 50% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 60% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 70% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 80% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an RNA molecule or a protein encoded by the RNA molecule with 90% or more efficiency, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the sequence of an untranslated region of an RNA molecule, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of increasing the expression of an RNA by insertion of WPRE or sequences with similar activity, wherein the methods can comprise contacting the composition and the RNA molecule under conditions suitable for binding and trans-splicing of one or more of the trans-splicing nucleic acids (or a portion thereof) to the RNA molecule.

The present disclosure provides a method of modifying the composition of a protein encoded by a target RNA, wherein the methods can comprise contacting the composition and a cell comprising the target RNA under conditions suitable for trans-splicing among the composition and the target RNA.

The present disclosure provides a method of modifying the composition of a target RNA with efficiency exceeding 20%, where 100% constitutes complete replacement of a chosen sequence within the target RNA, wherein the methods can comprise contacting the composition and a cell comprising the target RNA under conditions suitable for trans-splicing among the composition and the target RNA.

The present disclosure provides a method of modifying the composition of a protein encoded by a target RNA with efficiency at or about 20%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a target RNA with efficiency at or about 60%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a protein encoded by a target RNA with efficiency at or about 60%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a target RNA with efficiency at or about 70%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a protein encoded by a target RNA with efficiency at or about 70%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a target RNA with efficiency at or about 80%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a protein encoded by a target RNA with efficiency at or about 80%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a target RNA with efficiency at or about 90%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a protein encoded by a target RNA with efficiency at or about 90%, where 100% constitutes complete replacement of a chosen sequence within the Target RNA, wherein the methods can comprise contacting the composition and a cell comprising the target RNA under conditions suitable for trans-splicing among the composition and the target RNA.

The present disclosure provides a method of modifying the composition of a target RNA with high efficiency, wherein the methods can comprise contacting the composition and a cell comprising the target RNA under conditions suitable for trans-splicing among the composition and the target RNA. In some embodiments, the cell can be in vivo, in vitro, ex vivo or in situ. In some embodiments, the composition can comprise a vector comprising or encoding a trans-splicing nucleic acid molecule the present disclosure. In some embodiments, the vector is an AAV.

The present disclosure provides a method of modifying the composition of a protein encoded by a target RNA with high efficiency, wherein the methods can comprise contacting the composition and a cell comprising the target RNA under conditions suitable for trans-splicing among the composition and the target RNA. In some embodiments, the cell can be in vivo, in vitro, cx vivo or in situ. In some embodiments, the composition can comprise a vector comprising or encoding a trans-splicing nucleic acid molecule the present disclosure. In some embodiments, the vector is an AAV.

The present disclosure provides a method of treating a disease or disorder, wherein the methods can comprise administering to a subject a therapeutically effective amount of a composition the present disclosure.

The present disclosure provides a method of treating a disease or disorder, wherein the methods can comprise administering to a subject a therapeutically effective amount of a composition the present disclosure, wherein the composition can comprise a vector comprising or encoding a trans-splicing nucleic acid molecule the present disclosure, and wherein the composition can modify a level of expression of an RNA molecule the present disclosure or a protein encoded by the RNA molecule.

The present disclosure provides a method of treating a disease or disorder, wherein the methods can comprise administering to a subject a therapeutically effective amount of a composition the present disclosure, wherein the composition can comprise a vector comprising or encoding a trans-splicing nucleic acid molecule the present disclosure and wherein the composition can modify an activity of a protein encoded by an RNA molecule.

The present disclosure provides use of any of the compositions as disclosed herein for the manufacture of a medicament for the therapeutic or prophylactic treatment of any of the diseases or disorders as disclosed herein.

In some embodiments, a disease or disorder the present disclosure can include, but is not limited to, a genetic disease or disorder. In some embodiments, the genetic disease or disorder can be a single-gene disease or disorder. In some embodiments, the single-gene disease or disorder can be an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the single-gene disease or disorder is, but not limited to, common variable immune deficiency, Adenosine deaminase deficiency, X-linked severe combined immunodeficiency, Beta-thassulemia, alpha-thassulemia, myelodysplastic syndrome, Amyotrophic lateral sclerosis, Frontotemporal dementia with parkinsonism, Usher's syndrome, Krabbe disease, Niemann Pick disease, prion disease, Dravet syndrome, early-onset Parkinson's disease, spinocerebellar ataxias, genetic epilepsy disorders, Ataxia-telangiectasia, GM1 gangliosidosis, Gaucher disease, GM2 gangliosidosis, Angelman syndrome, glucose transporter deficiency type 1, Danon disease, Fabry disease. Autosomal dominant polycystic kidney disease, Pompe disease, Familial hypercholesteroletnia, Open Angle Glaucoma, Hurler syndrome or Mucopolysaccharidosis 1, Hunter syndrome or Mucopolysaccharidosis 2, Batten disease, Duchenne muscular dystrophy, Limb-girdle muscular dystrophy type 1B, Limb-girdle muscular dystrophy type 2B, Limb-girdle muscular dystrophy type 2D, Limb-girdle muscular dystrophy type 2E, Limb-girdle muscular dystrophy type 2C, Limb-girdle muscular dystrophy type 2F, Facioscapulohumeral muscular dystrophy, hemophilia B, Hemophilia A, Retinitis pigmentosa, cystic fibrosis, autosomal dominant hearing impairment, and non-syndromic hearing loss. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to. Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Nieman-Pick disease, spinal muscular atrophy, and Roberts syndrome. In some embodiments, the single-gene disease or disorder is X-linked disease or disorder including, but not limited to, muscular dystrophy, Duchenne muscular dystrophy, hemophilia, Adrenoleukodystrophy (ALD), Rett syndrome, and Hemophilia A. In some embodiments, the single-gene disease or disorder is a mitochondrial disorder including, but not limited to. Leber's hereditary optic neuropathy.

In some embodiments, a disease or disorder the present disclosure can include, but is not limited to, an immune disease or disorder. In some embodiments, the immune disease or disorder can be an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioudema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Balb disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease. Congenital heart block. Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum. Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (I-ISP), Herpes gestationis or pemphigoid gestationis (PG), Hidradenitis Suppurative (I-IS) (Acne Inversa), Hypogammaglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosis, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCU), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis. Pure red cell aplasia (PRCA), Pyoderma gangrenosum, Raynaud's phenomenon, Reactive Arthritis, Reflex sympathetic dystrophy. Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo. Vogt-Koyanagi-Harada Disease, or Wegener's granulomatosis.

In some embodiments, a disease or disorder the present disclosure can include, but is not limited to, an inflammatory disease or disorder.

In some embodiments, a disease or disorder the present disclosure can include, but is not limited to, a metabolic disease or disorder.

In some embodiments, a disease or disorder the present disclosure can include, but is not limited to, a degenerative or a progressive disease or disorder. In some embodiments, the degenerative or a progressive disease or disorder can include, but is not limited to, amyotrophic lateral sclerosis (ALS), Huntington's disease, Alzheimer's disease, and aging.

In some embodiments, a disease or disorder the present disclosure can include, but is not limited to, an infectious disease or disorder.

In some embodiments, a disease or disorder the present disclosure can include, but is not limited to, a pediatric or a developmental disease or disorder.

In some embodiments, a disease or disorder the present disclosure can include, but is not limited to, a cardiovascular disease or disorder.

In some embodiments, a disease or disorder the present disclosure can include, but is not limited to, a proliferative disease or disorder. In some embodiments, the proliferative disease or disorder is a cancer. In some embodiments, the cancer can include, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Mycloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Flaky Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma % Plasma Cell Neoplasms, Mycosis Fungoides (Lymphoma), Myelodysplastic Syndromes, Myelodysplastic Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (lead and Neck Cancer), Pheochromocytoma, Plasma Cell Neoplasm Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (plead and Neck Cancer), Sarcoma, Childhood Rhabdomyosarcoma (Soft Tissue Sarcoma), Childhood Vascular Tumors (Soft tissue Sarcoma), Ewing Sarcoma (Bone Cancer), Kaposi Sarcoma (Soft Tissue Sarcoma), Osteosarcoma (Bone Cancer), Uterine Sarcoma, Sezary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (lead and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some compositions and methods the present disclosure, a disease or disorder the present disclosure can include, but is not limited to, a proliferative disease or disorder. In some embodiments, the proliferative disease or disorder can be cancer. In some embodiments, the cancer can be caused by a virus. Cancer-causing viruses can include but are not limited to: Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), Epstein-Barr Virus (EBV). In some embodiments, the cancer can involve the presence of a gene fusion that produces a chimeric RNA with sequences derived from two genes due to a deletion or translocation of DNA. Gene fusions pairs can include but are not limited to: MAN2A1 and FER, DNAJB1 and PRKACA, BCR-ABL1, TMPRSS2 and ERG, EWSR1 and FL11, PML and RARA, EML4 and ALK, KIAA1549 and BRAF, CCDC6 and RET, SS18 and SSX1, RUNX1 and RUNX1T1, PAX3 and FOXO1, NCOA4 and RET, ETV6 and RUNX1, FUS and DDIT3, SS18 and SSX2, NPM1 and ALK, KMT2A and AFF1, TCF3 and PBX1, STIL and TAL1, COL1A1 and PDGFB, CRTC1 and MAML2, NAB2 and STATE, EWSR1 and ATF1, ETV6 and NTRK3, EWSR1 and ERG, EWSR1 and WT1, DNAJB1 and PRKACA, PAX7 and FOXO1, FUS and CREB3L2, CBFA2T3 and GLIS2, PAX8 and PPARG, KMT2A and MLLT1, EWSR1 and NR4A3, KMT2A and MLLT3, ASPSCR1 and TFE3, HMGA2 and LPP, JAZF1 and SUZ12, KIF5B and RET, FUS and ERG, SLC45A3 and ERG, NUP214 and ABL1, SET and NUP214, CD74 and ROS1, ETV6 and ABL1, TPM3 and NTRK1, PRKAR1A and RET, EWSR1 and CREB1, KMT2A and AFDN, EWSR1 and DDIT3, CLTC and ALK, ETV6 and PDGFRB, TPM3 and ALK, KMT2A and MLLT10, TMPRSS2 and ETV1, BRD4 and NUTM1, NUP98 and KDMSA, RANBP2 and ALK, CTNNB1 and PLAG1, KMT2A and EL1, TAF15 and NR4A3, FGFR3 and TACC3, PCM1 and JAK2, YWHAE and NLJTM2B, STRN and ALK, CRTC3 and MAML2, CDH11 and USP6, CDKN2D and WDFY2, CIC and DUX4, SLC34A2 and ROS1, AT1C and ALK, CD74 and NRG1, MYB and NF1B, PRCC and TFE3, KIF5B and ALK, TMPRSS2 and ETV4, KMT2A and SEPT9, EWSR1 and POUSF1, FGFR1 and PLAG1, MN1 and ETV6, TBL1XR1 and TP63, KMT2A and EPS15, SLC45A3 and ELK4, DHH and RHEBL1, HEY 1 and NCOA2, EZR and ROS1, GOPC and ROS1, HMGA2 and WIF1, KMT2A and CREBBP, SS18 and SSX4B, FAM131B and BRAE, EWSR1 and FEV, EWSR1 and PBX1, TPM4 and ALK, SND1 and BRAF, ACTB and GLI1, KMT2A and KNL1, KMT2A and SEPT6, SDC4 and ROS1, TFG and ALK, HNRNPA2B1 and ETV1, PTPRK and RSPO3, JAZF1 and PHF1, HMGA2 and RAD51B, KMT2A and MLLT11, TPR and NTRK1, AKAP9 and BRAF, FUS and CREB3L1, ETV6 and JAK2, HMGA2 and NFIB, KMT2A and AFF3, CHCHD7 and PLAG1, VTIIA and TCF7L2, LIFR and PLAG1, EWSR1 and ETV1, SRGAP3 and RAF1, KMT2A and AFF4, MEAF6 and PHF1, PAX3 and NCOA1, HAS2 and PLAG1, EWSR1 and NFATC2, HIP1 and ALK, GOLGAS and RET, BCR and JAK2, EWSR1 and ETV4, DCTN1 and ALK, MBTU1 and CXorf67, NDRG1 and ERG, CARS and ALK, SFPQ and TFE3, KMT2A and ARHGAP26, KMT2A and EP300, KMT2A and TETI, PAX5 and JAK2, PPFIBP1 and ALK, YWHAE and NUTM2A, LRIG3 and ROS1, TFG and NTRK1, TPM3 and ROS1, SLC45A3 and ETV1, ERC1 and RET, SEC 16A and NOTCH1, KTN1 and RET, SEC31A and JAK2, TCEA 1 and PLAG1, QK1 and NTRK2, RNF130 and BRAF, EIF3E and RSPO2, EWSR1 and ZNF444, LMNA and NTRK1, PPFIBP1 and ROS1, PWWP2A and ROS1, EWSR1 and YY1, FUS and ATF1, PAX3 and NCOA2, ZC3H7B and BCOR, BRD3 and NUTM1, CANT1 and ETV4, CIC and FOXO4, COL1A1 and USP6, EWSR1 and ZNF384, KMT2A and AB11, KMT2A and ACTN4, KMT2A and CEP170B, KMT2A and FOXO3, KMT2A and GAS7, KMT2A and MLLT6, KMT2A and SEPT2, KMT2A and SEPTS, MSN and ALK, VCL and ALK, EZR and ERBB4, RELCH and RET, SLC3A2 and NRG1, TRIM24 and BRAF, KLC1 and ALK, ARID1A and MAST2, GPBP1L1 and MAST2, NFIX and MAST1, NOTCH1 and GABBR2, TADA2A and MAST1, ZNF700 and MAST1, TRIM24 and RET, TRIM33 and RET, SSBP2 and JAK2, KMT2A and EEFSEC, CLCN6 and BRAF, GNAI1 and BRAF, MKRN1 and BRAF, NACC2 and NTRK2, FOFR1 and TACC1, TRIM27 and RET, HMGA2 and FHIT, HOOK3 and RET, PCM1 and RET, CEPS9 and BRAF, CLIP1 and ROS1, ERC1 and ROS1, IHLA and A and ROS1, LSM14A and BRAF, MYO5A and ROS1, SHTN1 and ROS1, TP53 and NTRK1, TPM3 and ROS1, ZCCHC8 and ROS1, FGFR3 and BAIAP2L1, KLK2 and ETV1, ACSL3 and ETV1, NUP107 and LGR5, HMGA2 and CCCNB1IP1, HMGA2 and COXWC, GATM and BRAF, HACL1 and RAF1, HERPUD1 and BRAF, ZSCAN30 and BRAF, SLC45A3 and BRAF, HMGA2 and LHFPL6, COL 1 A2 and PLAG1, ESRP1 and RAF1, IRF2BP2 and CDX1, TFG and NR4A3, CLTC and TFE3, EWSR1 and MYB, NONO and TFE3, FCHSD1 and BRAF, HMGA2 and EBF1, ACBD6 and RRP15, AGPAT5 and MCPH1, AGTRAP and BRAF, ARFIP1 and FHDC1, ATG4C and FBX038, BBS9 and PKD1L1, CENPK and KMT2A, CNBP and USP6, DDX5 and ETV4, EIF3K and CYP39A 1, EPC 1 and PHF1, ERO1A and FERMT2, ETV6 and ITPR2, EWSR1 and NFATC1, EWSR1 and PATZ1, EWSR1 and SMARCA5, EWSR1 and SP3, FBXL18 and RNF216, FGFR1 and ZNF703, FN1 and ALK, FUS and FEV, GMDS and PDE8B, HMGA2 and ALDH2, IL6R and ATP8B2, INTS4 and GAB2, JPT1 and USH1G, KLK2 and ETV4, KMT2A and ABI2, KMT2A and ARHGEF12, KMT2A and BTBD18, KMT2A and CASPSAP2, KMT2A and CBL, KMT2A and CIP2A, KMT2A and CT45A2, KMT2A and DAB21P, KMT2A and FOXO4, KMT2A and FRYL, KMT2A and GMPS, KMT2A and GPHN, KMT2A and LASP1, KMT2A and LPP, KMT2A and MAPRE1, KMT2A and MYO1F, KMT2A and NCK1PSD, KMT2A and NRIP3, KMT2A and PDS5A, KMT2A and PICALM, KMT2A and PRRC1, KMT2A and SARNP, KMT2A and SH3GL1, KMT2A and SORBS2, KMT12A and TOP3A, KMT2A and ZFYVE19, MBOAT2 and PRKCE, MIA2 and GEMIN2, NF1 and ASIC2, NFIA and EHF, NTN1 and ACLY, OMD and USP6, PLA2R1 and RBMS1, PLXND1 and TMCC1, RAF1 and DAZL, RBM14 and PALS1, RGS22 and SYCP1, SEC31A and ALK, SEPT8 and AFF4, SLC22A1 and CUTA, S1,C26A6 and PRKAR2A, SLC45A3 and ETV5, SQSTM1 and ALK, SS18L1 and SSX1, SSH2 and SUZ12, SUSD1 and PTBP3, TCF12 and NR4A3, TECTA and TBCEL, THRAP3 and USP6, TMPRSS2 and ETV5, TPR and ALK, UBE2L3 and KRAS, WDCP and ALK, SS18 and USP6.

In some methods the present disclosure, a subject the present disclosure can be diagnosed with the disease or disorder. In some embodiments, the subject the present disclosure can present at least one sign or symptom of the disease or disorder. In some embodiments, the subject can have a biomarker predictive of a risk of developing the disease or disorder. In some embodiments, the biomarker can be a genetic mutation.

In some methods the present disclosure, a subject the present disclosure can be female. In some embodiments of the methods the present disclosure, a subject the present disclosure can be male. In some embodiments, a subject the present disclosure can have two XX or XY chromosomes. In some embodiments, a subject the present disclosure can have two XX or XY chromosomes and a third chromosome, either an X or a Y.

In some methods the present disclosure, a subject the present disclosure can be a neonate, an infant, a child, an adult, a senior adult, or an elderly adult. In some embodiments of the methods the present disclosure, a subject the present disclosure can be at least 1, 2, 3.4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods the present disclosure, a subject the present disclosure can be at least 1, 2, 3, 4, 5, 6, 7, 8, 9, 10, 11 or 12 months old. In some embodiments of the methods the present disclosure, a subject the present disclosure can be at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some methods the present disclosure, a subject the present disclosure can be a mammal. In some embodiments, a subject the present disclosure can be a non-human mammal.

In some methods the present disclosure, a subject the present disclosure can be a human.

In some methods the present disclosure, a therapeutically effective amount can comprise a single dose of a composition the present disclosure. In some embodiments, a therapeutically effective amount can comprise a therapeutically effective amount can comprise at least one dose of a composition the present disclosure. In some embodiments, a therapeutically effective amount can comprise a therapeutically effective amount can comprise one or more dose(s) of a composition the present disclosure.

In some methods the present disclosure, a therapeutically effective amount can eliminate a sign or symptom of the disease or disorder. In some embodiments, a therapeutically effective amount can reduce a severity of a sign or symptom of the disease or disorder.

In some embodiments of the methods the present disclosure, a therapeutically effective amount can eliminate the disease or disorder.

In some methods the present disclosure, a therapeutically effective amount can prevent an onset of a disease or disorder. In some embodiments, a therapeutically effective amount can delay the onset of a disease or disorder. In some embodiments, a therapeutically effective amount can reduce the severity of a sign or symptom of the disease or disorder. In some embodiments, a therapeutically effective amount can improve a prognosis for the subject.

In some methods the present disclosure, a composition the present disclosure can be administered to the subject systemically. In some embodiments, the composition the present disclosure can be administered to the subject by an intravenous route. In some embodiments, the composition the present disclosure can be administered to the subject by an injection or an infusion.

In some methods the present disclosure, a composition the present disclosure can be administered to the subject locally. In some embodiments, the composition the present disclosure can be administered to the subject by an intraosseous, intraocular, intracerebrospinal or intraspinal route. In some embodiments, the composition the present disclosure can be administered directly to the cerebral spinal fluid of the central nervous system. In some embodiments, the composition the present disclosure can be administered directly to a tissue or fluid of the eye and does not have bioavailability outside of ocular structures. In some embodiments, the composition the present disclosure can be administered to the subject by an injection or an infusion.

### Pharmaceutical Compositions

In some embodiments, the compositions comprising the trans-splicing nucleic acids disclosed herein can be formulated as pharmaceutical compositions. Briefly, pharmaceutical compositions for use as disclosed herein may comprise a fusion protein(s) or a polynucleotide encoding the fusion protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol: proteins: polypeptides or amino acids such as glycine; antioxidants; chelating agents such as EDTA or glutathione: adjuvants (e.g., aluminum hydroxide); and preservatives. Compositions of the present disclosure may be formulated for oral, intravenous, topical, enteral, intraocular, and/or parenteral administration. In certain embodiments, the compositions of the present disclosure are formulated for intravenous administration.

### EXAMPLES

The following examples are included for illustrative purposes only and are not intended to limit the scope of the invention.

### Example 1: Trans-Splicing Mechanism

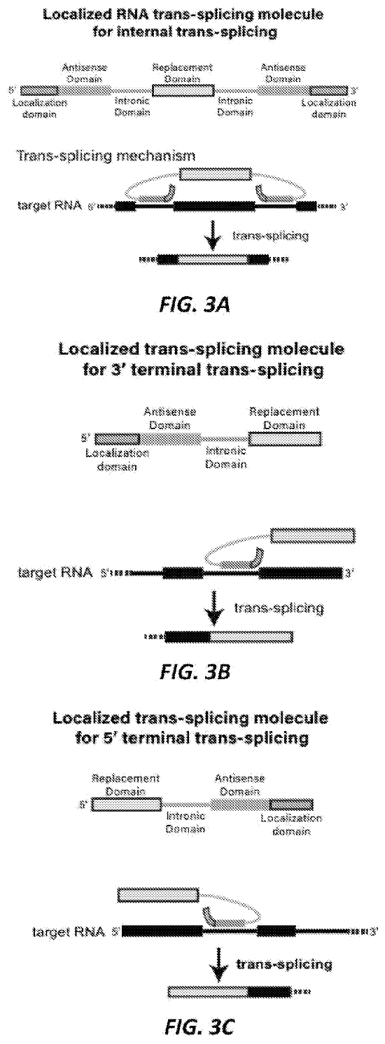

Compositions as used herein may comprise a double trans-splicing molecule comprising two antisense domains, one replacement domain, two intronic domains, and at least one Localization Domain at the 5′ and/or 3′ end of the trans-splicing molecule (FIG. 3A). This design can promote replacement of an internal sequence within the target RNA while maintaining the adjacent 5′ and 3′ sequences around the replaced sequence. In some cases, terminal trans-splicing molecules may comprise one antisense domain, one replacement domain, one intronic domain, and at least one Localization Domain at the 5′ and/or 3′ end of the trans-splicing molecule. The design of a 3′ terminal trans-splicing nucleic acid can replace the 3′ terminal end of a target RNA while maintaining the 5′ end (FIG. 3B), while the design of a 5′ terminal trans-splicing molecule can replace the 5′ terminal end of a target RNA while maintaining the 3′ end (FIG. 3C).

### Example 2: Identification of Localization Sequences for Trans-Splicing Molecules

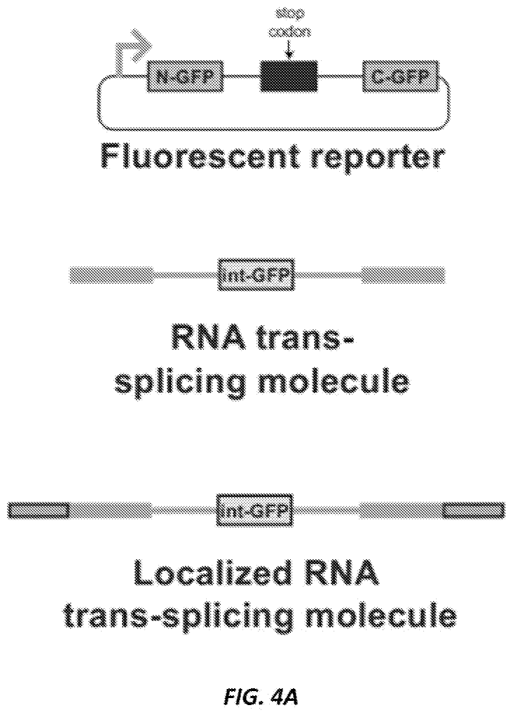

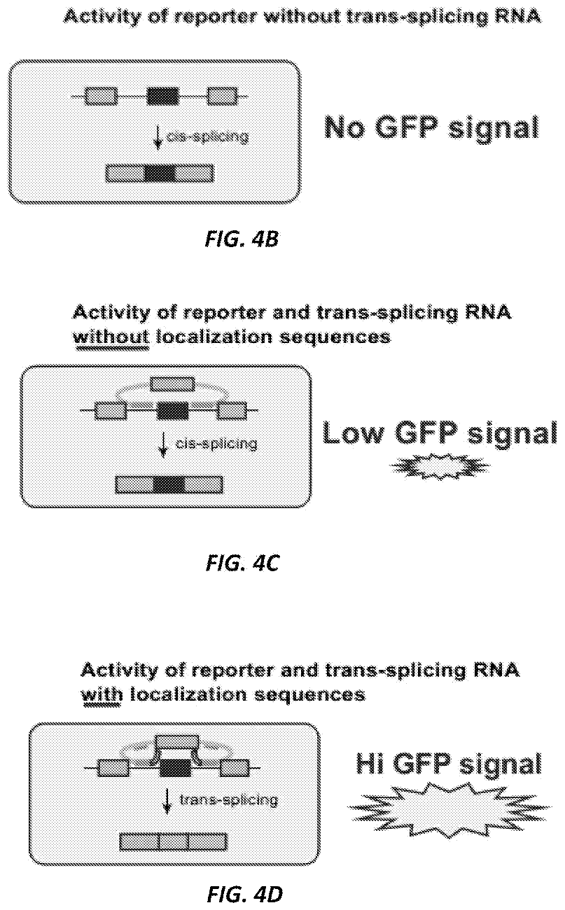

Trans-splicing molecules as disclosed herein may target a split GFP reporter RNA that fluoresces only after successful activity of the RNA trans-spicing molecule (FIGS. 4-6). This assay is qualitative, not fully quantitative, but is useful because it is what end-users in cell biology often use when attempting to answer scientific questions about the presence, absence, or general magnitude of a transcript. GEP trans-splicing reporters has, accordingly, been widely used in the study of RNA trans-splicing technologies. A GFP reporter similar to a published system (Koller et al., 2011, which is incorporated herein by reference in its entirety) was used to compare the relative influence of different sequences on the efficiency of the tram-splicing reaction.

FIGS. 4-6 comprise a schematic of the plasmids used in the trans-splicing activity assays.

An experiment was designed to ascertain the importance of localization sequences in the context of internal trans-splicing via production of GFP protein (FIG. 4). For example, the design of a split GFP reporter that carries N- and C-terminal portions of GEP (“N-GFP” and “C-GFP”) but lacks an internal GFP sequence required for fluorescence can be used to understand the effects of localization sequences. In the reporter, this internal sequence is replaced by a short exon with a stop codon that is flanked by introns. The internal sequence (“int-GFP”) is the replacement sequence within an RNA trans-splicing molecule that is flanked by two intronic sequences, two antisense sequences, and one or more localization sequences. FIG. 4B illustrates the activity of the reporter alone so that cis-splicing produces a GFP sequence interrupted by a stop codon therefore producing no GFP signal.

FIG. 4C illustrates the activity of the reporter in the presence of the trans-splicing molecule without inclusion of localization sequences in the trans-splicing molecule so that similarly cis-splicing occurs primarily and GFP signal is not efficiently generated. This is because localization sequences that promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the lack of nuclear localization sequences may result in less accumulation of trans-slicing RNA to the site of transcription, thereby resulting in lower trans-splicing efficiency.

FIG. 4D illustrates the activity of the reporter in the presence of the trans-splicing molecule with inclusion of localization sequences so that trans-splicing occurs primarily and GFP signal is efficiently produced. Localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the inclusion of nuclear localization sequences may result in greater accumulation of trans-slicing RNA to the site of transcription, thereby resulting in greater trans-splicing efficiency.

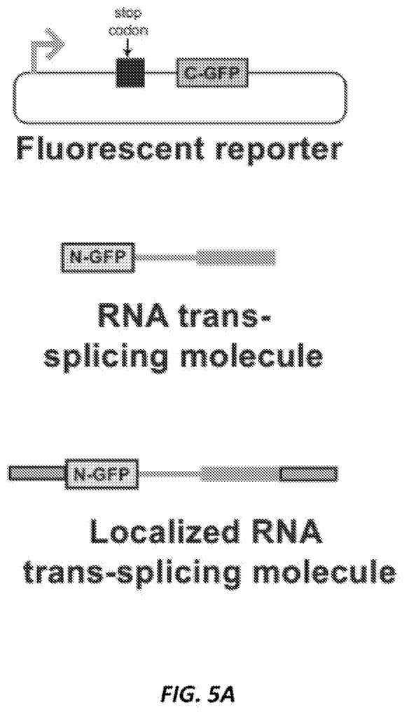

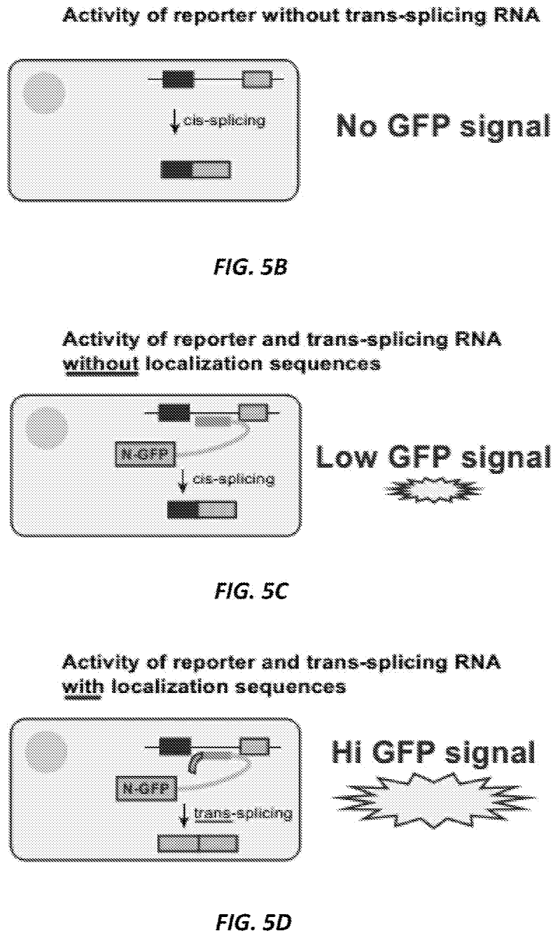

FIGURES illustrates an experiment designed to reveal the importance of localization sequences in the context of 5′ terminal trans-splicing. FIG. 5A illustrates the design of a split GFP reporter that carries a C-terminal portion of GFP (“C-GFP”) but lacks an N-terminal GFP sequence required for fluorescence. In the reporter, this N-terminal GFP sequence is replaced by a short exon with a stop codon that is flanked by introns. The N-terminal sequence (“N-GFP”) is the replacement sequence within an RNA trans-splicing molecule that is flanked by one intronic sequence, one antisense sequence, and one or more and one or more localization sequences. FIG. 5B illustrates the activity of the reporter alone so that cis-splicing produces a GFP sequence interrupted by a stop codon therefore producing no GFP signal.

FIG. 5C illustrates the activity of the reporter in the presence of the trans-splicing molecule without inclusion of localization sequences in the trans-splicing molecule so that similarly cis-splicing occurs primarily and GFP signal is not efficiently produced. The results of FIGS. 5B and 5C occur, in part, because localization sequences promote the accumulation of the trans-splicing nucleic acid to the site of transcription. As RNA splicing occurs in close coordination with transcription, this accumulation of the RNA trans-splicing molecule at the site, of transcription increases RNA trans-splicing efficiency. Thus, the lack of nuclear localization sequences may result in less accumulation of trans-slicing RNA to the site of transcription, thereby resulting in lower trans-splicing efficiency.

FIG. 5D illustrates the activity of the reporter in the presence of the trans-splicing molecule with inclusion of localization sequence so that trans-splicing occurs primarily and GFP signal is efficiently produced. Localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Deus, the inclusion of nuclear localization sequences may result in greater accumulation of trans-slicing RNA to the site of transcription, thereby resulting in greater trans-splicing efficiency.

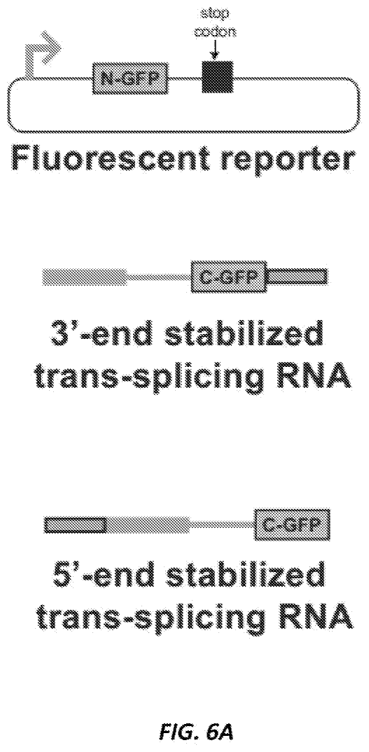

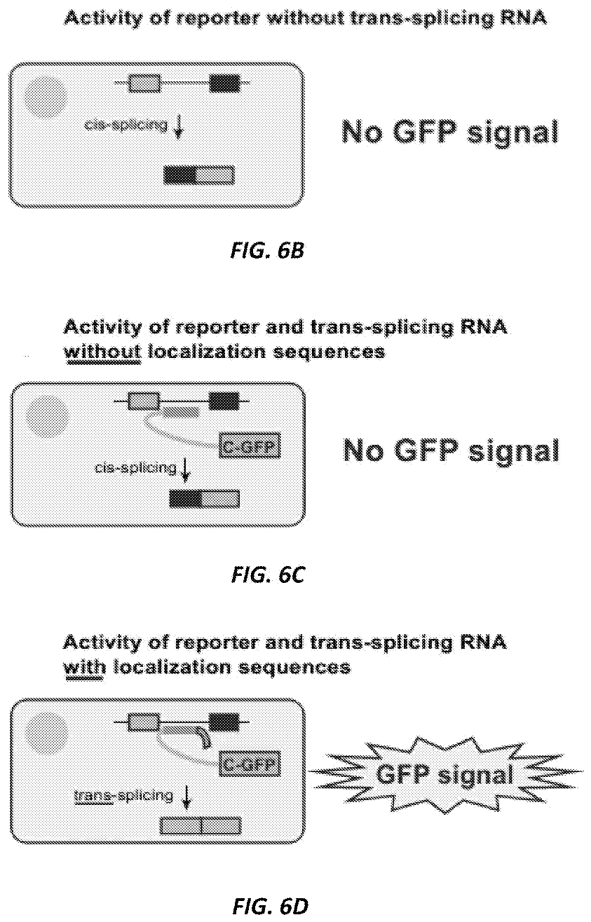

FIG. 6 illustrates an experiment designed to reveal the importance localization sequences in the context of 3′ terminal trans-splicing. FIG. 6A illustrates the design of a split GFP reporter that carries a N-terminal portion of GFP (“N-GFP”) but lacks an C-terminal GFP sequence required for fluorescence. In the reporter, this C-terminal GFP sequence is replaced by a short exon with a stop codon that is flanked by introns. The C-terminal sequence (“C-Glop”) is the replacement sequence within an RNA trans-splicing molecule that is flanked by one intronic sequence, one antisense sequence, and one or more and one or more localization sequences.

FIG. 6B illustrates the activity of the reporter alone so that cis-splicing produces a GFP sequence interrupted by a stop codon therefore producing no GFP signal. FIG. 6C illustrates the activity of the reporter in the presence of the trans-splicing molecule without inclusion localization sequences in the trans-splicing molecule so that similarly cis-splicing occurs primarily and GFP signal is not efficiently produced. The results of FIGS. 6B and 6C occur, in part, because localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the lack of nuclear localization sequences may result in less accumulation of trans-slicing RNA to the site of transcription, thereby resulting in lower trans-splicing efficiency.

FIG. 6D illustrates the activity of the reporter in the presence of the trans-splicing molecule with inclusion of localization sequences so that trans-splicing occurs primarily and GFP signal is produced. Localization sequences promote the accumulation of the trans-splicing nucleic acid to the site of transcription. As RNA splicing occurs in close coordination with transcription, this accumulation of the RNA trans-splicing molecule at the site of transcription increases RNA trans-splicing efficiency. Thus, the inclusion of nuclear localization sequences may result in greater accumulation of trans-slicing RNA to the site of transcription, thereby resulting in greater trans-splicing efficiency.

Experiments were conducted with either a transiently-transfected reporter and trans-splicing molecule or systems packaged in lentivirus. Trans-splicing molecules comprising sequences that are known to promote nuclear or subnuclear localization of RNAs were transiently-transfected in II K293T cells and RNA harvested in order to assess whether the presence of putative localization sequences resulted in increased the efficiency of the trans-splicing molecules. RNA was subjected to reverse transcription and quantitative PCR using primers that amplify the trans-splicing molecule and a housekeeping gene. Indeed, localization sequences increased the levels of the trans-splicing molecule.

### Example 3: Localization of Sequences

To investigate the activity of localization sequences on trans-splicing molecule efficiency, experiments were conducted to measure the efficiency editing of two endogenous genes: Scn1a and Dmd. Mutations in these genes cause Dravet syndrome and Duchenne muscular dystrophy, respectively. The transfected cell lines that express these genes with trans-splicing molecules that comprise localization sequences and which target each of these genes in order to assess the influence of localization sequences on trans-splicing efficiency were used. Specifically. RNA from the transfected or infected Neuro-2A cells with trans-splicing molecules targeting Scn1a carrying localization sequences were extracted from these cells 48 hours later and subjected the RNA reverse transcription and quantitative PCR using primers that amplify the trans-splicing molecule and a housekeeping gene. Additionally. RNAs from the transfected or infected C2C12 cells with trans-splicing molecules targeting Dmd carrying localization sequences were extracted. The RNAs were subsequent to reverse transcription, followed by PCR measurement. In both instances, cells with localization sequence exhibited increased levels of the trans-splicing molecule.

In order to further investigate the activity of localization sequences on trans-splicing molecule efficiency, experiments were conducted in a mouse models of Dravet syndrome. Specifically, mice carrying mutations in exon 1 of Scn1a that display frequent and fatal seizures (129S-Scn1atm1Kea/Mmjax) were treated with adeno-associated virus (AAV) encoding trans-splicing molecules that carry localization sequences. Specifically, AAV was administered via direct brain injection or via intracerebroventricular injection within the first month of life. Next, seizure frequency and survival of mice was measured. Mice treated with AAV encoding the trans-splicing molecule carrying localization sequences displayed reduced seizure frequency and greater survival than untreated mice or mice treated with a control AAV that did not have a trans-splicing molecule.

In order to further investigate the activity of localization sequences on trans-splicing molecule efficiency, experiments were conducted in a mouse models of Duchenne muscular dystrophy syndrome. Specifically, mice carrying mutations in exon 10 of Dmd that experience muscle degeneration and eventual death (B6Ros.Cg-Dmdmdx-5Cv/J) were treated with adeno-associated virus (AAV) encoding trans-splicing molecules that carry localization sequences. Specifically, AAV was administered via intramuscular injection or via systemic injection within the first month of life. Next, various measurements of muscle strength such as rotarod assay and survival of mice were measured. Mice treated with AAV encoding the trans-splicing molecule carrying localization sequences displayed increased strength and greater survival than untreated mice or mice treated with a control AAV that did not have a trans-splicing molecule.

### Example 4: Delivery Replacement Gene

Described herein is systems, methods, and compositions that can be used to deliver a replacement gene by trans-splicing molecule described herein.

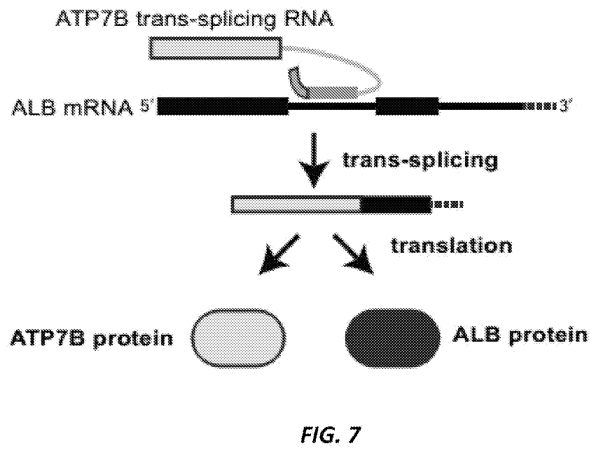

FIG. 7 schematically illustrates a concept whereby trans-splicing can be used in the context of a gene therapy. The replacement gene is ATP7B, a gene that is primarily expressed in the liver and mutated in Wilson's disease. The ATP7B trans-splicing molecules will comprise of (1) a localization sequence, (2) an antisense domain, (3) an intronic domain, and (4) a replacement domain (e.g., ATP7B). Upon successful trans-splicing the ATP7B coding sequence into a liver-specific and highly-expressed gene (e.g., ALB) the ATP7B gene expression can be generated in the liver only.

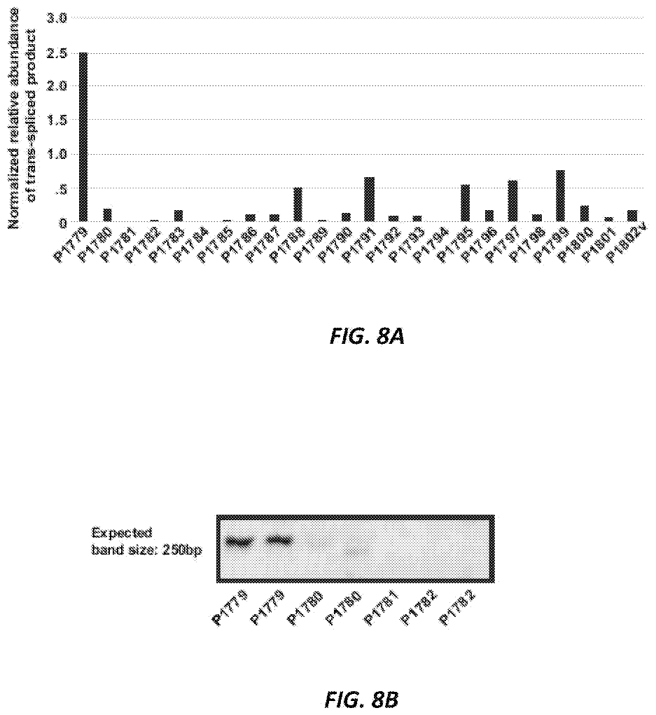

FIG. 8 describes the influence of various long-noncoding RNA (lncRNA) sequences on the activity of a trans-splicing nucleic acid that targets the human ALB gene. Each bar represents a different trans-splicing molecule that is identical except for the addition of a human lncRNA sequence. The level of trans-spliced RNA product was assessed using RT-PCR with primers that target the trans-spliced product exclusively. The sequence of trans-splicing molecules P1779-P1802 are listed as SEQ ID NOs: 21-43.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-2. (canceled)

3. A composition comprising a nucleic acid, comprising a sequence encoding: (a) a replacement domain that encodes a therapeutic sequence; (b) an intronic domain configured to promote ribonucleic acid (RNA) splicing of the replacement domain; (c) an antisense domain configured to promote binding to a target RNA molecule; and (d) a localization domain configured to promote accumulation of the nucleic acid in the cellular nucleus as compared to a nucleic acid without the localization domain.

4-104. (canceled)
